SEQUENCE LISTING (15 sequences)

SEQ ID NO: 1 - protein, 143 aa, homo sapien
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 2 - protein, 144 aa, homo sapien
CGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 3 - protein, 250 aa, homo sapien
MADNFSLHDALSGSGNPNPQGWPGAWGNQPAGAGGYPGASYPGAYPGQAPPGAYPGQAPPGAYHGAPGAYPGAPAPGVYPGPPSGPGAYPSSGQPSAPGAYPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 4 - protein, 143 aa, homo sapien
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPEHFKVAVNDAHLLQYNHRVKKLNEISKLGISGDIDLTSASYTMI

SEQ ID NO: 5 - protein, 143 aa, homo sapien
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPEHFKVAVNDAHLLQYNHRAKKLNEISKLGISGDIDLTSASYTMI

SEQ ID NO: 6 - DNA, 27 bp, artificial sequence
  primer
gacgacgacaagggcgcccctgctggg

SEQ ID NO: 7 - DNA, 30 bp, artificial sequence
  primer
gaggagaagcccggtttatatcatggtata

SEQ ID NO: 8 - DNA, 30 bp, artificial sequence
  primer
gacgacgacaagtgcggcgcccctgctggg

SEQ ID NO: 9 - DNA, 30 bp, artificial sequence; identical to SEQ ID NO: 7
  primer

SEQ ID NO: 10 - DNA, 36 bp, artificial sequence
  primer
gacgacgacaagtgcggcgcccctgctgggccactg

SEQ ID NO: 11 - DNA, 27 bp, artificial sequence
  primer
actgttgaacctgaacacttcaaggtt

SEQ ID NO: 12 - DNA, 27 bp, artificial sequence
  primer
aaccttgaagtgttcaggttcaacact

SEQ ID NO: 13 - DNA, 27 bp, artificial sequence
  primer
tacaatcatcgggctaaaaaactcaat

SEQ ID NO: 14 - DNA, 27 bp, artificial sequence
  primer
actgttgaacctgaacacttcaaggtt

SEQ ID NO: 15 - DNA, 818 bp, homo sapien
agcagccgtccggagccagccaacgagcggaaaatggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccatggagcacctggagcttatcccggagcacctgcacctggagtctacccagggccacccagcggccctggggcctacccatcttctggacagccaagtgcccccggagcctaccctgccactggcccctatggcgcccctgctgggccactgattgtgccttataacctgcctttgcctgggggagtggtgcctcgcatgctcataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgatataatctgaaaggggcagattaaaaaaaaaaacgga

(19) United States
(12) Patent Application Publication
(10) Pub. No.: US 2005/0032673 A1
(43) Pub. Date: Feb. 10, 2005

(54) Sustained release N-terminally truncated galectin-3 and antibodies to galectin-3 carbohydrate ligands for use in treating disease

(76) Inventors: Constance M. John, San Francisco, CA (US); Gary A. Jarvis, San Francisco, CA (US); Hakon Leffler, Lund (SE)

Correspondence Address: Amy E. Rinaldo;KOHN & ASSOCIATES, PLLC, Suite 410, 30500 Northwestern Highway, Farmington Hills, MI, 48334, US

(21) Appl. No.: 10/726,198

(22) Filed: Dec. 2, 2003

Related U.S. Application Data
(63) Continuation-in-part of application No. PCT/US02/18478, filed on Jun. 10, 2002.
(60) Provisional application No. 60/430,253, filed on Dec. 2, 2002.

(51) Int. Cl. A61K 38/17

(52) U.S. Cl. 514/8

(57) ABSTRACT

There is provided a composition for anti-cancer or anti-inflammatory treatment having an effective amount of N-terminally truncated galectin-3 or its homologues, antibodies to galectin-3 carbohydrate binding sites, or a nucleic acid sequences encoding N-terminally truncated galectin-3 or its homologues, in a pharmaceutically acceptable carrier. Also provided by the present invention is a method of treating disease by administering to a patient in need of such treatment an effective amount of N-terminally truncated galectin-3, its homologues, antibody to galectin-3 carbohydrate binding sites, or a nucleic acid sequence encoding N-terminally truncated galectin-3 in a pharmaceutically acceptable carrier. Further, there is provided a disease treatment having an effective amount of N-terminally truncated galectin-3 or its homologues, antibodies to galectin-3 carbohydrate binding sites, or a nucleic acid sequences encoding N-terminally truncated galectin-3 or its homologues, in a pharmaceutically acceptable carrier.

### CROSS-REFERENCE TO RELATED APPLICATIONS

This application claims the benefit of priority under 35 U.S.C. Section 119(e) of United States Provisional Patent Application No. 60/430,253, filed Dec. 2, 2002, and Continuation-In-Part Application No. PCT/US02/18478, filed Jun. 10, 2002, both of which are incorporated herein by reference.

### BACKGROUND OF THE INVENTION

1. Technical Field

The present invention relates to methods and compositions for treating cancer and conditions or diseases involving inflammation, undesirable immunity, and infection. More specifically, the present invention relates to a composition containing N-terminally truncated galectin-3 homologues of N-terminally truncated galectin-3, an effective amount of a nucleic acid encoding N-terminally truncated galectin-3 or its homologues, and antibodies to galectin-3 carbohydrate binding sites for treating disease.

2. Background Art

Lectins are, by definition, proteins with at least one carbohydrate-binding domain. By immobilizing monosaccharides, oligosaccharides, or glycoproteins in affinity columns, lectins have been isolated from tumor tissue extracts. Generally, a tissue extract in acetone or the like is utilized to isolate the protein component from the lipid component. The acetone is then evaporated, whereupon the residue is solublized in a buffered aqueous solution. This solution is then passed through an affinity column containing the immobilized carbohydrates or glycoproteins. A number of lectins, which selectively bind to galactosides, have been isolated in this manner.

Galectin-3 is one member of the family of lectins termed galectins, formerly known as S-type or S-Lac lectins. Galectins are classified as such due to their structural similarity and characteristic affinity for b-galactoside sugars (1,2). The highest levels of galectin-3 are found in activated macrophages, basophils, mast cells, some epithelial cells, and sensory neurons. An early observation was that many tumor cells express galectins on their surface and that their expression could be involved in adhesion and invasion processes. Experimental evidence also suggested that these galectins could be cross-linked by an exogenous glycoprotein resulting in the aggregation of tumor cells. Based on these results, Raz and Lotan proposed that galectin-1 and galectin-3 could promote tumor metastasis (3). Since that time, the evidence for the role of galectin-3 in tumor adhesion, invasion and metastasis has mounted.

Galectin-3 is composed of three distinct structural motifs: a short amino terminal region of 12 amino acids, a sequence rich in Gly-X-Tyr tandem repeats characteristic of the collagen supergene family, and a carboxy-terminal half containing the globular carbohydrate recognition domain (CRD) (2,4,5). There is close homology between the galectin-3 proteins of different species, but the number of N-terminal tandem repeats differs, and therefore, the sizes of the proteins vary (6). The human protein is composed of 250 amino acid residues with a Mᵣ of ˜31,000 based on electrophoretic mobility (7), and a CRD that extends from residue 117 to 250 (8). The X-ray crystal structure of the galectin-3C protein complexed with lactose and N-acetyllactosamine has been published. The publication revealed that the CRD includes hydrogen binding-of specific amino acid residues, the first being Arg-144 and the last being Glu-184 (9). The first residue with electron density was Leu-114, consequently, the first 6 residues were thought to be disordered (8).

A study of the minimal folding domain of galectin-3, required for lactose binding, was accomplished using a peptide library formed by cloning galectin-3 cDNA into a phage vector. The library was screened by affinity selection using lactose immobilized on agarose beads. DNA sequence analysis of the clones isolated by their affinity for immobilized lactose defined the minimal domain as about 136 amino acid residues, beginning with Ile-115 and extending through Ile-250 (9). Further deletions of 10 or 15 amino acids from either end abolished both lactose binding and stable expression in bacteria. It is possible that lactose binding can be retained with deletions of fewer than 10 amino acid residues especially since the Ile-115 residue was not involved in direct interaction with lactose in the crystal structure. There was electron density indicative of such interaction around the carboxyl-terminal Ile-250 (8).

Although all galectins bind lactose with similar affinity, each galectin is more specific and has higher affinity for certain more complex saccharides (10,11). Galectins, in general, are unusual among extracellular proteins in that they are initially mainly cytosolic but can be secreted by non-classical pathways, translocated to the cell nucleus, and endocytosed and trancytosed by cells. Galectins are thought to interact with various cell-surface and extracellular glycoproteins and glycolipids, thereby playing a role in cell adhesion, migration, and signaling. The relationship between the intra- and extracellular functions of galectins can be of great biological importance. A number of reviews of the biology of the galectins have been published (12-19).

Studies of mutants of hamster galectin-3 with various deletions in the N-terminal domain have shown that, even if lacking the first 103 amino acid residues, the protein is localized in the nucleus. Deletion of the first 110 amino acid residues, however, prevented nuclear localization, although the exact sequence of amino acid residues 104-110, APTGALT, was not obligatory and substitution of other unrelated sequences permitted nuclear sequestration (20). The amino acid residues 104-110 of the hamster galectin-3 protein, according to the consensus sequence, correspond to the amino acid residues 109-115 of the homologous human galectin-3 protein (20). Loss of nuclear localization of galectin-3 was observed in senescent human fibroblasts (21), and binding to synexin was found to be associated with its nuclear transport (22).

Galectin-3 shares the ability to be secreted despite the absence of a signal peptide with a number of other proteins that have unconventional intercellular transfer. These proteins are internalized by cells and are able to directly access the cytoplasm and the nucleus by a process that does not involve classical endocytosis (23). This is in contrast with the modulation of intercellular events by second messengers that bind to extracellular receptors and initiate a cascade of intracellular events that often involve transcriptional regulation. Although the mechanisms for the ability of some proteins to cross biological membranes in the absence of a signal sequence are poorly understood, a number of common features have been identified. Many of the proteins can directly access the nucleus. The mechanisms for secretion of the proteins often vary from the mechanisms for entry. Additionally, apolipoproteins and cholesterol can play a role in the mechanisms for entry (23).

Galectin-3 is isolated as a monomer but undergoes multimerization upon binding to surfaces that contain glycoconjugate ligands. The N-terminal domain of the protein is required for this property (24,25). The N-terminal domain of the protein is also required for galectin-3 to have affinity for multivalent carbohydrate ligands (24,26) and to transmit intracellular signals (27,28). Galectin-3 promotes binding of cells to laminin and fibronectin, but the N-terminally truncated protein does not promote such binding (29). Thus, the N-terminal domain appears to be necessary for the self-association of galectin-3 that is required for some of its biological functions. In order to establish this principle, Galectin-3 null cells were transfected to express recombinant galectin-3. The recombinant galectin-3 induced tumors within four weeks when injected into mice. When the same galectin-3 null cells were transfected to express a mutant galectin-3 that was lacking the 11 amino terminal amino acids, no tumors developed within four weeks (27).

A number of laboratories have studied the biology of galectin-3 that apparently is significant in cell growth, differentiation, adhesion, RNA processing, apoptosis, and malignancy transformation (30). Laminin is the major non-collagenous polypeptide of basement membranes, and galectin-3 binds preferentially to mouse tumor laminin compared to human placental laminin (31). Galectin-3 has been shown to increase the binding of breast cancer cells to other extracellular matrix proteins (32,33). In addition to increasing the binding of tumor cells to basement membranes, the interaction of cell surface galectin-3 with complementary serum glycoproteins appears to promote aggregation of tumor cells in circulation, thereby playing another important role in the pathogenesis of metastasis (34). Galectin-3 and 3-C have been shown to have higher affinity for polylactosaminylated ligands than other members of the galectin family (35).

Altered glycosylation of glycoproteins and glycolipids is one of the molecular changes that accompany malignant transformation (36). Tumor growth and metastasis was suppressed in mice deficient in α1,6-N-acetylglcosaminyltrasferase V (MGAT5), an enzyme that catalyzes the addition of 1,6GlcNAc to form tri- and tetra antenna-like oligosaccharides that are often modified further to form polylactosamines (37,38).

Expression of recombinant galectin-3 in weakly metastatic fibrosarcoma cells resulted in an increased incidence of experimental lung metastases in syngeneic and nude mice (39). In human umbilical vein endothelial cells (HUVEC) galectin-3 induces angiogenesis (40). Increased expression of galectin-3 in human colon cancer cells resulted in increased metastases, and reduction of galectin-3 expression from antisense DNA was associated with decreased liver colonization and spontaneous metastasis in athymic nude mice (41). Exogenous galectin-3 has been shown to increase invasiveness of human breast cancer cells, and to be a chemotactic factor for human umbilical vein endothelial cells (41). Introduction of human galectin-3 cDNA into the human breast cancer cells BT-549, which are galectin-3 null and non-tumorigenic in nude mice, resulted in the establishment of four galectin-3 expressing clones, three of which acquired tumorigenicity when injected into nude mice (42). Nonetheless, the role of galectin-3 in cancer is complicated, and a number of different laboratories have found that decreased expression of galectin-3 is associated with increased tumorigenicity and metastasis (43-46). Overall, the body of work regarding the biochemistry and function of galectin-3 provides a strong rationale for continued exploration of the therapeutic use of galectin-3 in cancer.

Galectin-3 is not a member of the Bcl-2 family of proteins, but at residues 180-183 it contains the four amino acid motif (NWGR) conserved in the BH1 domain of the Bcl-2 family, and it has 48% sequence similarity with Bcl-2 (47). Galectin-3 has an anti-apoptotic activity that is abrogated by substitution of the Gly182 residue with Ala in the NWGR motif (48,49). In T-cells, galectin-3 interacts with Bcl-2 in a lactose inhibitable manner and confers resistance to apoptosis induced by anti-Fas antibody and staurosporine (47). Galectin-3 has been found to improve cellular adhesion and prevent apoptosis induced by loss of cell anchorage (anoikis) (49-51). Contact with the extracellular matrix is required for suppression of apoptosis of epithelial cells from a number of tissues.

By providing a mechanism for adherence of tumor cells to one another and to the extracellular matrix (29) and the subsequent suppression of apoptotis, galectin-3 on the surface of tumor cells appears to contribute to tumor invasion and metastasis. This premise is supported by the inhibition of spontaneous metastasis in a rat prostate cancer model by oral administration of modified citrus pectin, a complex polysaccharide rich in galactosyl residues. Citrus pectin (pH modified), a plant fiber component, can directly bind galectin-3, and can interfere with carbohydrate-mediated cell-cell and cell-matrix interactions (52).

Galectin-3 is expressed by some cells of the immune system including activated T lymphocytes (53), eosinophils (54), macrophages (55-58), and neutrophils (54). Galectin-3 enhances the adaptive immune response by facilitation of antigen presentation and by increasing the proliferation of mature lymphocytes (reviewed in [Ochieng, 1995 #977]). The expression of anti-sense but not sense phosphorothioated oligonucleotides for galectin-3 significantly blocked proliferation of T-cells (53). In T-cells galectin-3 interacts with Bcl-2 in a lactose inhibitable manner and confers resistance to apoptosis induced by anti-Fas antibody and staurosporine (47). Further, lactose and rabbit anti-galectin-3 serum inhibited the production of IgE by B-cells that was stimulated in vitro by polymorphonuclear leukocytes isolated from patients with IgE-associated atopic eczema/dermatitis syndrome (AEDS) (59).

Galectin-3 modulates thymocyte interactions with the thymic microenvironment. Expression of galectin-3 in the thymus of young adult mice was mainly in the medulla and to a lesser extent in the cortex. Distinct microenvironmental elements produced, secreted, and accumulated galectin-3 on the cell surface. Galectin-3-enriched medium inhibited in vitro thymocyte (T-cell) interactions with thymic microenvironmental cells, accelerated the release of thymocytes from thymic nurse cells, and inhibited the reconstitution of lymphoepithelial complexes (60).

The MGAT5 deficient mice also showed kidney autoimmune disease, enhanced delayed-type hypersensitivity, increased susceptibility to experimental autoimmune encephalomyelitis, and increased susceptibility to kidney autoimmune disease (61). Pre-treatment of wild-type T-cells with lactose to compete for galectin binding produced a phenocopy of Mgat5-/-TCR clustering. These data indicate that a galectin-glycoprotein lattice strengthened by Mgat5-modified glycans restricts T-cell receptor recruitment to the site of antigen presentation and lowers T-cell activation by directly enhancing T-cell receptor clustering.

Juvenile idiopathic arthritis is a heterogenous group of chronic inflammatory diseases of unknown etiology that is similar to rheumatoid arthritis. In patients with juvenile idiopathic arthritis there is hyperplasia of synovial cells and accumulation of mononuclear inflammatory infiltrates that are locally maintained through a balance between cell proliferation and apoptosis (62). Defective clearance of activated T-cells due to alterations in the Fas-Fas ligand system has been proposed, but this has not been confirmed in synovial tissue of patients with juvenile idiopathic arthritis. In patients with juvenile idiopathic arthritis the expression of pro-apoptotic galectin-1 was down-regulated and expression of galectin-3 was up-regulated (62). The study suggested that in patients with polyarticular (five or more joints are involved) juvenile idiopathic arthritis, there is an accumulation of inflammatory cells due to down-regulatation of apoptosis, and in patients with pauciarticular disease (less than five joints are involved) the process results from increased proliferation. Defective mononuclear apoptosis in synovial inflammatory infiltrates could be explained in part by decreased expression of galectin-1 and increased expression of galectin-3 (62).

Synovial tissues from patients with rheumatoid arthritis stained positively for galectin-3 messenger RNA and protein, and galectin-3 binding protein was expressed at sites of bone destruction. Galectin-3 was elevated in rheumatoid arthritis sera and synovial fluids and the serum levels correlated with C-reactive protein levels. The data indicate that galectin-3 and galectin-3 binding protein are involved in the inflammation of rheumatoid arthritis and in the activation of synovial fibroblasts (63).

Galectin-3 also is thought be enhance innate immunity and acute inflammation. Expression of galectin-3 activates mast cells, basophils (64) monocytes (55), and neutrophils (65). Descriptions of these cells can be found in a number of texts including The American Medical Association Encyclopedia of Medicine, Charles B. Clayman, MD, Medical Editor, Random House, New York, 1989; Dorland's Illustrated Medical Dictionary, 29th Edition, W. B. Saunders Company, Philadelphia, 2000; The Random House Dictionary of the English Language, Unabridged Edition, 1966; and Webster's Ninth New Collegiate Dictionary, 1991. Eosinophils have relatively large granules that take up red dye in routine stains and are more prevalent in both the circulation and at the site of inflammation in allergy and parasitic infections. The granules of eosinophils contain peroxidase, acid phosphatase, cationic major basic protein, and basic proteins that are toxic to certain parasites.

The neutrophil is the most numerous granulocyte, and has a relatively neutral color when its granules are stained. The neutrophil functions in cellular defense, becoming activated when exposed to proinflammatory mediators and chemotactic factors. Along with the macrophage, the neutrophil is termed a “professional” phagocyte, as a way of distinguishing these cell types from others that are able to ingest things, but is not able to ingest things to the same extent as other phagocytes.

Galectin-3 promotes the adhesion of human neutrophils to laminin (29). Recruitment of neutrophils from blood vessels to sites of infection represents one of the most important elements of innate immunity. Movement of neutrophils across blood vessel walls to the site of infection first requires that the migrating cells firmly attach to the endothelial wall. Generally, neutrophil extravasation is mediated at least in part by two classes of adhesion molecules, beta integrins and selectins (2). However, in the case of streptococcal pneumonia, recent studies have revealed that a significant proportion of neutrophil diapedesis is not mediated by the beta integrin/selectin paradigm (2).

Neutrophils express several heavily glycosylated carcinoembryonic antigen (CEA)-related glycoproteins (CD66 antigens). Galectin-3 has been shown to be a potent stimulus of human neutrophils if the receptor(s) for the lectin has been mobilized to the cell surface prior to activation. Neutrophil granules were used for isolation of galectin-3 receptors by affinity chromatography. Immunoblotting revealed these proteins were CD66a (160 kDa), CD66b (100 kDa), and lysosome-associated membrane glycoprotein-1 and -2 (Lamp-1 and -2; 120 kDa) (66).

Using an in vivo streptococcal pneumonia mouse model, accumulation of galectin-3 in the alveolar space of streptococcus-infected lungs was found to correlate closely with the onset of neutrophil extravasation. Immunohistological analysis of infected lung tissue revealed the presence of galectin-3 in the lung tissue areas composed of epithelial and endothelial cell layers as well as of interstitial spaces (67). In vitro, galectin-3 was able to promote neutrophil adhesion to endothelial cells. Promotion of neutrophil adhesion by galectin-3 appeared to result from direct cross-linking of neutrophils to the endothelium and was dependent on galectin-3 oligomerization. Together, these results suggest that galectin-3 acts as an adhesion molecule that can mediate neutrophil adhesion to endothelial cells (67).

Galectin-3 induces activation of NADPH oxidase in exudated but not in peripheral blood neutrophils (68). It appears that galectin-3 is a potent stimulus of the neutrophil respiratory burst, provided that the cells have first experienced an extravasation process (68). Superoxide production by neutrophils can be stimulated by galectin-3 (28) especially after being primed with lipopolysaccharide (69).

The spontaneous production of IgE by B cells from patients with IgE-associated atopic eczema/dermatitis syndrome is enhanced by polymorphonuclear leukocytes (PMN), but not by PMN from controls. The enhancement was abolished by preincubation of the PMN by lactose. Glucose and anti-galectin-3 serum, but not control rabbit serum, blocked the enhancement (59).

The least common leukocyte is the basophil that has many intracytoplasmic granules that stain intensely basophilic in routine preparations. Both basophils and mast cells express IgE receptors, making them potentially important in allergic responses. They are a rich source of vasoactive mediators, such as histamine, leukotrienes, platelet activating factor, and also produce neutrophil and eosinophil chemotactic factors. Mast cells are found in the tissues. Mast cells look like basophils, and like basophils have receptors for IgE, and produce proinflammatory mediators. However, mast cells are produced through a completely separate lineage than basophils.

Mononuclear phagocytes arise from hematopoietic stem cells in the bone marrow, and then, after passing through the monoblast and promonocyte states of the monocyte stage, circulate in the blood for about 40 hours. The mononuclear phagocytes then enter tissues and increase in size, phagocytic activity, and lysosomal enzyme content and become macrophages. The morphology of macrophages varies among different tissues and between normal and pathologic states. Not all macrophages can be identified by morphology alone. Most macrophages are large cells with a round or indented nucleus, a well-developed Golgi apparatus, abundant endocytotic vacuoles, lysosomes, and phagolysosomes, and a plasma membrane covered with ruffles or microvilli. Among the functions of macrophages are nonspecific phagocytosis and pinocytosis, specific phagocytosis of opsonized microorganisms mediated by Fc receptors and complement receptors, killing of ingested microorganisms, digestion and presentation of antigens to T and B lymphocytes, and secretion of a large number of diverse products, including many enzymes (lysozyme, collagenases, elastase, acid hydrolases), several complement components and coagulation factors, some prostaglandins and leukotrienes, and several regulatory molecules (interferon, interleukin-1). Among the cells now recognized as macrophages are histiocytes, Kupffer cells, osteoclasts, microglial cells, synovial type A cells, interdigitating cells, Langerhans cells (in normal tissues), epithelioid cells, and Langerhans-type and foreign-body-type multinucleated giant cells (in inflamed tissues).

Galectin-3 potentiates the lipopolysaccharide-stimulated production of interleukin-1 by monocytes (70) and induces chemotaxis of monocytes (57). Almost all murine microglial cells expressed galectin-3 intracellularly and about 90% expressed it on the cell surface (56). The expression of galectin-3 in normal human blood monocytes increases dramatically as the monocytes differentiate into macrophages in vitro. The level of expression was modulated by stimuli such as lipopolysaccharide, and interferon-γ. Soluble galectin-3 caused lactose-dependent superoxide release from human monocytes (55). Galectin-3 has also been found to be a chemoattractant factor for monocytes and macrophages (57).

Studies of mice deficient in galectin-3 due to targeted mutations in the galectin-3 gene provide definitive evidence of the pro-inflammatory activity of galectin-3. Compared with wild-type macrophages, galectin-3-deficient (gal3-/-) cells exhibited reduced phagocytosis of IgG-opsonized erythrocytes and apoptotic thymocytes in vitro. In addition, gal3-/-mice showed attenuated phagocytic clearance of apoptotic thymocytes by peritoneal macrophages in vivo. These mice also exhibited reduced IgG-mediated phagocytosis of erythrocytes by Kupffer cells in a murine model of autoimmune hemolytic anemia (71). In galectin-3-null-mutant mice there is a reduced number of peritoneal granulocytes (72) and lymphocytes (73), and peritoneal macrophages that normally express galectin-3 have an increased susceptibility to apoptosis (73).

Advanced glycation end products (AGE) are the reactive derivatives of nonenzymatic glucose-protein condensation reactions. This nonenzymatic glycation has been implicated in the multi-organ pathogenesis of the dysregulated tissue remodeling that characterizes diabetes and aging. An AGE-specific cellular receptor complex mediating AGE removal as well as multiple biological responses has been identified. Galectin-3 can serve in the assembly of the AGE-receptor complex components and in the efficient cell surface attachment and endocytosis by macrophages of a heterogenous pool of AGE moieties, thus contributing to the elimination of these pathogenic substances (74-76). It appears that galectin-3 plays an important role in formation of atherosclerotic lesions in vivo, by modulating endocytic uptake of AGE-proteins and modified low-density lipoproteins (77). Galectin-3 knockout mice that were rendered diabetic with streptozotocin were associated with a more marked renal/glomerular AGE accumulation, indicating AGE accumulation was attributable to the lack of galectin-3 AGE receptor function. The galectin-3-deficient genotype was associated with reduced expression of receptors implicated in AGE removal (macrophage scavenger receptor A and AGE-R1) and increased expression of those implicated in cell activation (RAGE and AGE-R2). These results show that the galectin-3-regulated AGE receptor pathway is operating in vivo but appears to protect toward some AGE-induced tissue injury in contrast to that through RAGE (78).

Compared with wild-type macrophages, galectin-3-deficient (gal3-/-) cells exhibited reduced phagocytosis of IgG-opsonized erythrocytes and apoptotic thymocytes in vitro. In addition, gal3-/-mice showed attenuated phagocytic clearance of apoptotic thymocytes by peritoneal macrophages in vivo. These mice also exhibited reduced IgG-mediated phagocytosis of erythrocytes by Kupffer cells in a murine model of autoimmune hemolytic anemia (71).

The regulatory mechanism that produces the variable localization of galectin-3 in different cell types is not understood, and the significance of the relative amounts of the protein found in the cytoplasm, nucleus, or extracellular matrix of various cell types in terms of functionality is not understood. Many laboratories have studied the role of galectin-3 in cancer, and although the results of the studies are somewhat confusing they do indicate that galectin-3 is significant in some types of cancer. However, the prior art is lacking in a methodology and a composition of galectin-3 that can be successfully used to treat cancer. It would therefore be useful to develop molecules, methods, and compositions based on an N-terminally truncated form of galectin-3 that inhibit multimerization and carbohydrate binding of galectin-3 and can be used successfully to reduce tumor growth and metastasis and other diseases.

### SUMMARY OF THE INVENTlON

According to the present invention, there is provided a composition having N-terminally truncated galectin-3, homologues of N-terminally truncated galectin-3, a nucleic acid sequence encoding N-terminally truncated galectin-3, or its homologues, in a pharmaceutically acceptable carrier. There is also provided a monoclonal or polyclonal antibody that specifically binds to carbohydrate ligands of galectin-3 in a pharmaceutically acceptable carrier.

Further, there is provided an anti-cancer or anti-inflammatory treatment having an effective amount of N-terminally truncated galectin-3 or its homologues, antibody to galectin-3 carbohydrate binding sites, or a nucleic acid sequence encoding N-terminally truncated galectin-3 or its homologues, in a pharmaceutically acceptable carrier.

Also provided is a method of treating disease by administering to a patient in need of such treatment an effective amount of N-terminally truncated galectin-3, its homologues, antibody to galectin-3 carbohydrate binding sites, or a nucleic acid sequence encoding N-terminally truncated galectin-3 in a pharmaceutically acceptable carrier.

### DESCRIPTION OF THE DRAWINGS

Other advantages of the present invention are readily appreciated, as the same becomes better understood by reference to the following detailed description when considered in connection with the accompanying drawings wherein:

FIG. 1 is a schematic showing a mechanism of action of N-terminally truncated galectin-3 whereby the truncated form of the protein inhibits the binding of intact galectin-3 to carbohydrate ligands and thereby also inhibits the multimerization and cross-linking activities of galectin-3;

FIG. 2 is a graph showing the number of cells that have bound to laminin in the presence of galectin-3, N-terminally truncated galectin-3 (galectin-3C), or a mixture of both galectin-3 and the N-terminally truncated galectin-3; the number of cells was determined by a standard curve for the absorbance at 450 nm due to metabolism of WST-1 by a known number of MDA-MB-435 cells;

FIG. 3 is a graph showing the toxicity of the therapy by plotting the weight change of the animals over time; FIG. 3A shows body weights of animals determined weekly during the course of the therapy study and data are presented as the mean and standard deviation of the body weights of the treatment and control groups; FIG. 3B shows body weight gains of animals exclusive of tumor weights at the termination of the therapy study, the body weights of the animals and their tumor weights were determined at the conclusion of the study and the weight of the tumor was subtracted from the final weight of the animal to calculate body weight gain; the black portion of the bar represents the mean body weights of mice in the treatment and control groups exclusive of tumor weights, and the gray portion of the bar represents the mean weights of tumors from mice in the treatment and control groups, the difference in body weight between the two groups of mice exclusive of tumor weight was not statistically significant (P=0.303);

FIG. 4 presents a pharmacokinetic analysis of the intramuscular administration of galectin-3C determined at 2-12 hours; mice were injected with ³⁵S-labeled galectin-3C and at the indicated time points the animals were sacrificed and serum and levels of radioactivity were determined; data are presented as the mean and standard deviation of the radioactive counts detected in five mice at each time point; the inset shows the pharmacokinetic analysis of the intravenous and intramuscular administration of galectin-3C was determined at eight hours; *p=0.033 when compared with cpm associated with blood cells (t-test);

FIG. 5 shows the organ biodistribution analysis of the subcutaneous and intravenous administration of galectin-3C; mice were injected with ³⁵S-labeled galectin-3C and at eight hours post injection the animals were sacrificed and the heart, lungs, liver, kidneys, and spleen were removed and the amount of associated radioactivity was determined; background radioactivity was determined by measuring radioactive counts associated with organs from mice injected with vehicle only; background counts for each organ were subtracted from the presented data; data are given as the mean and standard deviation of the percentage of the dose injected that was detected in the organs of five mice;

FIG. 6 is a graph of tumor volume over time showing the effect of galectin-3C treatment on the growth of primary orthotopic xenograph tumors in nude mice; mice were injected with either galectin-3C or vehicle control and the primary tumors were measured with calipers once per week after initiation of the treatment through the end of the 90 day treatment period; tumor volume was calculated by the formula W²×L/2, where W is the smallest dimension;

FIG. 7 is a bar graph showing the effect of galectin-3C treatment on the weight of primary orthotopic xenograph tumors in nude mice; mice were injected with either galectin-3C or vehicle control and the weight of the primary tumors was measured at the end of the 90 day treatment period;

FIG. 8 is graph of a repeated measures statistical model of the effect of galectin-3C treatment on the growth of primary orthotopic xenograph tumors in nude mice; a SAS Proc Mixed analysis was used to fit a linear mixed model with random effects for cage and mouse slope and intercept; the fitted model was quadratic on treatment day; the plot of the fitted model for log tumor volume versus treatment day shows steady growth for the control group but regressive growth for the galectin-3C group;

FIG. 9 is a graph showing the mean tumor volume versus the days of treatment with either the control, N-terminally truncated galectin-3, or galectin-3;

FIG. 10 is a series of photographs showing the efficacy evaluation of N-terminally truncated galectin-3 against the GFP-Gene Transfected Human Breast Cancer MDA-MB435 in a mouse model;

FIGS. 11 A-D are photographs showing a representative histopathology of primary tumor and lymph node, liver and lung metastasis in mice treated with the control;

FIGS. 12 A-D are photographs showing a representative histopathology of primary tumor and lymph node, lung, and liver metastasis in mice treated with the control;

FIGS. 13 A-C are photographs showing a representative histopathology of primary tumor and lymph node, and lung metastasis in mice treated with N-terminally truncated galectin-3;

FIGS. 14 A-C are photographs showing a representative histopathology of primary tumor, lymph node, and lung metastasis in mice treated with N-terminally truncated galectin-3;

FIGS. 15 A-C are photographs showing a representative histopathology of primary tumor, lymph node, and lung metastasis in mice treated with galectin-3;

FIGS. 16 A-E are diagrams of polyethylene glycol diol with two free hydroxyls, monomethoxypolyethylene glycol or mPEG with a single reactive hydroxyl, mPEG-succinimidyl propionate, that reacts with amino groups such as in lysine residues to produce stable amide linkages, and the sulfhydryl-selective PEGs, mPEG-maleimide and mPEG2-maleimide;

FIG. 17 is a diagram representing the reaction between the sulfhydryl on a cysteine residue of a protein and mPEG-maleimide;

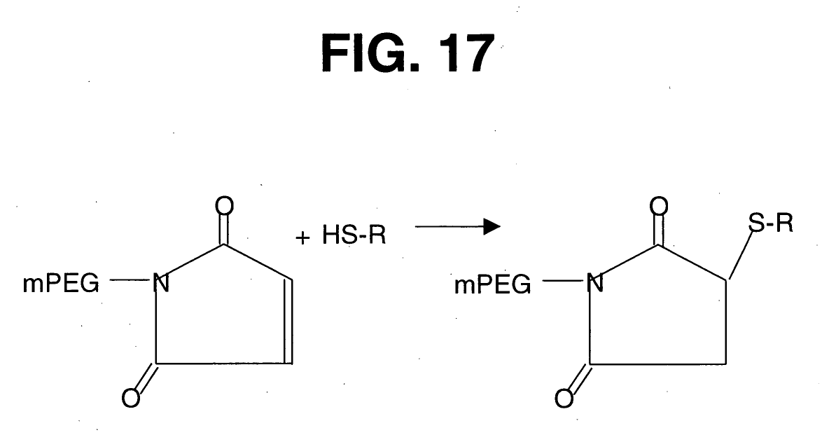

FIG. 18 is a diagram representing intact galectin-3 in a dimeric form;

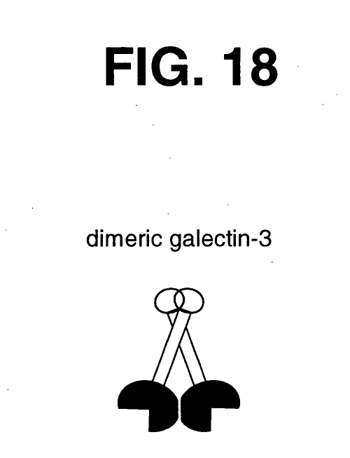

FIG. 19 is a diagram representing the monomeric form of N-terminally truncated galectin-3;

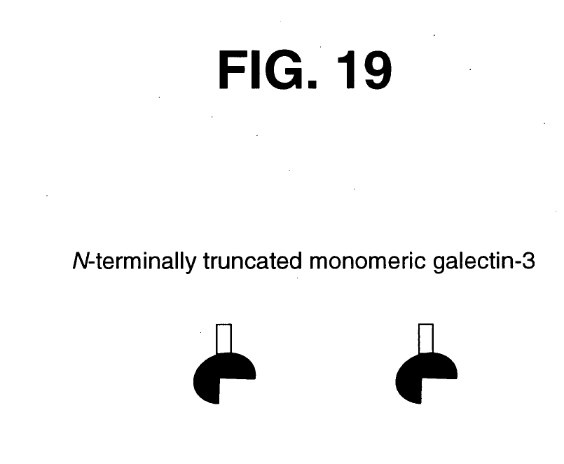

FIG. 20 is a schematic of the site-directed mutagenesis procedure for making a single amino acid substitution using a Site-Directed Mutagenesis QuikChange XL II kit (Stratagene, La Jolla, Calif.);

FIG. 21 is a schematic of the site-directed mutagenesis procedure for making up to three amino acid substitutions simultaneously using a Multi Site-Directed Mutagenesis QuikChange XL kit (Stratagene); and

FIG. 22 is a graph of fluorescence polarization (FP) analysis of two lots of N-terminally truncated galectin-3 made by Applicants by digestion with collagenase, the two lots show similar carbohydrate affinities.

### DETAILED DESCRIPTION OF THE INVENTION

The present invention provides a method and composition for treating cancer by administering an effective amount of N-terminally truncated galectin-3. Preferably, the N-terminally truncated galectin-3 is formulated for sustained or slow-release. The- N-terminally truncated galectin-3 is placed in a pharmaceutically acceptable carrier prior to administration. The present invention also provides a method and composition for treating diseases by administering an effective amount of an antibody that specifically binds to carbohydrate ligands of galectin-3.

The composition of the present invention includes a sequence encoding the N-terminally truncated galectin-3 and analogues and homologues thereof in a pharmaceutically acceptable carrier. The composition can be used as a gene therapy.

Compositions for treating cancer are provided by the present invention. There is provided a composition having N-terminally truncated galectin-3, with or without one or more cysteines added to the N-terminus, and that has been derivatized with PEG and that is in a pharmaceutically acceptable carrier. A composition is provided consisting of an antibody that binds to the complex saccharide ligands of galectin-3 and that is in a pharmaceutically acceptable carrier. Another composition provided by the present invention has N-terminally truncated galectin-3 that is derivatized with another chemical or formulated for sustained release and that is in a pharmaceutically acceptable carrier.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

It is noted that humans are treated generally longer than the mice or other experimental animals exemplified herein which treatment has a length proportional to the length of the disease process and drug effectiveness. The doses can be single doses or multiple doses over a period of several days, but single doses are preferred.

The doses can be single doses or multiple doses over a period of several days. The treatment generally has a length proportional to the length of the disease process and drug effectiveness and the patient species being treated.

Generally, the present invention provides a chemical entity, N-terminally truncated galectin-3, that binds lactose and that has been derivatized. The derivatization preferably occurs using one or more molecules of polyethylene glycol (PEG). The present invention provides a chemical entity, namely N-terminally truncated galectin-3, to which there has been added one or more cysteine residues that have been also derivatized. Again, the derivatization preferably occurs using one or more molecules of PEG. The N-terminally truncated galectin-3 and encapsulating pharmaceutically acceptable carrier has preferably been formulated for sustained release using methodologies that are well known to those skilled in the art.

Also provided by the present invention is a class of chemical entities that are antibodies that bind to complex saccharide ligands of galectin-3 and competitively inhibit the carbohydrate binding and, thereby, multimerization of galectin-3. The antibody and encasing pharmaceutically acceptable carrier has preferably been formulated for sustained release using methodologies that are well known to those skilled in the art.

The N-terminally truncated galectin-3 of the present invention is preferably lacking the N-terminal 107 amino acids. The N-terminally truncated galectin-3 was produced by enzymatic cleavage of the recombinant protein followed by collection of the pharmacologically active fragment, in the preferred method, and that carries PEG. Alternative methods of producing the N-terminally truncated galectin-3 can also be used such methods are known to those of skill in the art.

The N-terminally truncated galectin-3 proteins include truncated forms of the human recombinant galectin-3 protein designated SEQ ID NO. 3. This definition is not limited by the method(s) by which the proteins are obtained, and includes all the N-terminally truncated galectin-3 molecules otherwise within the definition, whether purified from nature source, obtained by recombinant DNA technology, synthesized, or prepared by any combination of these and/or other techniques known to those of skill in the art. Thus, there are provided N-terminally truncated human galectin-3 molecules that differ slightly in length. The molecules can be somewhat longer or shorter than the 143 amino acid residue N-terminally truncated galectin-3 that is lacking the N-terminal 107 amino acids. The molecules have essentially the same ability to inhibit the carbohydrate binding and multimerization of galectin-3 and, therefore, inhibit tumorigenicity and metastasis and other diseases in vivo, including, but not limited to inflammation resulting from arthritis and cancer.

The methodology for assessing the relative carbohydrate binding ability of proteins such as an N-terminally truncated galectin-3 can be performed by a variety of methods most of which are well known to those skilled in the art. The methods include, but are not limited to, methodsthat use lectin specific antibodies linked to enzymes for detection in an enzyme-linked immunosorbent assay (ELISA) format (25,79), labeling the protein directly with 3H or 1251 (11,25,80,81), or biotin (82) for detection, or by radio- or other labeling of the carbohydrate ligands (83) in binding assays. Other methods include chromatographic methods such as capillary electrophoresis and frontal affinity chromatography, and surface plasmon resonance (SPR) assays using a BlAcore system (BIAcore, Piscataway, N.J.). The BIAcore system enables the affinities of the binding interactions between a protein and its ligands to be calculated using the BIAevalution software.

Several studies have elucidated the portion of galectin-3 that is involved in carbohydrate recognition. The N-terminally truncated human galedtin-3 molecules that differ slightly in length also are derivatized with polyethylene glycol and are formulated for sustained or slow-release and placed in a pharmaceutically acceptable carrier.

The minimal lactose-binding domain of human galectin-3 was extensively mapped by use of a bacteriophage y surface expression vector (9). In a study by Seetharaman et al., (8), the detailed X-ray crystal structure of the carbohydrate recognition domain complexed with N-acetyllactosamine was disclosed. Such studies reveal the portions of intact galectin-3 (SEQ ID NO. 2) that are required for carbohydrate binding. The information can be used to help define the minimal portion of SEQ ID NO. 2 that is required for binding to carbohydrates. These portions of SEQ ID NO. 2 can be helpful to include in a molecule that can function as an inhibitor of galectin-3 for treatment of cancer.

Various studies have also examined the portion of galectin-3 that is required for homodimerization, cell cross-linking, hemagglutination, or homotypic aggregation and that make significant contribution to galectin-3 binding to carbohydrate ligands. These studies include those by Massa et al. (24), Hsu, et al. (26), Barboni et al. (84), and Ochieng et al. (85).

Massa et al. showed that the carbohydrate binding fragment of recombinant human galectin-3 that was produced by exhaustive digestion with collagenase type VII from Clostridium histiolyticum did not have cooperativity in carbohydrate binding but that the intact galectin-3 protein did have cooperativity (24). In another study Hsu et al. demonstrated that the product of digestion by collagenase enzyme from Achromobacter iophagus (Boehringer Mannheim) cleaved intact human galectin-3 at a site between Ala-111 and Gly-112. The C-terminal (Δ1-111) galectin-3 that retained carbohydrate binding failed to hemagglutinate rabbit erythrocytes, an activity possessed by wild type galectin-3 (26).

Ochieng et al. showed that a different Cterminal fragment of galectin-3 produced by digestion with a metalloproteinase enzyme also retained the ability to bind to carbohydrates but failed to homodimerize or hemagglutinate at concentrations at which the wild-type galectin-3 did homodimerize and hemagglutinate (85,86). The human metalloproteinase enzymes MMP-2 and MMP-9 cleaved wild type galectin-3 between Ala-62 and Tyr-63 producing two fragments, one containing part of the N-terminal domain and the other, (Δ1-62) galectin-3, containing the carbohydrate recognition domain (86).

Cell cross-linking, hemagglutination, homotypic aggregation, and increased cell anchorage occurs when galectin-3 is bound to carbohydrates on two different surfaces through dimerization produced by intermolecular binding of the galectin-3 N-terminal domains. Galectin-3 dimerization mediated by the N-terminal domain leads to cross-linking of cells with other cells or with the extracellular matrix. By removing portions of the N-terminal domain, as described above, dimerization of galectin-3, and thus its cross-linking ability, can be inhibited. Prevention of the cross-linking activity of galectin-3 also reduces its tumorigenicity, metastasis, and immunigenicity promoting activity.

It has been shown that N-terminally truncated galectin-3, (Δ1-111) galectin-3, can self-associate but this type dimerization can be completely inhibited by lactose (87). Dimerization mediated by Cterminal domain of galectin-3 does not enhance carbohydrate binding but instead occurs in competition with carbohydrate binding (87). In other words, C-terminal domain dimerization does not facilitate cell cross-linking, hemagglutination, homotypic aggregation, and cell anchorage.

The exact sequences of the set of the polypeptides based on SEQ. ID NO. 2 that can bind to carbohydrates- such as lactose but that do not demonstrate cooperativity in carbohydate binding, hemaglutination, and homotypic aggregation are defined by studies such as those cited above. The data provided herein establishes that N-terminally truncated galectin-3 polypeptides that possess these physical characteristics can be used to treat diseases including cancer by reducing tumorigenicity and metastasis. Thus, by correlating function with activity other polypeptide fragments of the structure shown in SEQ ID NO 2 can be created for use in treating cancer and other diseases. The treatment described herein uses any one or more of a set of polypeptides that includes amino acid sequences of SEQ ID NO 2 beginning with any of the amino acid residues from Tyr-63 through Arg-129, and that extends at least as far as any of the amino acid residues from Asp-241 through Ile-250. The carbohydrate binding ability of the N-terminally truncated galectin-3 proteins can be determined by various methods such as fluorescence polarization (88). Lack of ability of the protein fragments to induce homotypic aggregation of cancer cells expressing galectin-3 or hemagglutination of red blood cells can easily be determined to demonstrate lack of cross-linking ability (34,89).

The present invention provides N-terminally truncated galectin-3 molecules that contain fusion tags on the C or N-terminus. Examples of the tags include, but are not limited to, the commercially available His-Tag ® (Novagen, Madison, Wis.) for pET vector constructs that can contain 6, 8, or 10 histidine residues. These vectors can be used with more than a dozen different fusion tags that possess a variety of unique binding qualities and endoprotease sites that allow for high yields. A variety of methods, as are known to those of skill in the art, can be used to increase the purity of recombinant protein. The N-terminal tags can be cleaved off using enterokinase from Novagen, Roche (Indianapolis, Ind.) or Sigma (St. Louis, Mo.) or other similar compounds as are known to those of skill in the art. A C-terminal histidine₆ tag can be used for immobilized metal-affinity chromatography. C-terminal histidine₆ tag is not immunogenic, does not alter the affinity of most proteins, and has been used in both preclinical animal and human clinical trials (90). The methods for producing these slightly different versions of the N-terminally truncated human galectin-3 protein are known to those of skill in the art. Other pET vector expression systems are available from Novagen, such as pET3, which contain restriction enzyme sites that are used to produce the desired product without any fusion tags, can also be used in accordance with the present invention.

The present invention provides the C-terminal domain of galectin-3 for use as a therapeutic agent. Preferably, the therapeutic agent is modified to be in a slow release form.

Also provided by the present invention are antibodies that bind to the complex saccharide ligands of galectin-3 and that act to inhibit the carbohydrate binding and multimerization of galectin-3 as does the N-terminally truncated galectin-3.

The term “homologous” or “homology” refers to the extent of identity between the human galectin-3 and N-terminally truncated galectin-3 molecules at the amino acid level over the retained region of the sequence. Preferably, the N-terminally truncated protein has between 50-95% amino acid sequence homology with the galectin-3 protein, with a most preferred homology of at least 90%. The homology requirement is not stringent, however, the N-terminally truncated protein must retain the ability to bind carbohydrates but must not induce homodimerization or hemagglutination. In other words, the homology is sufficient as long as the N-terminally truncated protein binds and inhibits dimerization of intact (wild-type) galectin-3.

The N-terminally truncated protein and related functional homologues are useful for treatment and prevention of tumorigenicity, metastasis, and immunogenicity. The N-terminally truncated polypeptide of the invention includes conservative variations of the polypeptide sequence. The term “conservative variation” as used herein denotes the replacement of an amino acid residue by another, biologically similar residue.

Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

The truncated proteins of the present invention which “correspond to” a N-terminally truncated galectin-3 are, in general, homologous amino acid sequences (6) of SEQ ID NO: 1 including homologous in vitro generated variants having the qualitative biological activity defined herein.

The present invention provides an N-terminally truncated variant having at least the qualitative biological activity as defined herein and having, for example, at least about 75%, and preferably at least 90%, amino acid homology with the portions that it contains of the polypeptide of SEQ ID NO: 1. The variant amino acid sequence preferably shares at least 80%, more preferably, greater than 85% sequence homology with the portion that it contains of the sequence of SEQ ID NO: 1. However, a galectin-3 variant or related compound can exhibit less than 50% sequence homology with the sequence of SEQ ID NO: 1 and still retain the characteristics of a galectin-3 variant as described herein. In this regard, it is understood that amino acids can be substituted on the basis of side chain bulk, charge and/or hydrophobicity. Amino acid residues are classified into four major groups:
(i) Acidic: The residue has a negative charge due to loss of H ions at physiological pH and the residue is attracted by aqueous solution so as to seek the surface positions in the conformation of a peptide in which it is contained when the peptide is in aqueous solution.
(ii) Basic: The residue has a positive charge due to association with H ions at physiological pH and the residue is attracted by aqueous solution so as to seek the surface positions in the conformation of a peptide in which it is contained when the peptide is in aqueous medium at physiological pH.
(iii) Neutral/non-polar: The residues are not charged at physiological pH and the residue is repelled by aqueous solution so as to seek the inner positions in the conformation of a peptide in which it is contained when the peptide is in aqueous medium. These residues are also designated “hydrophobic residues.”
(iv) Neutral/polar: The residues are not charged at physiological pH, but the residue is attracted by aqueous solution so as to seek the outer positions in the conformation of a peptide in which it is contained when the peptide is in aqueous medium.
   Amino acid residues can be further classified as cyclic or non-cyclic, aromatic or non-aromatic with respect to their side chain groups these designations being commonplace to the skilled artisan.

### Original Exemplary Conservative Preferred Conservative

| Residue | Substitution | Substitution |
|---|---|---|
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala | Leu |
| Phe | Leu | Ile, Val |
| Ile | Met, Ala, Phe | |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Tyr |
| Tyr | Trp, Phe, Thr, Ser | Phe |
| Val | Ile, Leu, Met, Phe | Leu, Ala |

Commonly encountered amino acids that could be included in the N-terminally truncated protein but that are not encoded by the genetic code, include 2-amino adipic acid (Aad) for Glu and Asp; 2-aminopimelic acid (Apm) for Glu and Asp; 2-aminobutyric (Abu) acid for Met, Leu, and other aliphatic amino acids; 2-aminoheptanoic acid (Ahe) for Met, Leu and other aliphatic amino acids; 2-aminoisobutyric acid (Aib) for Gly; cyclohexylalanine (Cha) for Val, and

Leu and Ile; homoarginine (Har) for Arg and Lys; 2,3-diaminopropionic acid (Dpr) for Lys, Arg and His; N-ethylglycine (EtGly) for Gly, Pro, and Ala; N-ethylglycine (EtGly) for Gly, Pro, and Ala; N-ethylasparigine (EtAsn) for Asn, and Gin; Hydroxyllysine (Hyl) for Lys; allohydroxyllysine (AHyl) for Lys; 3-(and 4)hydoxyproline (3Hyp, 4Hyp) for Pro, Ser, and Thr; allo-isoleucine (AIle) for Ile, Leu, and Val; .rho.-amidinophenylalanine for Ala; N-methylglycine (MeGly, sarcosine) for Gly, Pro, and Ala; N-methylisoleucine (Melle) for Ile; Norvaline (Nva) for Met and other aliphatic amino acids; Norleucine (Nle) for Met and other aliphatic amino acids; Ornithine (Orn) for Lys, Arg and His; Citrulline (Cit) and methionine sulfoxide (MSO) for Thr, Asn and Gln; N-methylphenylalanine (MePhe), trimethylphenylalanine, halo (F, Cl, Br, and I) phenylalanine, triflourylphenylalanine, for Phe.

A useful method for identification of certain residues or regions of the N-terminally truncated galectin-3 variant for amino acid substitution other than those described herein is called alanine-scanning mutagenesis (91). Here a residue or group of target residues are identified (e.g. charged residues such as Arg, Asp, His, Lys, and Glu) and replaced by a neutral or negatively charged amino acid to affect the interaction of the amino acids with the surrounding aqueous environment in or outside the cell. Those domains demonstrating functional sensitivity to the substitution then are refined by introducing further or other variations at or for the sites of substitution. While the site for introducing an amino acid sequence variation is predetermined, the nature of the mutation per se need not be predetermined. For example, to optimize the performance of a mutation at a given site, Ala scanning or random mutagenesis, can be conducted at the target codon or region and the expressed galectin-3 variants screened for the optimal combination of desired activity.

Phage display of protein or peptide libraries offers another methodology for the selection of variants with improved affinity, altered specificity, or improved stability (92). High affinity proteins, displayed in a monovalent fashion as fusions with the M13 gene III coat protein (93), can be identified by cloning and sequencing the corresponding DNA packaged in the phagemid particles after a number of rounds of binding selection.

Proteins possess unique chemical and physical properties and the manufacture, formulation, and delivery of proteins represent significant challenges to pharmaceutical scientists (94). Proteins traditionally are administered by inconvenient modes such as intravenous injection or infusion, or subcutaneous injection and must be given frequently due to rapid degradation. Recent advances in clinically useful, sustained-release formulations for proteins allow less frequent administration. Examples of sustained-release formulation include, but are not limited to, injectable microspheres that encapsulate protein (95,96). One such product approved for human use, is manufactured by Nutropin Depot and is produced by encapsulating human growth factors in biodegradable microspheres. This formulation requires only one or two doses a month, but can require more than one injection per dose. Preparation of microspheres by lyophilization of a protein-PEG aqueous mixture has been reported for sustained release formulation (97). Polymers approved for use in humans for sustained release from biodegradable microsphere formulations include, but are not limited to, polylactides [poly(lactic acid), poly(glycolic acid), and poly(lactic-coglycolic) acid (98). Twice daily injections of the N-terminally truncated galectin-3 molecule were used in the mouse model of metastatic human breast described in Example 1. Formulation of the N-terminally truncated galectin-3 in a slow release formulation reduces the frequency of administration required and therefore increases the convenience of the method, increases the efficacy of the treatment by increasing the levels that could be sustained in the body by the treatment, and reduces the cost. Methods for making such formulations are known to those of skill in the art.

Many methods and PEG derivatives are available for conjugation of proteins to PEG (100-102). PEG modification of antibodies to tumors has been found to enhance penetration into the tumors and to increase the anti-tumor effects (103). The circulating lives of single chain antibodies have been extended by conjugation with PEG to the carboxylic acid groups or the primary amines. An increase in the polymer length rather than total mass was found to be more effective for serum half-life extension (104).

Specific PEG derivatives react with thiols such as the amino acid residue cysteine (101,105). These include PEG-ortopyridyl-disulphide, PEG-malemide, and PEG-vinylsulphone. In a preferred method, N-terminally truncated human galectin-3 that is lacking the 107 amino acids on the N-terminus, or is similar in function, is derivatized on the single cysteine in the sequence (SEQ ID NO. 1). Alternatively, the N-terminally truncated human galectin-3 can be produced with one or more cysteine residues on the N-terminus as described above (SEQ ID NO. 2). Then one or more of the cysteine residues is derivatized with a thiol reactive PEG derivative. Employing a branched PEG derivative and performing the derivatization in the presence of ligand can prevent active site residue derivatization due to steric hindrance (101).

The N-terminally truncated galectin-3 competitively inhibits the binding of galectin-3 to carbohydrates in the nucleus, cytoplasm, extracellular matrix and in cell-cell adhesions, and acts to prevent tumorigenicity and metastasis. The non-carbohydrate binding N-terminal domain of galectin-3 promotes multimerization of the protein, and enables it to cross link cancer cells to the matrix and other cells (24,25). The N-terminal half of the protein is the most critical for homophilic interactions, although there is evidence that the carbohydrate recognition domain can contribute to cross-linking (81,87,106).

Excess administered N-terminally truncated galectin-3, in which the carbohydrate binding part of the protein has been removed, occupies the binding sites of endogenous galectin-3. The N-terminally truncated galectin-3 itself has little or no cross-linking activity and acts as a dominant-negative inhibitor of galectin-3. Therefore, N-terminally truncated galectin-3 prevents the homophilic cross-linking of galectin-3 and other types of protein-protein binding interactions that promote tumorigenicity and metastasis.

Antibodies that bind to the carbohydrate ligands of galectin-3 also have this same effect. Studies of the specificity of galectin-3 have been performed (10,11,80,107). Galectin-3 has specific affinity for polyNAc-lactosaminoglycan, a polymer of β(1,3)-linked LacNAc units that occurs on cell surfaces and extracellular matrices. Oligosaccharides of the specific sequences recognized by galectin-3 can be conjugated and used to induce antibody production. The ability to produce antibody to oligosaccharide antigens is well established. This technique is used in immunotherapy using tumor-associated antigens to induce production by the patient's body of antibodies to tumors (108,109). Bispecific antibodies that are specific for a particular tumor type or tissue can be used to target the therapy (110-112). Generally, those of skill in the art know methodology for induction, isolation, and purification of both polyclonal and monoclonal antibodies.

The N-terminal domain is not required for oligosaccharide binding, but is necessary for positive cooperativity (24,26). The result of the positive cooperativity is that multimers of galectin-3 bind to substrate surfaces (24). Thus, galectin-3 multimers can cross-link a tumor cell with the extracellular membrane. The C-terminal fragment competitively inhibits the binding of galectin-3 to substrates.

More specifically, soluble recombinant N-terminally truncated galectin-3 effectively competes with endogenous galectin-3 for carbohydrate binding sites in the extracellular matrix and cell-cell adhesions important in tumor invasion and metastasis. The N-termihal domain of galectin-3 promotes multimerization of the protein, and enables it to cross-link cancer cells to the matrix and other cells. Excess administered N-terminally truncated galectin-3, in which the N-terminal part of the protein has been removed, occupies binding sites of endogenous galectin-3 and prevents its cross-linking activities. N-terminally truncated galectin-3 itself does not have significant cross-linking activity since it lacks the N-terminal part of galectin-3, and acts like a dominant-negative inhibitor of galectin-3.

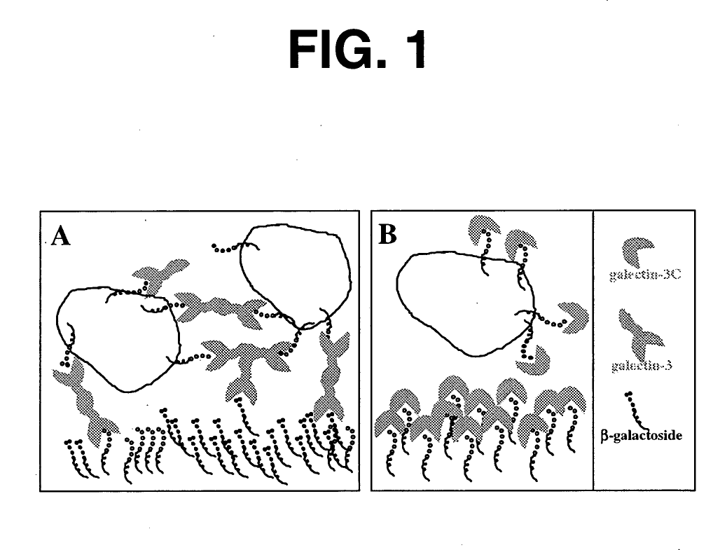

Recombinant N-terminally truncated galectin-3 is efficacious for inhibition of tumor invasion and metastasis in cancer. The mechanism shown in FIG. 1 is the competitive inhibition by recombinant galectin-3 of the binding of the galectin-3 on the surface of metastatic cancer cells to laminin and other galactoside glycoconjugates in the extracellular matrix. The N-terminal domain of galectin-3 promotes its multimerization and, thus, enables it to cross-link cancer cells to the matrix and to other cells. Excess administered truncated galectin-3, in which the N-terminal part of the galectin-3 has been removed, occupies the galectin-3 carbohydrate binding sites in the extracellular matrix and in cell-cell adhesions important in tumor invasion and metastasis. This truncated version of galectin-3 itself does not have cross-linking activity since it lacks the N-terminal domain of galectin-3. Hence, N-terminally truncated galectin-3 acts like a dominant-negative inhibitor of galectin-3 and prevents galectin-3 mediated binding of cells to the extracellular matrix and cell-cell adhesion as shown in FIG. 1. This concept is supported by the fact that galectin-3 itself, but not the N-terminally truncated galectin-3 produced by collagenase digestion, promotes binding of cells to laminin and fibronectin (29). The N-terminally truncated galectin-3 molecule, by blocking the multimerization of the intact protein, prevents the adhesion of tumor cells with one another and with the extracellular matrix. In addition to preventing adhesion to the extracellular matrix and, thus, metastasis, prevention of the contact of cancer cells with the extracellular matrix can also lead to their programmed cell death or apoptosis that is induced by loss of cell anchorage (also called anoikis) (49,50).

The amount of sustained release or PEGylated N-terminally truncated galectin-3 or antibody that is administered, which is utilized in the composition of the present invention, is present in a sufficient amount to at least reduce tumor size. Alternatively, the composition can prevent or reduce metastasis of a tumor. The tumors being treated can include breast cancer, prostate cancer, colon cancer, lung cancer, and all additional solid and liquid forms of cancer.

Alternatively, the compound of the present invention can be useful in preventing tumor growth. In such an example, by maintaining the titers of sustained release form or PEGylated N-terminally truncated galectin-3 or antibodies to galectin-3 saccharide in the body of an individual, the composition can prevent tumor growth by preventing tumors from forming in the first place. As stated in the Background Art, it is known that lectins could be useful in treating tumors and preventing metastasis, accordingly, by creating a preventative treatment that prevents the long term growth of tumor cells in the body, the compound of the present invention can be useful in preventing initial tumor growth. The amount of composition for treatment is based upon the body weight of the individual being treated and can be determined by individuals of skill in the art.

The present invention provides a method of treating cancer in a patient by administering to a patient in need of such treatment an effective amount of N-terminally truncated galectin-3 that has been derivatized with one or more molecules of polyethylene glycol (PEG). A second method of treating cancer in a patient is provided that includes administering to a patient an effective amount of N-terminally truncated galectin-3 to which there has been added one or more cysteine residues and that has been derivatized with PEG. A third method of treating cancer in a patient is provided that includes administering to a patient an effective amount of an antibody that binds to complex saccharide ligands of galectin-3. A fourth method of treating cancer in a patient is provided that includes administering to a patient an effective amount of N-terminally truncated galectin-3 that has been formulated for sustained release using methodologies that are well known to those skilled in the art.

The “effective amount” for purposes herein is thus determined by such considerations as are known in the art of cancer treatment wherein it must be effective to provide measurable improvement in persons given the treatment, and, in a preferred embodiment, complete recovery of the patient without the presence of cancer cells.

In order to effectuate the treatment of the present invention, there is administered to a patient an effective amount of the sustained release form of the N-terminally truncated galectin-3 or antibody to galectin-3 saccharide ligands in a pharmaceutically acceptable carrier. Administration can occur intramuscularly, orally, intravenously, locally, subcutaneously, or in any other applicable mechanism known to those of skill in the art. The mechanism of treatment varies depending upon the cancer that is being treated and can be best determined by those of skill in the art.

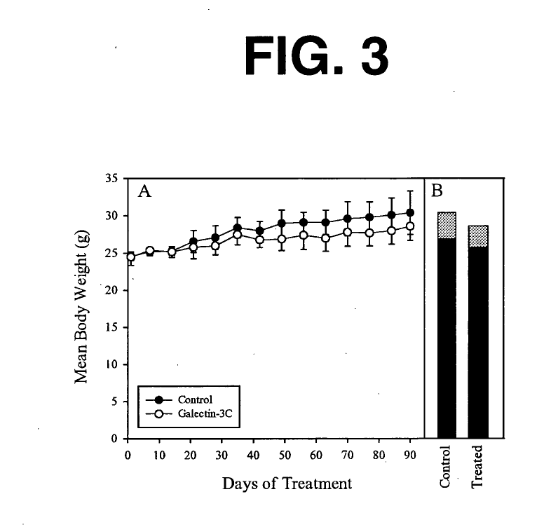

For example, N-terminally truncated galectin-3 was evaluated as a potential therapeutic agent for breast cancer based on the lectin galectin-3. It was determined that therapy with an N-terminally truncated form of galectin-3 is efficacious for inhibition of metastases. Recombinant galectin-3 was produced and the N-terminally truncated galectin-3 was derived by collagenase enzyme digestion and affinity chromatography. Injected N-terminally truncated galectin-3 was detected by metabolic labeling with ³⁵S methionine prior to collagenase cleavage. As shown in FIG. 3 the maximum tolerated dose of N-terminally truncated galectin-3 in nude mice was determined to be greater than 125 mg/kg without overt adverse effects. The pharmacokinetic elimination half-life of N-terminally truncated galectin-3 administered intramuscularly into nude mice was found to be 3.01 hours in the serum and 4.26 hours in the cellular fraction of the blood. Mice bearing orthotopically implanted tumors derived from breast cancer cell line MDA-MB435 was treated intramuscularly twice daily for 9.0 days with N-terminally truncated galectin-3 or a vehicle control. It was found that the mean tumor volumes and weights were statistically significantly less in mice treated with N-terminally truncated galectin-3 compared with control mice, and that fewer numbers of mice exhibited lymph node metastases in the treated group compared with the control group. It was therefore concluded that N-terminally truncated galectin-3 is not overtly toxic and is efficacious in reducing metastases and tumor volumes and weights in primary tumors.

Additionally, the compounds of the present invention can be utilized in a combination therapy. This can include adding to the pharmaceutically acceptable carrier additional chemotherapeutic compounds for further treatment of the cancer cells.

One method for producing the N-terminally truncated human galectin-3 includes first producing intact recombinant human galectin-3 that can be produced in Escherichia coli Bl21 (DE3) transformed with pET3cGal3 (the pET3c plasmid containing the human galectin-3 coding DNA) as described previously (24). Next, the bacteria can be lysed by sonication and the galectin-3 protein can be purified by affinity chromatography on lactosyl-Sepharose (24) and dialyzed to remove lactose. The product is then cleaved with Clostridium histiolyticum collagenase type VII (Sigma Chemical Co., St. Louis, Mo.). Specifically, the recombinant galectin-3 is incubated overnight at 37° C. with a lectin:collagenase ratio of 20:1 (by weight) in 20 mM Tris-HCl, pH 7.4, 150 mM NaCl, and 1 mM CaCl₂. The resulting N-terminally truncated galectin-3 can be purified by affinity chromatography on lactosyl-Sepharose. For storage and shipment, a procedure can be used involving dialysis against water followed by lyophilization. The dry N-terminally truncated galectin-3 powder can be stored at −20° C. for various amounts of time, up to three months, the time being determined by those of skill in the art. The retention of the carbohydrate binding activity of an aliquot can be ascertained by testing on a small lactosyl-Sepharose column. This establishes that the N-terminally truncated galectin-3 can be stored and shipped as a lyophilized powder without losing activity.

The amino acid sequence of the N-terminally truncated recombinant human galectin-3 that is produced by exhaustive digestion with collagenase, and that can be produced by other cloning methods is designated as SEQ ID NO. 1, and is as follows:

gap agplivpynl
plpggvvprm litilgtvkp nanrialdfq rgndvafhfn prfnennrrv ivcntkldnn
wgreerqsvf pfesgkpfki qvlvepdhfk vavndahllq ynhrvkklne isklgisgdi
dltsasytmi

The amino acid sequence of the N-terminally truncated recombinant human galectin-3 produced by cloning methods to contain one additional cysteine on the N-terminus is designated as SEQ ID NO. 2, and is as follows:

cgap agplivpynl
plpggvvprm litilgtvkp nanrialdfq rgndvafhfn prfnennrrv ivcntkldnn
wgreerqsvf pfesgkpfki qvlvepdhfk vavndahllq ynhrvkklne isklgisgdi
dltsasytmi

The amino acid sequence of the intact recombinant human galectin-3 described by Oda et al (7) is designated as SEQ ID NO. 3, and its sequence is as follows:

| | |
|---|---|
| 1 | madnfslhda lsgsgnpnpq gwpgawgnqp agaggypgas ypgaypgqap pgaypgqapp |
| 61 | gayhgapgay pgapapgvyp gppsgpgayp ssgqpsapga ypatgpygap agplivpynl |
| 121 | plpggvvprm litilgtvkp nanrialdfq rgndvafhfn prfnennrrv ivcntkldnn |
| 181 | wgreerqsvf pfesgkpfki qvlvepdhfk vavndahllq ynhrvkklne isklgisgdi |
| 241 | dltsasytmi |

A plasmid containing the complete galectin-3 coding sequence can be used as a template in a PCR reaction using primers designed to amplify the desired fragment.

Forward primer:
5′ GACGACGACAAGGGCGCCCCTGCTGGG 3′
Reverse primer:
5′ GAGGAGAAGCCCGGTTTATATCATGGTATA 3′

Underlined sequences in each of the primers match the plasmid sequences for pET32 (EK/LIC expression system, Novagen, Madison, Wis.). The reverse primer defines the C-terminal protein sequence and does not differ in these procedures. The non-underlined portion of the forward primer defines the N-truncated version of the native galectin-3 that begins with Gly-108 (Δ1-107″, starting at amino acid sequence glycine, alanine, proline, alanine, etc.). The underlined sequences are added as tails and are used to fuse the PCR product with the pET32 Ek/LIC plasmid using the Ek/LIC ligation protocol (Novagen, Madison, Wis.). The particular plasmid produces a fusion protein with a variety of unique binding qualities and endoprotease sites allowing for high yields and purity of the recombinant protein. More than one cysteine can be introduced to the construct by simply including more cysteine codons (either tgt or tgc) to create a version of N-truncated galectin-3 having one or more cysteines where they should not interfere with cabohydrate binding, for example, at the N- or C-terminus.

E. coli BL21(DE3) bacteria are transformed with the above-described construct and the bacteria can be used for protein production. Expression is under the control of bacteriophage T7 transcription and can be induced by providing a source of T7 RNA polymerase, such as infection with a phage that carries the T7 RNA polymerase gene or moving the plasmid into a cell containing an expression host containing a copy of the T7 RNA polymerase gene.

The above discussion provides a factual basis for the use of the compositions of the present invention. The method used with and the utility of the present invention can be shown by the following non-limiting examples and accompanying figures.

### EXAMPLES

General methods in molecular biology: Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences. (113).

General methods in immunology: Standard methods in immunology known in the art and not specifically described are generally followed as in Stites et al.(eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

Immunoassays

In general, ELISAs are the preferred immunoassays employed to assess a specimen. ELISA assays are well known to those skilled in the art. Both polyclonal and monoclonal antibodies can be used in the assays. Where appropriate other immunoassays, such as radioimmunoassays (RIA) can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, Molecular Cloning: A Laboratory Manual, Cold Springs Harbor, N.Y.,1989.

Antibody Production

Antibody Production: Antibodies can be monoclonal, polyclonal or recombinant. Conveniently, the antibodies can be prepared against the immunogen or portion thereof for example a synthetic peptide based on the sequence, or prepared recombinantly by cloning techniques or the natural gene product and/or portions thereof can be isolated and used as the immunogen. Immunogens can be used to produce antibodies by standard antibody production technology well known to those skilled in the art as described generally in Harlow and Lane, Antibodies: A Laboratory Manual, Cold Spring Harbor Laboratory, Cold Spring Harbor, N.Y., 1988 and Borrebaeck, Antibody Engineering—A Practical Guide, W.H. Freeman and Co., 1992. Antibody fragments can also be prepared from the antibodies and include Fab, F(ab′)₂, and Fv by methods known to those skilled in the art.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific.

For producing monoclonal antibodies the technique involves hyperimmunization of an appropriate donor with the immunogen, generally a mouse, and isolation of spleenic antibody producing cells. These cells are fused to a cell having immortality, such as a myeloma cell, to provide a fused cell hybrid that has immortality and secretes the required antibody. The cells are then cultured, in bulk, and the monoclonal antibodies harvested from the culture media for use.

For producing recombinant antibody (see (114,115), and Mernaugh and Mernaugh, 1995), messenger RNAs from antibody producing B-lymphocytes of animals, or hybridoma are reverse-transcribed to obtain complimentary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. (For a general discussion of conjugation of fluorescent or enzymatic moieties see Johnstone & Thorpe, Immunochemistry in Practice, Blackwell Scientific Publications, Oxford, 1982.) The binding of antibodies to a solid support substrate is also well known in the art (see for a general discussion Harlow & Lane Antibodies: A Laboratory Manual, Cold Spring Harbor Laboratory Publications, New York, 1988 and Borrebaeck, Antibody Engineering—A Practical Guide, W.H. Freeman and Co., 1992). The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹⁴C and iodination.

Delivery of Gene Products/Therapeutics (Compound):

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein that treatment has a length proportional to the length of the disease process and drug effectiveness. The doses can be single doses or multiple doses over a period of days or weeks.

When administering the compound of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives that enhance the stability, sterility, and isotonicity of the compositions, including anti-microbial preservatives, anti-oxidants, chelating agents, and buffers, can be added. Prevention of the action of microorganisms can be ensured by various anti-bacterial and anti-fungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used is compatible with the compounds.

Sterile injectable solutions can be prepared by incorporating the compounds utilized in practicing the present invention in the required amount of the appropriate solvent with several of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques that deliver it orally or intravenously and retain the biological activity are preferred.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered varies for the patient being treated and varies from about 100 ng/kg of body weight to 100 mg/kg of body weight per day and preferably is from 10 mg/kg to 10 mg/kg per day.

### Example 1

As background for the following example, mounting evidence suggests that tumor cells express the β-galactoside-binding lectin galectin-3 on their surfaces and that tumor cells metastasize partly due to processes involving cellular adhesion and aggregation mediated by galectin-3. Galectin-3 binds via its C-terminus carbohydrate recognition domain to binding sites in the extracellular matrix. The goal of this research was the evaluation of a potential therapeutic agent for breast cancer based on galectin-3 lectin that acts directly to reduce metastases. Soluble recombinant N-terminally truncated galectin-3 competes with endogenous galectin-3 for carbohydrate binding sites in the extracellular matrix and cell-cell adhesions important in tumor invasion and metastasis. The N-terminal domain of galectin-3 promotes multimerization of the protein, and enables it to cross link cancer cells to the matrix and other cells. Excess administered N-terminally truncated galectin-3, in which the N-terminal part of the protein has been removed, occupies binding sites of endogenous galectin-3 and prevents its cross-linking activities. N-terminally truncated galectin-3 itself has less cross-linking activity since it lacks the N-terminal part of galectin-3, and acts like a dominant-negative inhibitor of galectin-3, as shown in FIG. 1. The experiments establish that therapy with recombinant N-terminally truncated galectin-3 is efficacious for inhibition of tumor invasion and metastasis in breast cancer. The overall purpose was to determine the efficacy, safety, and mechanism of action of N-terminally truncated galectin-3 in treatment of metastatic breast cancer using a nude mouse model of metastasis.

Preparation for Animal Studies.

The carbohydrate recognition domain of galectin-3 (N-terminally truncated galectin-3) was produced as described previously (24). High yield expression (20-300 mg/liter culture) of active soluble galectin-3 can be obtained. The intact recombinant galectin-3 was produced in Escherichia coli Bl21/DE3 containing the pET3c plasmid (Novagen) with the human galectin-3 coding DNA (pET3cGal3). Galectin encoding DNA can be amplified by PCR using galectin cDNA as template and primers that contain restriction sites for cloning of the product into the expression vector in the proper reading frame. The PCR product is first cloned into the TA-vector (Invitrogen, Carlsbad, Calif.), the insert is released with the restriction enzymes corresponding to the primers, and it is finally cloned into the expression vector. The expression construct is used to transform the proper host strain (e.g. E. coli BL21 for the pET system). The organisms are lysed by sonication and the galectin-3 protein is purified by affinity chromatography on lactosyl-Sepharose (24). The purified galectin-3 is dialyzed to remove lactose and cleaved with Clostridium histiolyticum collagenase type VII (Sigma). The resulting N-terminally truncated galectin-3 is purified again by affinity chromatography on lactosyl-Sepharose.

For storage and shipment a new procedure was developed involving dialysis against water followed by lyophilization. The dry N-terminally truncated galectin-3 powder was stored at −20° C. for various amounts of time up to three months and the retention of the carbohydrate binding activity of an aliquot was ascertained by testing on a small lactosyl-Sepharose column. Other batches (with or without enrichment in ¹⁵N) were analyzed by NMR-spectroscopy. This analysis confirmed that the protein had retained its proper folding. Therefore, N-terminally truncated galectin-3 can be stored and shipped as a lyophilized powder without loosing activity.

To produce ³⁵S labeled N-terminally truncated galectin-3 for pharmacokinetic studies, the plasmid pET3cGal3 was transfected into E. coli B834 (Novagen), which is a methionine auxiotroph derived from BL21/DE3. The E. coli was adapted for growth on M9 minimal medium supplemented with ampicillin (50 mg/ml) and methionine (40 mg/ml)(M9-Met) by passage on M9-Met plates three times. To produce ³⁵S galectin-3, a colony from the last plate was inoculated into 0.5 liters of M9-Met supplemented with 1.0 mCi ³⁵S-Met. The bacteria were cultured, induced with IPTG and harvested as described previously (24). To lyse the radioactive E. coli, sonication was avoided because of aerosol formation. Various alternative methods were tested and the following method was determined to be most efficient. To the bacterial pellet there was added 5 ml sucrose (25%) in 50 mM TrisHCl, pH 8.0 with 50 mM NaCl, 20 mM EDTA, and 8 mg lysozyme. After ten minutes on ice, 16 ml water was added and the sample kept on ice another 30 minutes. The sample was centrifuged at 12000 rpm for 30 minutes and the supernatant applied to lactosyl-Sepharose. The galectin-3 was eluted, dialyzed and treated with collagenase to generate N-terminally truncated galectin-3 as described above.

Cellular Adhesion Assay

A cellular adhesion assay was performed that is similar to that described previously (29) for use in 96-well microtiter plates. The wells were coated with 50 microliters of 20 micrograms/ml of human laminin in PBS overnight at 4° C. The wells then were washed once with minimum essential media (MEM), and then blocked for 1 hour at 37° C. with 1% R.I.A. grade BSA in MEM, washed once with MEM containing 0.1% Tween-20 and twice with MEM. To each well was added 4.5×10⁴ human breast cancer cells, MDA-MB435 with various concentrations of galectin-3, the N-terminally truncated galectin-3, or a mixture of both proteins. Either 0, 5, 10, 15, or 20 micrograms per ml of galectin-3 or the N-terminally truncated protein were added in MEM. In a third set of wells, 20 micrograms per ml of N-terminally truncated galectin-3 was added with 5, 10, 15, or 20 micrograms per ml of galectin-3 in MEM. After incubation for 15 minutes at 37° C. the nonadherent cells were removed and the wells gently washed with MEM. To each well was added 100 microliters of MEM and 10 microliters of WST-1 cell viability reagent (Roche, Mannheim, Germany) and the plate was placed in a CO₂ incubator at 37° C. for 90 minutes. The UV absorbance of the samples was recorded at 450 nm subtracting the absorbance at 650 nm in a Molecular Devices Thermomax plate reader.

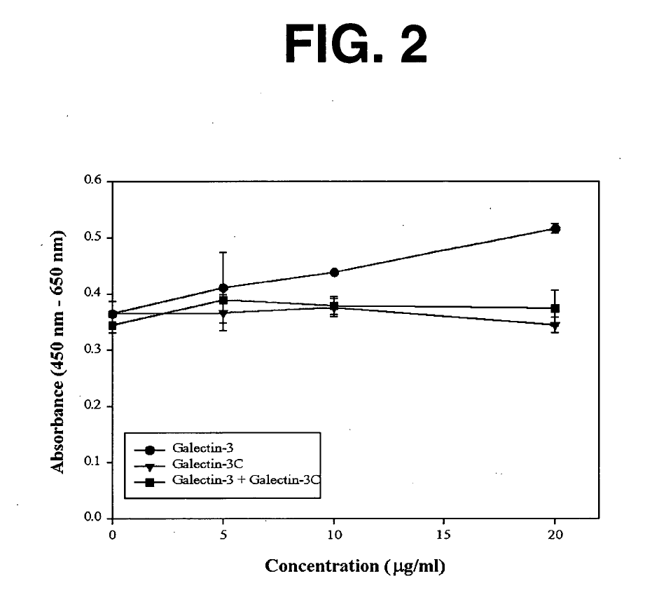

The results of the assay are shown in FIG. 2. The increased number of cells adherent to laminin with increasing concentrations of galectin-3 is clearly revealed. Similarly the lack of significant effect of increasing concentrations of N-terminally truncated galectin-3 is also revealed and is similar to the previously reported data (29). Adding the N-terminally truncated galectin-3 blocked the increase in cellular adhesion to laminin mediated by galectin-3. The data show the directly competitive effect of the N-terminally truncated protein on the cellular adhesion that is promoted by intact galectin-3, and supports the mechanism proposed in FIG. 1.

Immunization of four chickens with purified, N-terminally truncated galectin-3 and purification of polyclonal Ig from chicken eggs.

Two chickens were immunized with purified N-terminally truncated galectin-3 and the polyclonal Ig was purified. The Ig should bind to both galectin-3 and N-terminally truncated galectin-3. When tested, the chicken polyclonal anti-N-terminally truncated galectin-3 was of very low affinity as determined by repeated nitrocellulose dot blots of galectin-3 and N-terminally truncated galectin-3 following lactose elution of each protein from a lactosyl-Sepharose column. The presence of galectin-3 and N-terminally truncated galectin-3 protein in specific fractions from the column was confirmed by the measurement of UV absorbance at 280 nm. Detection of anti-galectin-3 antibody (rat IgG) binding to galectin-3 was used as a positive control on a separate dot blot using anti-rat IgG labeled with alkaline phosphatase (AP). For the chicken polyclonal anti-N-terminally truncated galectin-3 antibody, an anti-chicken Ig antibody (Zymed, South San Francisco) labeled with biotin was used followed by AP-conjugated streptavidin and AP substrate. The results of these studies provided no evidence that immunization of chickens produces a high affinity antibody specific for N-terminally truncated galectin-3. Therefore, an alternative strategy of generating ³⁵S-labeled N-terminally truncated galectin-3 was used.

Pharmacokinetic Analysis & Determination of Maximum Tolerated Dose.

A dose determination study was carried out in non-tumor bearing female athymic nude mice in order to establish the MTD of N-terminally truncated galectin-3 using a single bolus dose. The dose finding study comprised four dose groups with each group consisting of five mice. The subcutaneous doses administered were 1 mg/kg, 5 mg/kg, 25 mg/kg, and 125 mg/kg. In addition, a vehicle treated control group consisting of five mice was evaluated. No overt abnormal signs were observed within 48 hours of injection. Animals were observed for a total of five days after injection at which time body weight and viability were determined. The mean body weights for each group were statistically identical (p>0.10; t-test) at five days indicating that all doses of N-terminally truncated galectin-3 did not effect the normal physiological growth of the mice. From these results it was concluded that N-terminally truncated galectin-3 can be injected into nude mice at a dose as high as 125 mg/kg without overt adverse effects.

Pharmacokinetic Analysis.

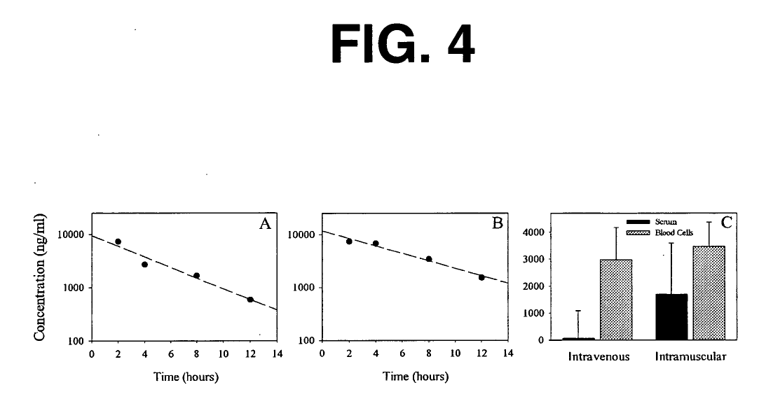

Analyses of the pharmacokinetic and biodistribution characteristics of N-terminally truncated galectin-3 were determined for intravenous and intramuscular routes of administration of N-terminally truncated galectin-3 into nude mice as shown in FIG. 4. Groups of five mice (approximately 0.03 kg/mouse) were each injected with 150 μg/mouse (1 mg per ml; 5 mg/kg=dose) of a mixture of ³⁵S-labeled N-terminally truncated galectin-3 and unlabeled N-terminally truncated galectin-3 in a weight ratio of 1:9 (labeled:unlabeled). For the intramuscular route, the animals were sacrificed and blood samples were obtained by terminal cardiac puncture at four time points: 2 hours, 4 hours, 8 hours, and 12 hours after injection. In addition, blood samples were obtained from one control group of five animals 1 hour after injection of vehicle only (1 mg/ml lactose in PBS). Serum samples from 200 μl of blood were analyzed for radioactivity in triplicate. For a direct comparison of the pharmacokinetic characteristics of intravenous versus intramuscular administration of N-terminally truncated galectin-3, two groups of five mice each were injected with the mixture of ³⁵S-labeled N-terminally truncated galectin-3 and unlabeled N-terminally truncated galectin-3 either intravenously or intramuscularly. At eight hours post-injection serum and blood cell samples from 200 μl of blood were analyzed for radioactivity in triplicate. In addition, the organ biodistribution of N-terminally truncated galectin-3 was determined in mice injected subcutaneously and intravenously with the mixture of ³⁵S-labeled N-terminally truncated galectin-3 and unlabeled N-terminally truncated galectin-3. At eight hours post-injection, the heart, lungs, liver, kidneys, and spleen were removed and the amount of associated radioactivity was measured.

During the distribution phase after an intravenous dose, changes in the concentration of drug are primarily due to movement of drug within the body. The distribution phase primarily determines the early rapid decline in plasma concentration of a drug after an intravenous dose. With time, equilibrium is reached in the distribution of the drug between the plasma and the tissues, and changes in plasma reflect proportional changes in all the other tissues. During the elimination phase, after the rapid decline of the distribution phase, the decline in plasma concentration is due only to elimination of the drug from the body and is characterized by the elimination half-life (T½) (116,117). The elimination half-life is the time it takes for the concentration of the drug in the plasma (and body) to be reduced by one-half. The apparent volume of distribution is the apparent volume of distribution of the drug in the body at equilibrium. The volume of distribution is equal to the amount of drug in the body at T₀ divided by the plasma drug concentration at T₀.

In a first-order elimination process the half-life is independent of the concentration of the drug in the body and the following equations apply.

$$\begin{matrix}Equation 1. & {T}_{1 / 2} = \frac{0.693}{k} ( where k is the elimination rate constant ) \\ Equation 2. & k = \frac{2.303}{Time ( 2 ) - Time ( 1 )} \times log \frac{{conc}_{Time ( 1 )}}{{conc}_{Time ( 2 )}} \end{matrix}$$

Calculation of the volume of distribution requires that distribution equilibrium be achieved between the drug in the tissues and the plasma. After administration, the amount of the drug in the body is equal to the dose but the distribution equilibrium has not yet been achieved. To estimate the plasma concentration that would have resulted if the drug had been immediately distributed into its final volume of distribution, the linear decline during the elimination phase, as shown in the semilogarithmic plot, is used.

The following formula based on the exponential decay law was used to calculate the amount of radioactivity present in the galectin-3C at the time of injection based on the specific activity previously determined per μg of protein.
ln N/N₀=−λt Equation 3.

In this formula N is the number of undecomposed atoms at elapsed time t, N⁰ is number of atoms originally present (when t=0), and λ is the decay constant. The half-life (T½) of ³⁵S that is 87.2 days was used to characterize the rate of decay (λ=0.693/T½).

The pharmacokinetic analysis of the intramuscular administration of N-terminally truncated galectin-3 is shown in FIG. 4. The serum elimination half-life (FIG. 4A) and the elimination half-life of the cellular fraction of the blood (FIG. 4B) were calculated by regression analysis of the linear portion of the curve between 2 and 12 hours as described above. Thus, for the intramuscular administration of N-terminally truncated galectin-3, the serum T½=3.01 h and the cellular fraction T½=4.26 h.

The eight hours of distribution of N-terminally truncated galectin-3 into serum versus blood cell compartments for N-terminally truncated galectin-3 administered intravenously and intramuscularly was compared. As shown in FIG. 4C, both routes of administration resulted in the distribution of N-terminally truncated galectin-3 into serum and blood cells. For intravenous administration, the quantity of N-terminally truncated galectin-3 associated with blood cells was greater than that in serum (p=0.03; t-test). For intramuscular administration, there was not a statistically significant difference in the amount of N-terminally truncated galectin-3 localized to either compartment. These findings indicate that a portion of the administered N-terminally truncated galectin-3 is associated with blood cells and can serve as a reservoir for slow release in serum.

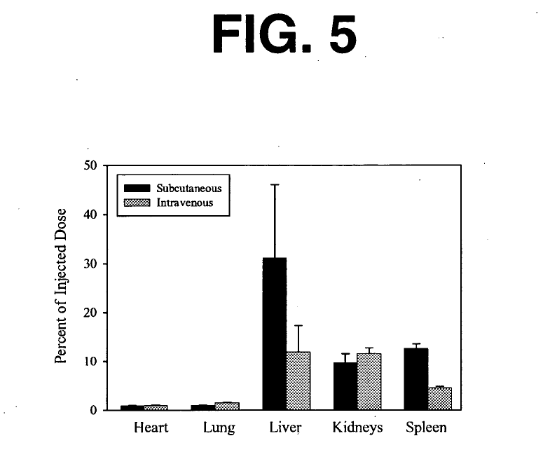

The organ biodistribution of N-terminally truncated galectin-3 was also investigated. Two groups of five mice were injected either subcutaneously or intravenously with the mixture of ³⁵S-labeled N-terminally truncated galectin-3 and unlabeled N-terminally truncated galectin-3. A third group of five mice was injected intravenously with the lactose in PBS vehicle. At eight hours post-injection, the heart, lungs, liver, kidneys, and spleen were removed and the amount of associated radioactivity was measured. As shown in FIG. 5, the liver, kidney, and spleen were sources of N-terminally truncated galectin-3-associated radioactivity above the background vehicle level, whereas neither the heart nor lungs localized any N-terminally truncated galectin-3. These data suggest that in addition to serum and blood cells, certain organs can localize N-terminally truncated galectin-3 and possibly serve as reservoirs for eventual systemic release. Alternatively, these organs can function in the ultimate removal of N-terminally truncated galectin-3 from circulation.

Comparison in the MetaMouse^{R} Model of Metastatic Breast Cancer of the Efficacy of Treatment with N-terminally Truncated Galectin-3 to Control Animals (Vehicle Only).

Female athymic CD-1 nude mice between four and five weeks of age were used in the study. The animals were bred and maintained in a HEPA-filtered environment with cages, food, and bedding sterilized by autoclaving. The breeding pairs were obtained from the Charles River Laboratories (Wilmington, Mass.). The animal diets were obtained from Harlan Teklad (Madison, Wis.). Ampicillin (Sigma) at a concentration of 5% (v/v) was added to the autoclaved drinking water.

Breast cancer cell line MDA-MB435, that expresses galectin-3 (118), was transfected with a plasmid expressing green fluorescent protein as previously described (119), and cells were injected into the subcutis of nude mice to form solid tumors. Test animals for the study were transplanted by surgical orthotopic implantation using fragments harvested from the subcutaneously growing tumors. The animals were anesthetized with isoflurane and the surgical area was sterilized using iodine solution and alcohol. An incision approximately 0.5 cm long was made on the second right mammary gland. The gland was then pulled out and two fragments of 1 mm³ of MDA-MB435-GFP tumor tissue were sutured onto the gland with a sterile nylon 8-0 surgical suture. The skin incision was closed with a sterile silk 6-0 surgical suture. All surgical and animal manipulations and procedures were conducted under HEPA-filtered laminar flow hoods.

Extra numbers of mice were transplanted to compensate for possible postsurgical losses and tumor non-takes. The orthotopically-transplanted animals used for the study were selected to establish groups of similar mean tumor size and body weight. Groups for each of the cohort conditions were randomly chosen. The cohort study was grouped as shown in Table 1 for a total of 45 mice. Administration of the treatments was begun when tumors reached palpable sizes. Based on the calculated elimination half-life of N-terminally truncated galectin-3 administered intramuscularly, the dosing schedule for the vehicle control and N-terminally truncated galectin-3 groups was twice a day for the intramuscular injections with an approximately 6-8 hour interval between injections for a total 90 days. The dosing for galectin-3 was once a day intramuscular injections. Both N-terminally truncated galectin-3 and galectin-3 were injected as solutions of concentration 1 mg/ml in PBS containing 1 mg/ml lactose. The vehicle control was PBS containing 1 mg/ml lactose.

TABLE 1
Efficacy Test Study Design

| | | | Number of |
|---|---|---|---|
| Group | Dose | Dosing Schedule | Mice |
| Vehicle Control | 125 μl/dose | im, bid × 90 days | 20 |
| N-terminally truncated | 125 μg/dose | im, bid × 90 days | 20 |
| galectin-3 | | | |
| Galectin-3 | 110 μg/dose | im, qd × 90 days | 5 |

Data Collection and Results:
Body weight and animal survival—Animal weight was determined by an electronic balance once a week during the course of the efficacy test. FIG. 3 shows the mean body weights of the mice over time in each of the three treatment groups. The mean body weight of the mice in the control group at day 90 was 30.4±2.89 g, whereas that of the N-terminally truncated galectin-3 treatment group was 28.6±1.91 g. This represents a statistically significant difference in the body weights between the two groups (p=0.026; t-test). The likely explanation for this finding is that the control group had tumors that weighed more than the tumors in the N-terminally truncated galectin-3 group as described below (tumor weight: control mean 2.34 g vs. treated mean 1.25 g). The tumor-free mean body weight changes of the test and control groups were statistically identical (p=0.303). In addition, the difference in body weights between the control group and the galectin-3 treated group was not statistically significant.

All animals in all three groups survived the 90-day course of treatment. Thus, no overt toxicity was observed for treatment of mice with either N-terminally truncated galectin-3 or galectin-3 for a period of 90 days.

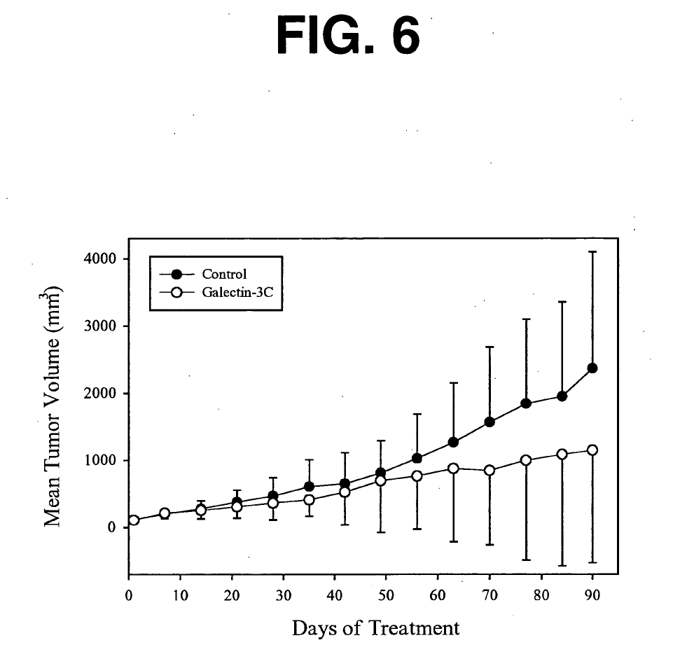

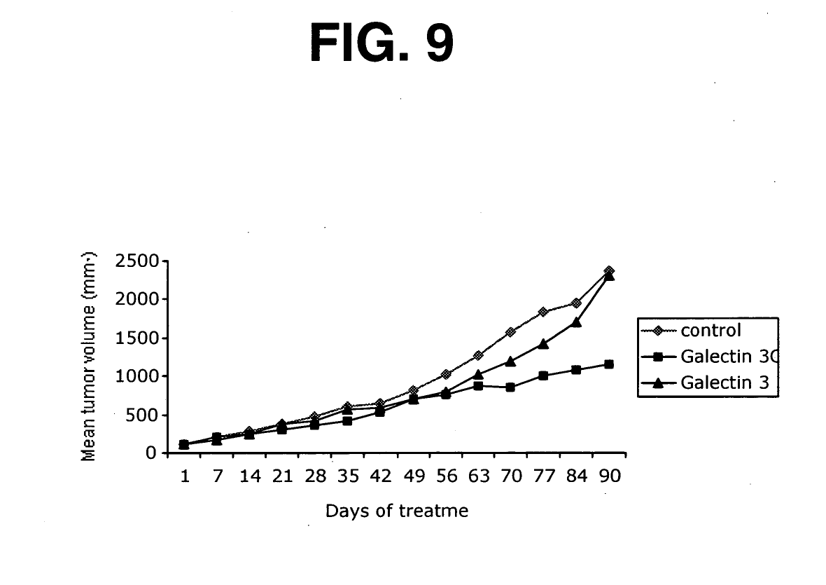

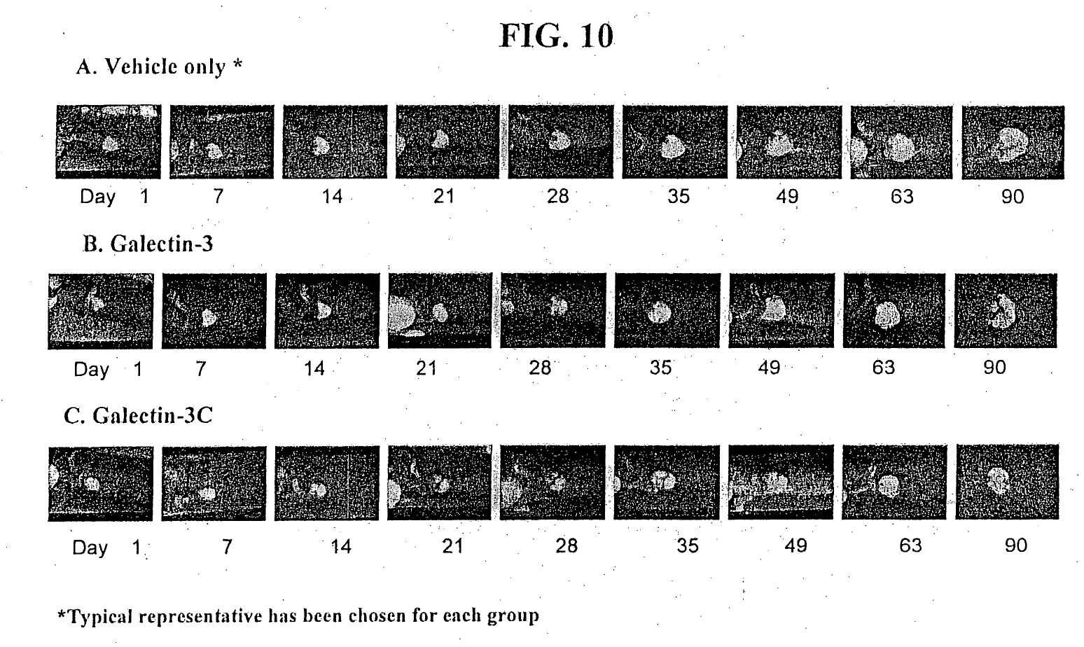

Primary tumor volume and weight—The primary tumors were measured by a pair of calipers once a week from initiation of treatment through the end of the study. The mean tumor volume over time for galectin-3C compared to the vehicle-only control group is shown in FIG. 6, and the galectin-3 treated group is included in FIG. 9. Table 2 shows a comparison of the mean tumor volumes of the mice in each group at the end of the treatment regime. The mean tumor volume measured in the mice treated with N-terminally truncated galectin-3 was significantly less than that in the control mice (p=0.003), whereas the mean tumor volume in mice treated with galectin-3 was similar to that in the control mice (p=0.865). Using a whole-body optical imaging system, the growth of the GFP-expressing tumors was also visualized in real time. FIG. 10 shows imaging analyses of the 90-day efficacy test of N-terminally truncated galectin-3. Tumor fragments of breast cancer cell line MDA-MB-435 expressing green fluorescent protein were orthotopically implanted into the breast pad of nude mice. Real time, quantitative measurement of tumor growth, metastases, and micrometastases were performed using whole-body optical imaging. The images are representative of the external images of the development of the breast tumor.

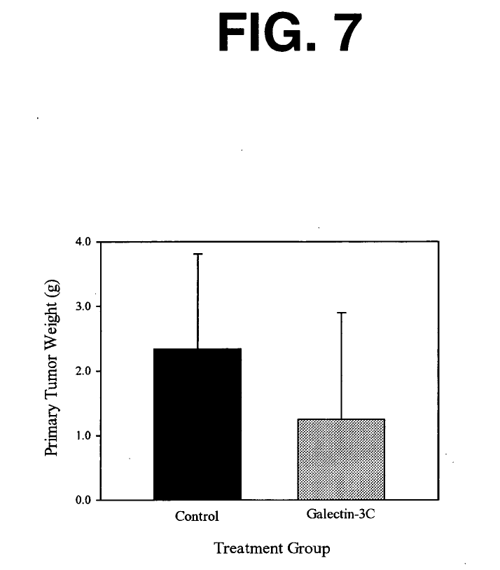

Upon autopsy, all visible primary tumors were removed and weighed using an electronic balance. Table 3 shows a comparison of the mean tumor weights at autopsy of mice in each treatment group. The mean tumor weights of the vehicle-only control and the galectin-3C treated groups are shown graphically in FIG. 7. The mean tumor weight in the mice treated with N-terminally truncated galectin-3 was significantly less than that in the control mice (p=0.007), whereas the mean tumor volume in mice treated with galectin-3 was similar to that in the control mice (p=0.634).

TABLE 2
Mean tumor volumes at autopsy*

| | | Mean primary | |
|---|---|---|---|
| | | tumor volume | |
| Group | Number of Mice | (mm³) ± SD | P value** |
| Vehicle Control | 20 | 2368.4 ± 1732.7 | — |
| N-terminally truncated | 20 | 1149.2 ± 1679.7 | 0.003 |
| galectin-3 | | | |
| Galectin-3 | 5 | 2307.1 ± 1718.0 | 0.865 |

*Tumor volume was calculated by the formula W² × L/2, where W is the smallest dimension.
**All treated groups compared to vehicle control by the Mann-Whitney U test.

TABLE 3
Mean tumor weights at autopsy

| | | Mean primary | |
|---|---|---|---|
| | | tumor weight | |
| Group | Number of Mice | (g) ± SD | P value* |
| Vehicle Control | 20 | 2.34 ± 1.47 | — |
| N-terminally truncated | 20 | 1.25 ± 1.65 | 0.007 |
| galectin-3 | | | |
| Galectin-3 | 5 | 2.30 ± 1.44 | 0.634 |

*All treated groups compared to vehicle control by the Mann-Whitney U test.

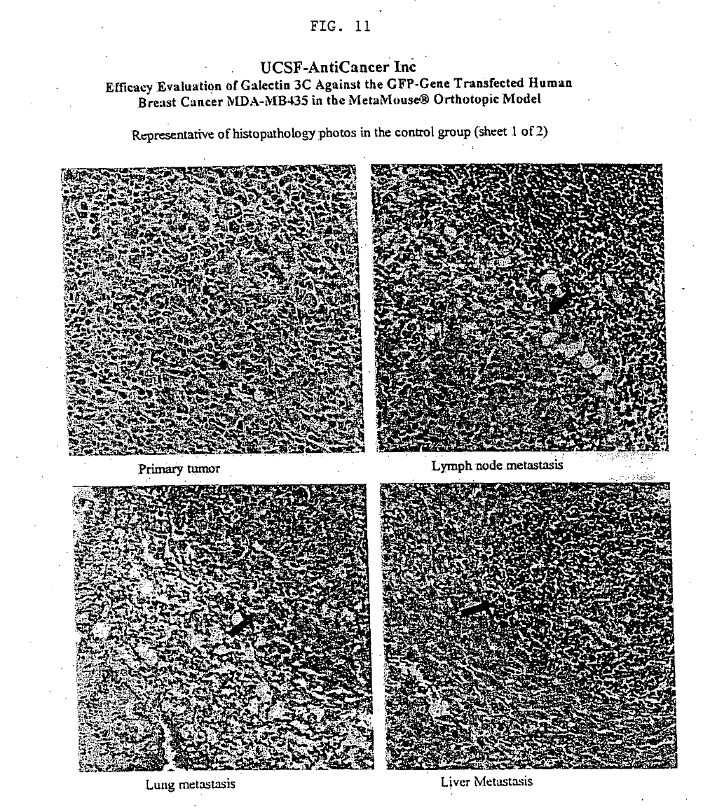

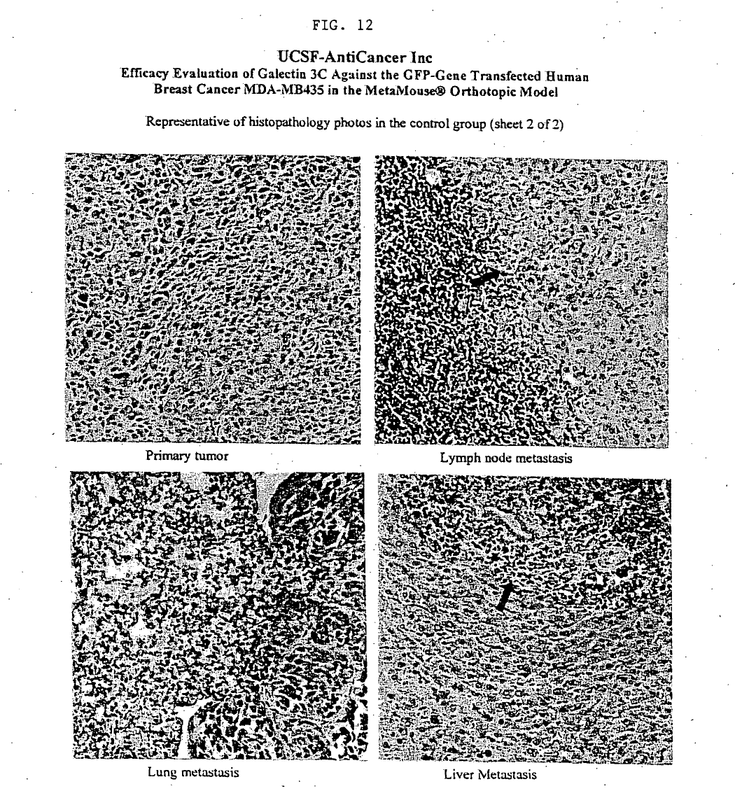

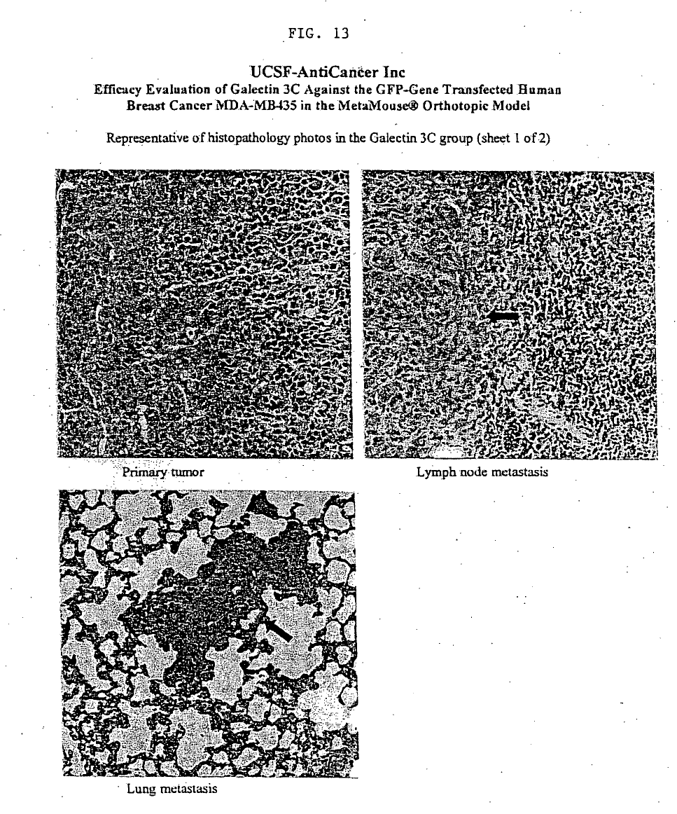

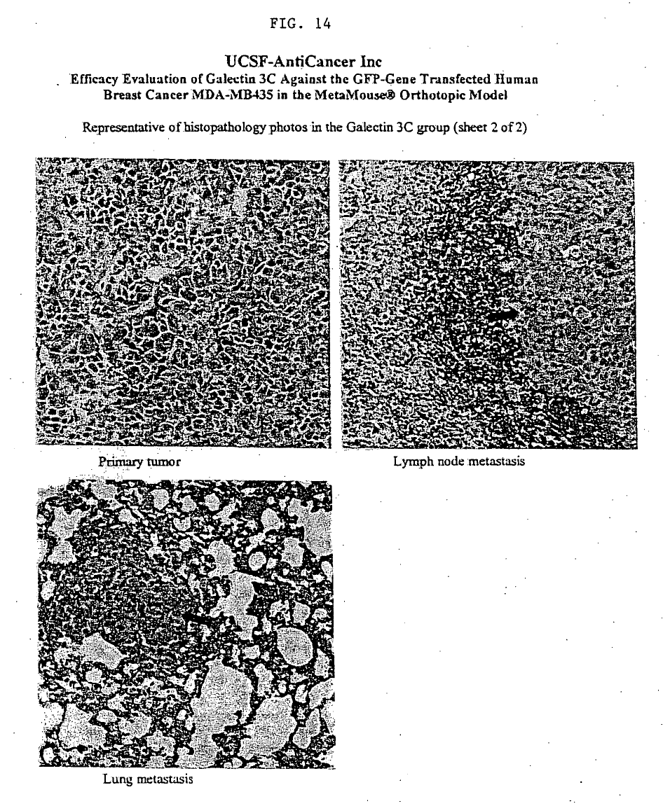

Assessment of metastasis: At autopsy, tissue samples from the auxiliary lymph node, the liver, and the lungs were collected and processed through standard procedures of hematoxylin and eosin staining for subsequent microscopic examination. Representative histopathology of the primary tumors in each group is shown in FIGS. 11-15. No significant pathological differences within primary tumors were noted among the groups.

Metastasis to the auxiliary lymph node, the lung, and the liver was assessed microscopically. Representative histopathology of the metastatic tumors in each group is shown in FIGS. 11-15. Table 4 shows the results of this assessment. Statistical analyses were carried out using the Chi-square/Fisher exact test. Eleven out of twenty mice had auxiliary lymph node metastasis in the control group whereas only four of the twenty mice developed auxiliary lymph node metastasis in the N-terminally truncated galectin-3 group by the end of the study (p<0.05). The incidence of metastases in the liver and the lung between the N-terminally truncated galectin-3 group and the control group was not different. Treatment with galectin-3 did not demonstrate a significant difference in metastatic incidence from the control. Tables 5 and 6 show the metastatic data as a function of tumor volumes categorized as small, medium, and large. A comparison of the two Tables reveals that five out of the eight small tumors in the control group had associated metastases while 0 of the 15 small tumors in the N-terminally truncated galectin-3 group had associated metastases. This shows that a reduction in tumor volume by N-terminally truncated galectin-3 results in a decreased incidence of metastases.

TABLE 4
Incidence of metastases in lymph node, lung and liver

| | Number | Lymph | P | | P | | P |
|---|---|---|---|---|---|---|---|
| Group | of Mice | Node | value* | Lung | value | Liver | value |
| Vehicle | 20 | 11/20 | — | 3/20 | — | 2/20 | — |
| Control | | | | | | | |
| Truncated | 20 | 4/20 | 0.022 | 4/20 | 1.0 | 0/20 | 0.487 |
| galectin-3 | | | | | | | |
| Galectin-3 | 5 | 1/5 | 0.322 | 1/5 | 1.0 | 0/5 | 1.0 |

*All treated groups compared to vehicle control by the Chi-square/Fisher exact test.

TABLE 5
Number of metastases in control group as a function of tumor volume

| Metastatic | Small tumors | Medium tumors | Large tumors |
|---|---|---|---|
| assessment | (0-1500 mm³) | (1501-2999 mm³) | (>2999 mm³) |
| metastases | 5 (LN only) | 2 (LN only) | 4* |
| no metastases | 3 | 5 | 1 |

*All 4 mice had lymph node metastases; 2 of those had liver and lung metastases, and 1 of those had lung metastases

TABLE 6
Number of metastases in N-terminally truncated galectin-3 group
as a function of tumor volume

| Metastatic | Small tumors | Medium tumors | Large tumors |
|---|---|---|---|
| assessment | (0-1500 mm³) | (1501-2999 mm³) | (>2999 mm³) |
| metastases | 0 | 2 (2 lung; 0 liver)* | 2 (2 lung; 0 liver)* |
| no | 15 | 1 | 0 |
| metastases | | | |

*Both mice had lymph node and lung but not liver metastases

In treated animals the number of primary tumors that metastasized was less than controls by a factor of 2.75 (p=0.022), and the mean primary tumor weights of the treated were approximately 2-fold less by the end of the 90-day study (p=0.007).

Statistical methods used in efficacy evaluation (120): Animal body weight comparisons were performed by Student's t-test. The primary tumor volume and weight were evaluated by the Mann-Whitney U test. The incidence of metastasis in the lymph node, lung, and the liver were evaluated by the Chi-square/Fisher-exact test. All tests were two-sided with α=0.05.

In addition to the statistical data analyses of the efficacy data described above, an analysis of tumor volume growth over time was performed using a repeated measures statistical model for log tumor volume, with cage in random statement. Specifically, this was a SAS Proc Mixed analysis to fit a linear mixed model with random effects for cage and mouse slope and intercept. The fitted model was quadratic in treatment day. The model was applied to the vehicle control and N-terminally truncated galectin-3 treatment data for tumor volume that showed a significant difference at day 90 when tested with the Mann-Whitney U test. The model allowed us to sensitize the analysis by factoring out non-treatment parameters such as cage and mouse variation, tumor volume trajectory differences within a group, and residual unexplained variation.

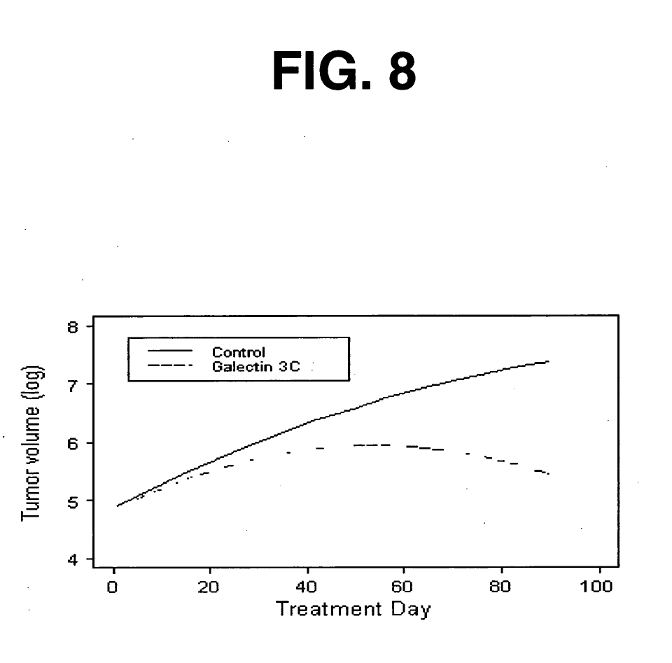

As shown in FIG. 8, the mean tumor volume in the group of mice treated with N-terminally truncated galectin-3 is statistically significantly less than that in the vehicle control group during more than 50% of the treatment period. At day 45 of treatment, which is labeled as TxDayCenter 0 on the x-axis, the slope of the tumor volume curve for the N-terminally truncated galectin-3 group is significantly different than that of the vehicle control group (p=0.045; SAS type 3 F test). Following day 45, treatment with N-terminally truncated galectin-3 resulted in increased differences between the slopes and trajectories of the two lines representing the mean tumor volumes of the two groups of mice. This model strongly supports the conclusion that N-terminally truncated galectin-3 was efficacious in reducing the number of metastases and as well as tumor growth over time in the orthotopic implantation mouse model of breast cancer.

The maximum tolerated dose of N-terminally truncated galectin-3 in nude mice at five days was determined to be greater than 125 mg/kg without overt adverse effects. This suggested that the safety threshold for the use of N-terminally truncated galectin-3 in vivo is high. The pharmacokinetic analysis of the intramuscular administration of ³⁵S-labeled N-terminally truncated galectin-3 into nude mice indicated an elimination half-life of N-terminally truncated galectin-3 of 3.01 hours in serum and 4.26 hours in the cellular fraction of the blood. The effort to produce antibody to the carbohydrate recognition domain of human galectin-3 in chickens was not successful very likely due to the homology in this domain of galectin-3 among various species. Organ biodistribution analyses showed that N-terminally truncated galectin-3 localized to the liver, kidney, and spleen but not to the heart or lungs.

Based on the pharmacokinetic and maximum tolerated dose data and the amount of N-terminally truncated galectin-3 that was produced, mice were treated twice daily with N-terminally truncated galectin-3 for 90 days and the results were compared with the treatment efficacy of mice treated with vehicle only. At the end of the treatment regime, it was found that the mean tumor volume and mean tumor weight were statistically significantly less in the group of mice treated with N-terminally truncated galectin-3 compared with the group of mice treated with the vehicle control. Repeated measures of the statistical model for tumor growth over time showed that the mean tumor volume was statistically significantly less by day 45 of the 90 day treatment regime in the group of mice treated with N-terminally truncated galectin-3 compared with the group of mice treated with the vehicle control. In addition, there were statistically significantly fewer numbers of mice that had lymph node metastases in the group of mice treated with N-terminally truncated galectin-3 compared with the vehicle control group of mice.

The hypothesis that was tested was that therapy with recombinant N-terminally truncated galectin-3 is efficacious for inhibition of tumor invasion and metastasis in breast cancer. Taken together, the data generated in this study strongly support this hypothesis. It was found that N-terminally truncated galectin-3 was efficacious in reducing breast cancer metastases in the orthotopic implantation mouse model of breast cancer. The average volume of the tumors in the treated group was less than that of the vehicle-only control group beginning on day 14 until the end of the study, reaching statistical significance by day 63 using the T test to compare the groups. The mean tumor weight of the galectin-3C treated group upon necropsy was less than the control group (p=0.007) by a factor of almost two-fold. Only two animals in the treated group had large tumors of a size greater than 2,999 mm³, whereas 5 animals in the control group had tumors of this size. These data support the conclusion that treatment of breast tumor-bearing mice with galectin-3C significantly reduced the progression of tumor growth.

The result of the SAS Proc Mixed analysis also strongly supports the conclusion that galectin-3C was efficacious in reducing the number of metastases as well as actually shrinking tumors and inhibiting tumor growth over time. Further evidence is gained by the fact that in the treated group in some individual mice tumor growth slowed and tumor volume regressed, and that there was no primary tumor observed in 3 treated animals (15%) upon necropsy at the end of the 90-day study.

In general, orthotopically transplanted tumors are more clinically relevant than subcutaneous xenograft models due to the physiological location of the tumor and the spontaneous hematogenous metastases (121). Treatment with galectin-3C reduced the percentage of animals with metastases from 55% (11/20) in the control group to 20% (4/20) in the treated group, a decrease of 2.75-fold. None of the animals in the treated group with small tumors less than 1500 mm³ in size (0/15) had metastases, whereas 62.5% (⅝) of the control group that had tumors this size had metastases. Only one animal in the treated group had a tumor larger than 1500 mm³ that had not metastasized compared to 6 animals in the control group that had tumors of this size that had no metastases. Apparently animals in the treated group that had metastatic tumors were not responding to galectin-3C as their tumors were larger as well.

Importantly, it was found that N-terminally truncated galectin-3 also reduced tumor volume and tumor weight in the primary tumors. The mechanism of action of extracellular N-terminally truncated galectin-3 was likely to be at least partly due to: 1) decreased homotypic aggregation and adhesion of breast cancer cells to extracellular matrices and endothelial cells; 2) decreased chemotaxis (galectin-3 itself is a chemotactic factor) (57); or 3) anoikis (apoptosis induced by loss of cell anchorage) [Kim, 1999 #955.

The development of a system for detection of N-terminally truncated galectin-3 in nude mice using ³⁵S-labeled N-terminally truncated galectin-3 allowed the accurate determination of the in vivo pharmacokinetics of intramuscularly injected N-terminally truncated galectin-3. The fact that N-terminally truncated galectin-3 exhibited no detectable toxicity and reduced tumor volume, tumor weight, and incidence of metastases in a nude mouse model of breast cancer strongly supports the use of N-terminally truncated galectin-3 as a therapeutic compound for the treatment of breast cancer and other types of cancer. The results indicate that galectin-3C has minimal overt toxicity and that it has utility in treatment of breast cancer by shrinking tumors and reducing tumor growth over time, and by inhibiting metastasis.

Alternatively, an antibody to the specific complex carbohydrate ligands of galectin-3 has a longer half-life than galectin-3C and inhibits galectin-3 binding and multimerization as does the N-terminally truncated galectin-3 protein. Such an antibody also increases the efficacy obtainable. Thus, the rationale for either a sustained release form of the protein or an antibody to galectin-3 carbohydrate ligands is the same, to increase the efficacy, cost-effectiveness, and convenience of the therapy.

Breast cancer is the most frequently diagnosed cancer in women in the U.S., other than non-melanoma skin cancer that has a much lower mortality rate, according to figures from the American Cancer Society. Despite the available treatments breast cancer ranks second for women as the cause of death from cancer, and the lifetime risk of breast cancer for women living in the U.S. is now 1 in 8. Among those factors predictive of risk of recurrence of breast cancer are age, tumor size, hormone receptor status, and histologic type and grade, metastatic carcinoma in axillary lymph nodes is more important than any other currently available (122,123). Metastatic breast cancer has a poor prognosis especially if the tumor is hormone-independent, and is the primary cause of mortality in individuals already affected. Fewer than 10% of patients with metastatic breast cancer survive 5 years or more (124). There is evidence that galectin-3 plays a role in other types of cancer including thyroid, colon, and prostate, hence, these types of cancer are also susceptible to treatment with galectin-3C.

### Example 2

As background for the following example, the first PEGylated protein that was approved for use in the United States by the Food and Drug Administration was PEG-adenosine deaminase (PEG-ADA), for patients with severe combined immunodeficiency disease. Second to be approved was PEGylated asparaginase, used for the treatment of acute lymphoblastic leukemia. Also, the PEGylated version of interferon-alpha has been approved for human use (125).

Most proteins are cleared from the circulation by the reticuloendothelial system (RES), kidney, spleen, or liver. Clearance depends on the size, charge, glycosylation, and cellular receptors of the protein. Metabolism by proteases and peptidases also can lead to loss of biological activity and degradation. Modification of proteins with PEG can extend their half-life from 3 to 486-fold (126). The extension of half-life can be partly due to increasing the molecular weight of the modified protein until it is large to make the cut-off for glomerular filtration, that is somewhere between molecular mass 67 and 68 kDa (126), thus, necessitating addition of a 40 or 50 kDa PEG to a small protein. Larger proteins are often cleared by the RES or specific cellular receptors. This clearance also can be reduced by PEGylation. Modification with PEG also can reduce proteolytic degradation.

A drawback of PEGylation can be reduced biological activity and this has been found to occur with many proteins (101,127). Derivatization of the active site of an enzyme or a residue in a biologically important binding domain of a protein can lead to inactivation. Site-specific modification a one or a few residues of the protein can be used to avoid this problem. Steric hindrance obtained with a bulky PEG derivative can be used to decrease reactivity at some sites. Performing the derivatization reaction in the presence of ligand or substrate can decrease reactivity of some amino acid residues. Lowered stoichiometry of PEG derivatives can also be exploited to alter product ratios. The pH of the reaction can be altered in derivatization with amino reactive PEGs to limit their reactivity.

One method for forming derivative proteins is to use PEGs that react with nitrogens on lysine or terminal amines to form stable amide linkages, such as mPEG-succinimidyl propionate or butanoate. With reagents of this type, the use of a basic pH in the reaction can result in a mixture of products with PEGylation at all lysine residues where lower pH can limit the derivatization to a single residue. Site-specific modification of a single His residue of interferon-alpha-2B was achieved using a reaction with succinimidyl carbonyl PEG at pH 5.4 (128). Using N-hydroxysuccinimidyl carboxymethyl at pH 5, PEG enabled site-specific modification of the N-terminus of human granulocyte stimulating factor (129).

One method for derivatizing proteins with PEG is to use thiol-reactive PEGs that forms covalent bonds with cysteine residues, such as mPEG-maleimide (Shearwater Corporation, Huntsville, Ala.) (101,103,105). The endogenous cysteine (Cys-173 of human galectin-3) of N-terminally truncated galectin-3 can be used as a site for covalent modification with PEG. Alternatively, using genetic engineering, one or more additional cysteines can be added to either the C- or the N-terminal end of the N-terminally truncated galectin-3 protein. The cysteines can be used as sites to react with the thiol-reactive PEGS. The N-terminally truncated galectin-3, with and without additional cysteines added by genetic engineering, can be produced by cloning methods.

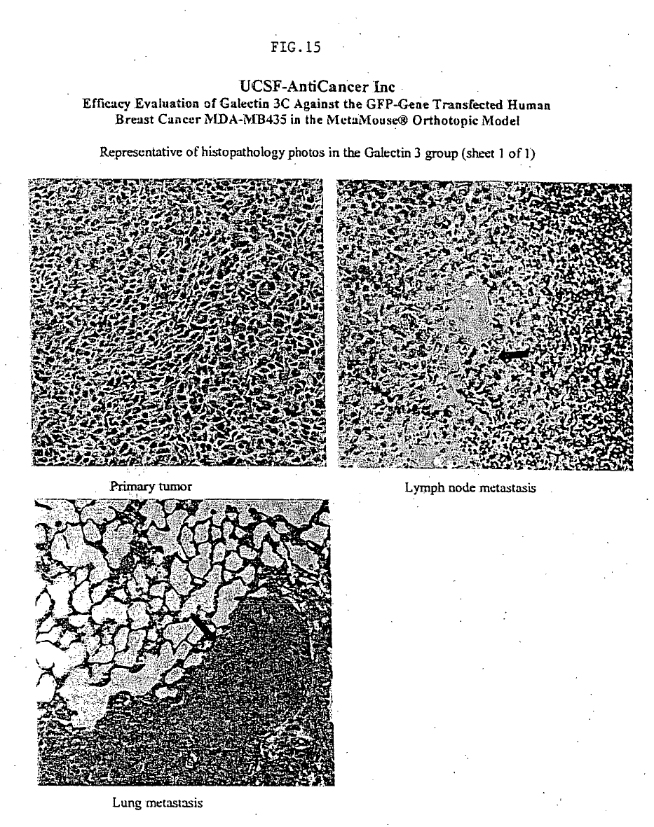

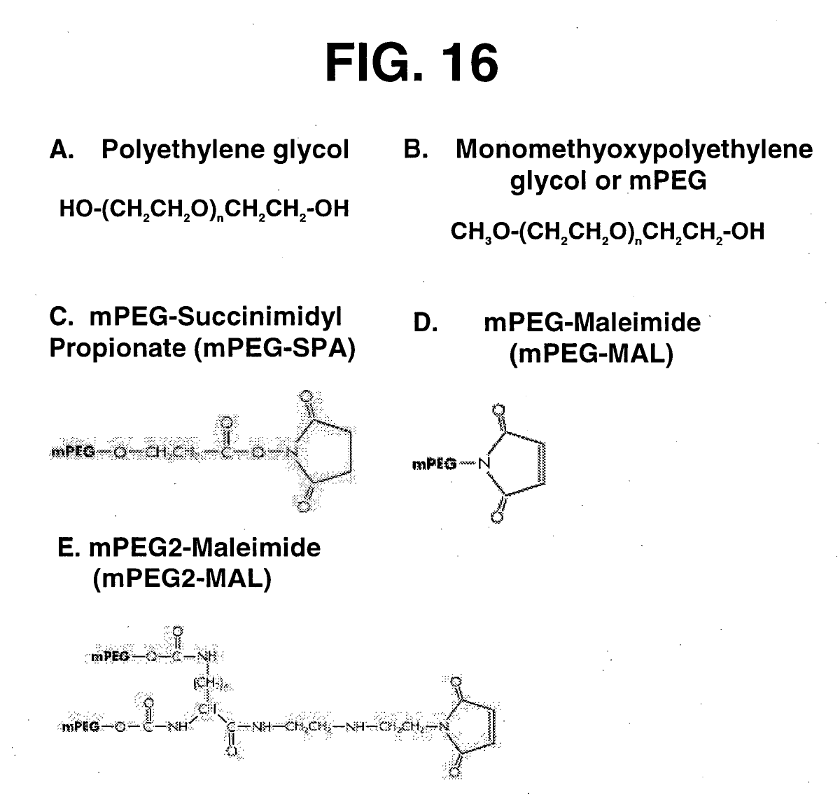

The relative short half-life of the intramuscularly injected N-terminally truncated galectin-3, the lack of acute toxicity of higher doses, and the efficacy demonstrated by the 4 mg/kg dose given twice daily provides clear rationale for formulation of a sustained release form of the protein, for example using a PEG derivatization, to increase the half-life. The sustained release form enables higher levels of the protein to be sustained in the body and thereby obtains greater efficacy with more convenience and less expense. One method to achieve site-specific derivatization is to use sulfhydryl reactive PEG derivatives as shown in FIG. 15 and 16. The N-terminally truncated galectin-3, with and without additional cysteines added by genetic engineering, can be produced by known cloning methods.

A plasmid containing the complete galectin-3 coding sequence is used as a template in a PCR reaction using primers designed to amplify the desired fragment.

Forward primer:
5′ GACGACGACAAGtgcGGCGCCCCTGCTGGG 3′
Reverse primer:
5′ GAGGAGAAGCCCGGTTTATATCATGGTATA 3′

The reverse primer defines the carboxy-terminal protein sequence and in this example does not differ from that required to produce recombinant human galectin-3. The non-underlined portion of the forward primer 5′-GACGACGACAAGtgcggcgcccctgctgggccactg-3′ introduces a cysteine immediately upstream of the N-truncated sequence providing an additional site on which PEG can be attached (starting first with a cysteine then continuing with the rest of the amino acid sequence glycine, alanine, proline, alanine, etc.). The underlined sequences are added as tails that can be used to fuse the PCR product with pET32 Ek/LIC plasmid using the Ek/LIC ligation protocol (Novagen, Madison, Wis.). This plasmid produces a fusion protein with a variety of unique binding qualities and endoprotease sites allowing for high yields and purity of the recombinant protein. More than one cysteine can be introduced to the construct by simply including more cysteine codons (either tgt or tgc) to create a version of N-truncated galectin-3 with multiple cysteines. One or more cysteines can be added to either the C or N-terminus for derivatization with PEG using this method. The site of PEGylation can affect the carbohydrate binding or the biological localization of the truncated galectin-3 molecules and the bioactivity of the molecule can vary accordingly. The relative affinity for carbohydrate ligands and the localization of derivatized truncated galectin-3 molecules can be determined readily by those with skill in the art using standard methodologies.

The E. coli BL21 (DE3) bacteria is transformed with this construct and can be used for protein production. Expression is under the control of bacteriophage T7 transcription and is induced by providing a source of T7 RNA polymerase, such as infection with a phage that carries the T7 RNA polymerase gene or moving the plasmid into a cell containing an expression host containing a copy of the T7 RNA polymerase gene.

Cloning.

The PCR products are purified using spin columns, and then ligated into pET32 following the vendor's instructions. The ligated product is transformed into competent Novablue bacteria and clones are selected based on blue-white selection. DNA was extracted from picked clones and analyzed by gel electrophoresis, PCR, and sequencing. The plasmid is sequenced using the same forward and reverse primers as PCR using an ABI 3700 machine. This DNA is used to transform BL21 strain of E. coli. Isolated colonies were picked, grown in culture, and glycerol stocks are stored for future use.

Growing Bacteria.

Luria-Bertani (LB) broth with ampicillin (50 mg/l) was inoculated with 20 μl/L of glycerol frozen stock of transformed E. coli. The culture is shaken at 37° C. overnight. Then, the inducer, isopropyl-β-thiogalactopryranoside (IPTG), is added (100 mg/l) and the culture is incubated at 37° C. for an additional four hours. The cells are centrifuged to pellet them, and the supernatant discarded, and the pellet is stored at −20° C.

Lysis of Bacteria.

To improve reproducibility and yield of protein a commercially available cell lysis reagent is used. First the cell pellet is thawed at room temperature, and then B-Per Bacterial Protein Extraction Reagent (Pierce Endogen, Rockford, Ill.) was added (25 ml/500 ml) with 1 mM phenylmethanesulfonyl fluoride (PMSF; Sigma, St. Louis, Mo.), a serine protease inhibitor. The cells are resuspended by vortexing until there were no clumps, and then the vials are shaken at room temperature for 10 minutes, and spun at 27,000 g for 10 minutes. The supernatant is the cell lysate containing the soluble proteins and was stored at 4° C.

Affinity Chromatography.

The protein product is purified by affinity chromatography on a lactosyl-Sepharose column. Bacterial lysates are loaded and the column washed with 5 volumes of phosphate-buffered saline (PBS) with 4-mM 2-mercaptoethanol, 2 mM EDTA, and 0.5 mM PMSF. The galectin-3 proteins are eluted with 10 volumes of PBS with 150-mM lactose and 0.5 mM PMSF. The amount of purified protein is determined using the BioRad Protein Assay (Hercules, Calif.) according to the vendor's instructions. Enterokinase Digestion.

The N-terminal fusion tags are cleaved by digestion with porcine enterokinase enzyme (Sigma) at 0.7 U per mg of galectin-3 protein. The digestion is performed in 20 mM TRIS at pH 5.6 at room temperature for 24-hours.

Derivatization of Cys-(Δ1-107) galectin-3 with mPEG2-MAL

The (Δ1-107) protein with an additional Cys residue on the N-terminus is derivatized with the mPEG2-maleimide (mPEG2-MAL) molecule (Shearwater Corporation) that has a molecular weight of 40,000, and reacts specifically with thiol groups. A slight molar excess of mPEG2-MAL is added to N-terminally truncated galectin-3 ((Δ1-107) galectin-3 with an additional single cysteine at the N-terminus) with a 10-fold molar excess of lactose at pH 7.5 in PBS at room temperature for two hours. After dialysis to remove the lactose, the PEGylated N-terminally truncated protein is purified on affinity chromatography using lactosyl-Sepharose.

### Example 3

Polymeric microspheres have been used in formulations of proteins to achieve sustained delivery, and have been approved as part of several different products for humans. Biodegradable poly(lactic-co-glycolic acid) (PLGA) has been widely used as a material for microencapsulation to attain sustained release (130-132). The microspheres are produced by a number of techniques, including freeze-drying or atomizing with gas anti-solvent CO₂ precipitation (130-133). The resultant nanoparticles are analyzed by scanning electron microscopy (134).

Encapsulation of (Δ1-107) galectin-3 with poly(lactic-co-glycolic) Acid microspheres.

A modification of the procedure of Lam et al. that was used to achieve a product that produced controlled release of nerve growth factor over a period of 14 days was employed (131). Purified (Δ1-107) galectin-3 is formulated at 5 mg/ml in two buffer systems and then lyophilized. The buffer systems can be a 5 mM histidine at pH 5.5 or a 4 mM sodium bicarbonate at pH 7.4 with various concentrations of zinc acetate. Lactose at 5 mg/ml is also added to the buffers. Next, PLGA (1.7 g; 50:50 lactide/glycolide, 12 kDa, RG502H; Boehringer Ingelheim Chemicals (Winchester, Va.) is added.

The stability of the lyophilized protein is determined by adding 5 mg of protein from each of the protein formulations to 0.5 ml of ethyl acetate. The samples are homogenized at 8,000 rpm for one minute and then the protein is recovered by dilution into 50-fold excess of 5 mM histidine, pH 5.5. Ultraviolet spectroscopy (280 nm) and affinity chromatography are used to assess the amount and the stability of the protein. The lyophilized formulations containing the greatest amount of recoverable protein are chosen for freeze-drying for encapsulation by PLGA microspheres.

PLGA (1.7 g) is stirred into 10 ml of ethyl acetate at 5° C. and then solid (Δ1-107) galectin-3 (10% w/w), and zinc carbonate (0-6% w/w), a release modifier, are added. The suspension is homogenized at 8,000 rpm for one minute, and then poured into a 10-mi glass syringe and infused into an ultrasonic spray nozzle using a pump. The frequency of the spray is 120 kHz into a 2-L round-bottomed flask immersed in a liquid nitrogen bath and containing 300 ml of ethanol frozen underneath liquid nitrogen. The frozen microspheres form upon spraying and then settle on the top of the solid ethanol. The flask is stored at −70° C. where the ethanol melted and the ethyl acetate is extracted by the ethanol. After 24 hours, 300 ml of cold ethanol is added, and then after another two days a 20-micron filter is used to removed the ethanol from the microsphere suspension for drying. The microspheres are dried using nitrogen gas at 5° C., and then sieved with a 125-micron stainless steel mesh.

In vivo release is determined by resuspending the microspheres in PBS and incubating them at 37° C. in the reservoir of a centrifugal filtration device. After release, the (Δ1-107) galectin-3 is obtained by centrifugation, and additional PBS release buffer is added. The integrity of the protein is determined by confirmation of the carbohydrate binding capacity to lactosyl-Sepharose.

### Example 4

The carbohydrate-binding sites that are recognized by galectin-3 are widely expressed in tissues and on cells in various species. Antibodies that have high affinity for the complex carbohydrates recognized by galectin-3 specifically block the binding of galectin-3 to these sites in the body. Thus, such antibodies have the ability to block metastasis by blocking the multimerization and protein-protein interactions of galectin-3. The effects of the antibodies are similar to the effects obtained with N-terminally truncated galectin-3C. In other words, the antibodies reduced metastasis and tumorigencity. However, antibodies that have affinity and specificity to molecules that are commonly expressed in the body are more difficult to produce. A successful alternate approach to use of the carbohydrate ligands as antigens themselves to induce the formation of specific, high-affinity antibodies is to use phage-display libraries of random peptide sequences to select for peptide mimics of the carbohydrate structures that are then conjugated to highly immunogenic proteins (135-138). Then the peptide conjugates are used to generate antibodies.

Initially, the peptides are expressed as a library in the coat proteins of bacteriophage. Thus, binding to the virion can be used to assess affinity for the peptide expressed. The DNA encoding the peptide of interest can be sequenced readily and, thus, the structure of the peptide determined. Clones of interest are chosen by a process of enrichment called bio-panning. The protein is immobilized on a plate or on beads and incubated with the phage-display library. Unbound phage are washed away, and the bound phage are eluted. The eluted phage are amplified by being taken through the cycle of panning several times to enrich for sequences that bind. Individual clones are characterized by DNA sequencing. Using this approach Ishikawa and Taki (137) derived the sequence of peptides from phage clones that mimicked nLc₄Cer, a ligand of galectin-3 that has a nonreducing terminal N-acetyllactosamine unit linked betal-3 to lactose (paraglobosyl; lacto-N-neotetraose).

Bio-panning Peptide Phage Display Library for Peptide Mimetics of Carbohydrate Ligands of Galectin-3.

A library of random peptides (7 or 12-mers) is displayed when fused to a coat protein of M13 Phage. The libraries are available from New England BioLabs (Beverly, Mass.) and are screened for binding to N-terminally truncated galectin-3 following the instructions of the vendor. The library of 7-mers (Ph.D.-7) contains 2.0×10⁹ independent clones that represent most of the 20 of 1.28×10⁹ possibilities, and the library of 12-mers (Ph.D.-12) contains a similar number of independent clones, 1.9×10⁹, but this is not exemplary of the possible number of 12-mer sequences (20¹² or 4.1×10¹⁵). The protein can be immobilized to a carboxymethylated dextran-coated biosensor cuvette (Affinity Sensors, Cambridge, U.K.). The phage (2×10¹¹) is reacted with the immobilized protein for 60 minutes at 25° C. in PBS with 0.5% (v/v) Tween-20, and then washed to remove unbound phage. The bound phage is disassociated from the protein by treatment with acidic TRIS-glycine buffer and then neutralized with 1 M TRIS. Phage is amplified by transfection of E. coli strain E 3257 that is supplied by New England Biolabs and titered. The phage from this bio-panning is titered and bio-panned a third and a fourth time. Then the phage is plated without amplification on a lawn of E. coli, and individual phage (25-30) are picked from the lawn. The DNA is sequenced and the translated peptides for each clone are then determined. Typically, redundant and consensus peptides are derived. The peptides expressed by the greatest number of clones are synthesized.

Detection of Binding of Galectin-3 to Phage by ELISA.

Microtiter wells are coated overnight at 4° C. with about 10¹¹ CsCl-purified virions, 1 mg of immune-radio assay (I.R.A.) grade bovine serum albumin, or buffer alone. After washing with TRIS-buffered saline with Tween-20, various concentrations of galectin-3 protein is added and the plates incubated for 12 hours at 4° C. After washing three times, monoclonal antibody to galectin-3 (139) conjugated to FITC is added, and the plate incubated for another 1 hour at 37° C. and washed again. An ELISA plate reader equipped with fluorescence detection is used to determine the relative degree of binding to the virions. The ability of known carbohydrate ligands of galectin-3 to inhibit the binding can be determined.

Production of Humanized Monoclonal Antibody to Galectin-3-binding Peptides.

The peptides or consensus peptides with the greatest affinity for galectin-3 can be conjugated to diphtheria toxoid or keyhole limpet hemocyanin and the conjugate can be used to immunize mice or other species. Monoclonal antibodies are produced using methodologies that are standard to those who are skilled in the art (reviewed in (140)). Isolation of B-cell clones expressing antibody molecules of a desired specificity and affinity are modulated by the immunogenicity of the antigen used for immunization. Size, complexity, and foreignness to the host all contribute to the immunogenicity of a molecule. In general, immunogens must have a molecular mass greater than 10 kDa. Globular proteins tend to be more immunogenic than carbohydrates, lipids, or nucleic acids.

Most peptides are not immunogenic due to their small size so they must be conjugated to carrier proteins such as bovine serum albumin or keyhole limpet hemocyanin. A variety of cross-linking reagents are commercially available for this purpose. The use of peptide conjugates as immunogens results in the expansion of peptide-specific B-cell clones and clones specific for the conjugated or fused protein.

Test bleeds are analyzed before trying to generate the desired monoclonal antibodies because a high titer, specific response of the appropriate immunoglobulin isotype indicates clonal expansion. The immunogenicity of antigen can be enhanced by conjugation to immunogenic carrier molecules, emulsification in adjuvant, or both. Soluble proteins are emulsified in Freunds adjuvant or RIBI Adjuvant System. These adjuvants slowly release antigen over time and induce inflammatory responses. Conjugating soluble antigens to agarose beads or other particulates can stimulate phagocytosis and presentation of antigenic peptides to T-lymphocytes.

The sera from the immunized animal is used to isolate antibody-forming B cells that are fused with a mutant myeloma cell line that has been mutagenized and selected to be defective in hypoxanthine-guanine phosphoribosyltransferase (HGPRT) or thymidine kinase (TK) so that it does not grow in HAT (cell medium containing hypoxanthine, aminopterin, and thymidine). The myeloma cells are deficient in an enzyme required for the salvage pathway of nucleotide synthesis and die in HAT-containing medium. The aminopterin blocks normal nucleotide synthesis and the enzyme deficiency blocks utilization of hypoxanthine or thymidine in the salvage pathway. When the normal antibody-producing cells from the sera fuse to the mutagenized myeloma cells, the hybrids are able to synthesize DNA and grow in HAT medium. The fused cells are monoclonal antibody producing hybridomas.

The fused hybridomas are selected in vitro by growth in HAT medium and then “cloned” so that each well of a microtiter plate contains the progeny of one cell. The supernatants of the cells are screened for the presence of antibody with the desired specificity for peptide mimic and for the complex carbohydrate ligands of galectin-3. Screening can be done using an enzyme-linked immunosorbent assay (ELISA), whole cell ELISA, immunoprecipitation, or immunoblot. Further purification of a hybridoma from a positive well is performed by cloning in soft agar or limiting dilution. Those clones that produce antibody with the desired affinity are expanded for freezing and generation of stock solutions of monoclonal antibody. Monoclonal antibodies then are generated by tissue culture or ascites production, usually in mice. Antibody concentrations in tissue culture supernatants are usually in the range of 10-20 μg/ml, but can be as high as 100 μg/ml. Larger amounts of monoclonal antibody can be generated as ascites fluid in a peritoneal cavity of an animal that is MHC compatible with the myeloma fusion partner and spleen cell donor. As much as 10 mg/ml of monoclonal antibody can be secreted into abdominal ascitic fluid (140).

To produce a therapeutic agent for humans based on a rodent monoclonal antibody, the structure must be humanized to reduce its immunogenicity and prevent the development of human anti-mouse antibodies (141-145). Dozens of rodent antibodies have been humanized, there are many humanized monoclonal antibodies in clinical trials, and now a few of these have been approved as drugs. The rodent complementary determining regions (CDRs) of the antibody are grafted into the human IgG framework. Actually producing the engineered monoclonal antibody using the techniques of molecular biology is relatively simple. The technique is well established but the specificity and affinity of the humanized antibody must be comparable to the original. If intact IgG is used, this is typically produced in mammalian cells. Antibody fragments such as F(ab′)2, Fab′, Fab, and scFV are useful as therapeutic agents (146). These are often expressed by bacterial cultures that have shorter expression times, higher yields, and lower costs than mammalian culture systems. Full length IgG has a longer half-life than antibody fragments, but PEGylation of antibody fragments can be used to increase their circulating half-life.

### Example 5

Specific examples of modifications of the N-terminally truncated galectin-3 sequence that could be made include the following conserved mutation substitutions of one or more amino acids occurring between position 201 and 230. The amino acid on the left is the original one and the amino acid on the right is the one that put in the amino acid sequence as a substitute.

| | | |
|---|---|---|
| Val-202 | → | Ala |
| Val-204 | → | Ala |
| Glu-205 | → | Asp |
| Asp-207 | → | Glu |
| His-208 | → | Arg |
| Phe-209 | → | Leu |
| Val-211 | → | Ala |
| Ala-212 | → | Val |
| Asp-215 | → | Glu |
| Ala-216 | → | Val |
| His-217 | → | Arg |
| Tyr-221 | → | Phe |
| His-223 | → | Arg |
| Val-225 | → | Ala |
| Glu-230 | → | Asp |

In vitro site-directed mutagenesis is a technique that can be used for carrying out a substitution at one or several sites specifically. Several approaches to this technique have been published, but these methods generally require single-stranded DNA (ssDNA) as the template and are labor intensive or technically difficult. Stratagene's (La Jolla, Calif.) QuikChange® XL site-directed mutagenesis kit (U.S. Pat. Nos. 5,789,166; 5,923,419; 6,391,548 and patents pending) allows site-specific mutation in virtually any double-stranded plasmid, thus eliminating the need for subcloning into M13-based bacteriophage vectors and for single-stranded DNA rescue.

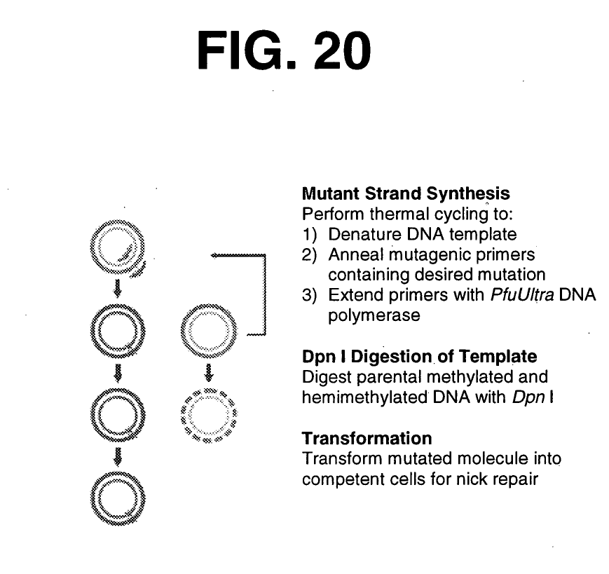

The QuikChange XL system is outlined in FIG. 20. It is used to make point mutations, switch amino acids, and delete or insert single or multiple amino acids. The QuikChange XL system requires no specialized vectors, unique restriction sites, multiple transformations, or in vitro methylation treatment steps. This three-step procedure generates mutants with greater than 80% efficiency. The protocol is simple and uses either miniprep plasmid DNA or cesium-chloride-purified DNA. Stratagene's QuikChange Multi System enables mutagenesis at multiple sites in a single round, using a single oligonucleotide per site. It also makes it easy to randomize key amino acids using oligos containing degenerate codons. A rapid three-step procedure introduces mutations at three different sites simultaneously in the 4-kb QuikChange Multi control plasmid with greater than 50% efficiency. The QuikChange XL method is performed using PfuUltra™ high-fidelity (HF) DNA polymerase (U.S. Pat. Nos. 6,183,997; 6,333,165; 6,379,553; 5,948,663; 5,866,395; 5,545,552; 6,444,428; 5,556,772 and pending) and a thermal temperature cycler for mutagenic primer-directed replication of both plasmid strands with the highlest fidelity. The basic procedure utilizes a supercoiled double stranded DNA (dsDNA) vector with an insert of interest and two synthetic oligonucleotide primers containing the desired mutation (see FIG. 20). The oligonucleotide primers, each complementary to opposite strands of the vector, are extended during temperature cycling by using PfuUltra HF DNA polymerase. Extension of the oligonucleotide primers generates a mutated plasmid containing staggered nicks. Following temperature cycling, the product is treated with Dpn I. The Dpn I endonuclease (target sequence: 5′-Gm6ATC-3′) is specific for methylated and hemimethylated DNA and is used to digest the parental DNA template and to select for mutation-containing synthesized DNA. (DNA isolated from almost all E. coli strains is dam methylated and therefore susceptible to Dpn I digestion.) The nicked vector DNA containing the desired mutations is then transformed into XL1-Blue supercompetent cells (Stratagene). Unwanted second-site errors are virtually eliminated and high mutation efficiencies are obtained using this method due to the high fidelity of the PfuUltra HF DNA polymerase, the use of a small amount of starting DNA template and the use of a low number of thermal cycles.

Amino acid substitutions are performed using a PCR-based site-directed mutagenesis kit. To make the substitution of Asp-207→Glu, the oligonucleotide primer used is the following sequence.

5′ ACT GTT GAA CCT GAA CAC TTC AAG GTT 3′

The complementary oligonucleotide primer is the following.

5′ AAC CTT GAA GTG TTC AGG TTC AAC ACT 3′\

The new N-terminally truncated galectin-3 construct can be produced by other cloning methods, and it is designated as SEQ ID NO. 4, and is as follows:

gap agplivpynl
plpggvvprm litilgtvkp nanrialdfq rgndvafhfn prfnennrrv ivcntkldnn
wgreerqsvf pfesgkpfki qvlvepehfk vavndahllq ynhrvkklne isklgisgdi
dltsasytmi

The protocol for preparation of this construct is as follows (see FIG. 20). Briefly, the mutant strand synthesis reaction is prepared as indicated in thin-walled tubes. The control reaction contains the following: 2.5 μl of Reaction Buffer (10×); 2 μl (10 ng) of pWhitescript 4.5-kb control template; 1.25 μl each of oligonucleotide control primers #1 and #2; 1 μl of dNTP mix; 3 μl of QuikSolution reagent; 35.5 μl ddH₂O to a final volume of 50 μl. The sample reaction contains 5μl of 10× reaction buffer; 10 ng of ds DNA template; 125 ng each of oligonucleotide primers #1 and #2; 1 μl of dNTP mix; 3 μl of QuikSolution reagent; 35.5 μl ddH₂O to a final volume of 50 μl. To each control and sample reaction is added 1 μl of PfuUltra HF DNA polymerase (2.5 U/μl). Cycle each reaction using the parameters outlined below. Overlay each reaction mixture with about 30 μl of mineral oil if the thermal cycleter does not have a hot top assembly.

TABLE 7

| Segment | Cycles | Temperature | Time |
|---|---|---|---|
| 1 | 1 | 95° C. | 1 minute |
| 2 | 18 | 95° C. | 50 seconds |
| | | 60° C. | 50 seconds |
| | | 68° C. | 1 minute/kb of |
| | | | plasmid |
| 3 | 1 | 68° C. | 7 minutes |

Then add 1 μl of Dpm 1 restriction enzyme (10 U/μl) below the mineral oil overlay (if used). Next, gently and thoroughly mix each reaction, spin down in a microcentrifuge for 1 minute, and immediately incubate at 37° C. for 1 hour to digest the parental supercoiled DNA. Lastly transfor 2 μl of the Dpn 1-treated DNA from each control and sample reaction in separate 45-μl aliquots of XL10-Gold ultracompetent cells.

The QuikChange ® Multi site-directed mutagenesis kit offers a rapid and reliable method for site-directed mutagenesis of plasmid DNA at up to five different sites simultaneously. A single mutagenic oligonucleotide is required to mutagenize each site, using a double-stranded DNA template and following a one-day, three-step procedure. Several approaches to this technique have been published, but these methods generally require the use of single-stranded DNA (ss-DNA) template, generally allow mutagenesis at only one site per round, and are labor intensive and technically difficult. Stratagene's original QuikChange site-directed mutagenesis kit eliminated the need for subcloning into M13-based bacteriophage vectors and for ss-DNA rescue, making site directed mutagenesis studies simple and reliable, allowing oligo-mediated introduction of site-specific mutations into virtually any double-stranded plasmid. The QuikChange Multi site-directed mutagenesis system is based on a novel technology that allows mutagenesis at multiple sites in a single round, using a single oligonucleotide per site. The QuikChange Multi site-directed mutagenesis system also makes it easy to randomize key amino acids using oligos containing degenerate codons. No specialized vectors or unique restriction sites are needed to use the kit—virtually any plasmid of up to 8 kb is a suitable template. Using one degenerate codon-containing primer per site, the system is useful for creating mutant collections containing all possible amino acid side chains at one site as well as combinations of different amino acids at multiple sites. A collection of mutants can be created in a single QuikChange Multi kit reaction and then assayed for mutants with improved activity using any appropriate functional screen for your gene. The QuikChange Multi kit overcomes issues associated with mutagenesis methods that employ mutagenic primers corresponding to both strands of DNA, where representation of mutants in the collection can be limited by the preferential binding of complementary strands of mutagenic primers to each other.

To make substitutions of two amino acid residues simultaneously using the QuikChange Multi kit the following two primers are used together. To make the substitution of Val-225→Ala the oligonucleotide primer used is the following sequence.

5′ TAC AAT CAT CGG GCT AAA AAA CTC AAT 3′

To make the substitution of Asp-207→Glu the oligonucleotide primer used is the following sequence.

5′ ACT GTT GAA CCT GAA CAC TTC AAG GTT 3′

The new (Δ107) N-terminally truncated galectin-3 construct with two substitutions can be produced by other cloning methods as well and is designated as SEQ ID NO. 5, and is as follows:

gap agplivpynl
plpggvvprm litilgtvkp nanrialdfq rgndvafhfn prfnennrrv ivcntkldnn
wgreerqsvf pfesgkpfki qvlvepehfk vavndahllq ynhrakklne isklgisgdi
dltsasytmi

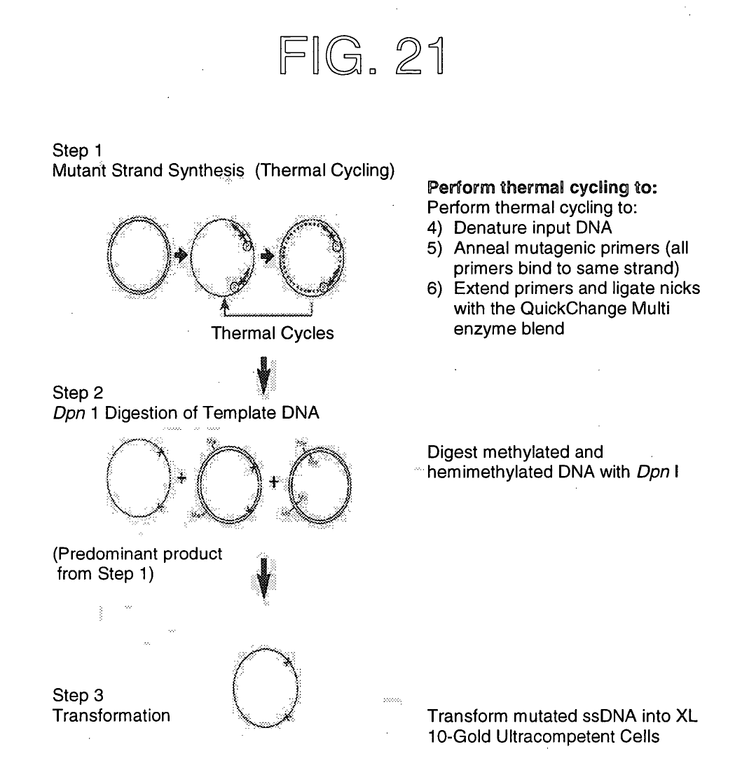

The three-step mutagenesis method is outlined in FIG. 21. Step 1 uses a thermal cycling procedure to achieve multiple rounds of mutant strand synthesis. Components of the thermal cycling reaction include a supercoiled double-stranded DNA template, two or more synthetic phosphorylated oligonucleotide primers containing the desired mutations, and the kit-provided enzyme blend featuring Pfu Turbo ® DNA polymerase. First the mutagenic primers are annealed to denatured template DNA. (Note that all oligonucleotides are designed to bind the same strand of the template DNA.) Pfu Turbo DNA polymerase then extends the mutagenic primers with high fidelity and without primer displacement, generating ds-DNA molecules with one strand bearing multiple mutations and containing nicks. The nicks are sealed by components in the enzyme blend. In Step 2 of the procedure, the thermal cycling reaction products are treated with the restriction endonuclease Dpn I. The Dpn I endonuclease (target sequence: 5′-Gm 6 ATC-3′) is specific for methylated and hemimethylated DNA 7 and is used to digest the parental DNA template. DNA isolated from almost all Escherichia coli strains is dam methylated and therefore susceptible to digestion. In Step 3, the reaction mixture, enriched for multiply mutated single stranded DNA, is transformed into XL10-Gold® ultracompetent cells, where the mutant closed circle ss-DNA is converted into duplex form in vivo. Double stranded plasmid DNA can then be prepared from the transformants and analyzed by appropriate methods to identify clones bearing each of the desired mutations.

The protocol for preparation of this construct is as follows (see FIG. 20). Briefly, the mutant strand synthesis reaction is prepared as indicated in thin-walled tubes. The control reaction contains the following: 2.5 μl of Reaction Buffer (10×); 1 μl each of control template and of control primer mix; 1 μl of dNTP mix; 1 μl of QuikChange Multi enzyme blend; 18.5 μl ddH₂O. The mutagenesis reaction contains 2.5 μl of 10× reaction buffer; 50 ng of ds DNA template for <5 kb, or 100 ng for >5 kg; 100 ng each for 1-3 primers or 50 ng each for 4-5 primers; 1 μl of dNTP mix; 3 μl of QuikSolution reagent; 1 μl of QuikChange Multi enzyme blend; ddH₂O to a final volume of 25 μl. For templates >5 kb, also add 0.75 μl QuikSolution to the reaction. Cycle each reaction using the parameters outlined below. Overlay each reaction mixture with about 30 μl of mineral oil if the thermal cycleter does not have a hot top assembly.

TABLE 7

| Segment | Cycles | Temperature | Time |
|---|---|---|---|
| 1 | 1 | 95° C. | 1 minute |
| 2 | 30 | 95° C. | 1 minute |
| | | 55° C. | 1 minute |
| | | 65° C. | 2 minutes/kb of |
| | | | plasmid |

Then add 1 μl of Dpm 1 restriction enzyme (10 U/μl) below the mineral oil overlay (if used). Next, gently and thoroughly mix each reaction, spin down in a microcentrifuge for 1 minute, and immediately incubate at 37° C. for 1 hour to digest ds-DNA containing parental strands. Lastly transfor 1.5 μl of the Dpn 1-treated DNA from each control and sample reaction in separate 45-μl aliquots of XL10-Gold ultracompetent cells.

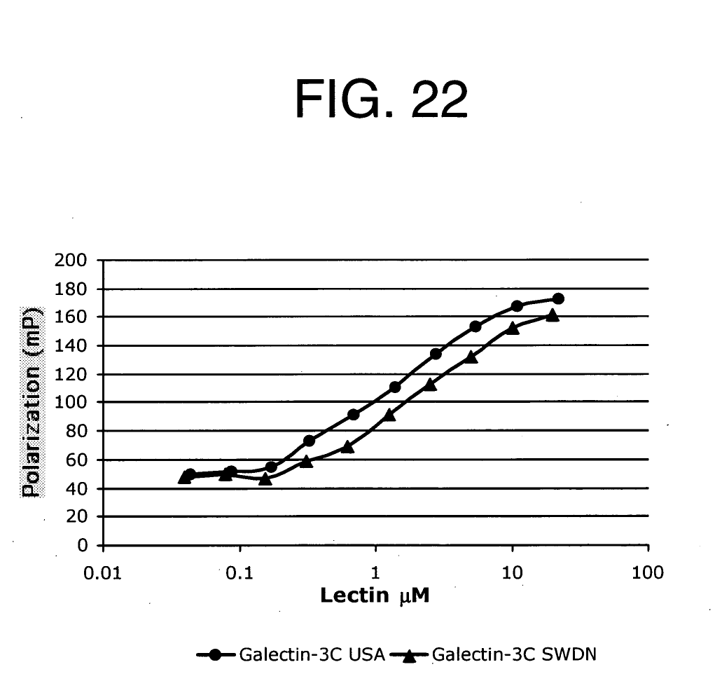

The carbohydrate affinity of the homologues of N-terminally truncated galectin-3 proteins can be compared by fluorescence polarization (FP; see FIG. 22). In FP polarized light is used to excite a fluorescent probe (88). The degree of polarization lost in the emitted light correlates with the mobility of the protein. A small molecule probe rotates slower when bound to a protein and emit more polarized light, so the method permits comparisons of relative affinities and the calculation of the bound and free state in solution without any separation of the phases. Using modified versions of a galectin-3 carbohydrate ligand such as N-acetyllactosamine with a fluorescent tag the affinities of the proteins for carbohydrates can be compared.

For the assay of homotypic cell aggregation, 1×10⁶ cancer cells in a total volume of 1 ml of media containing increasing concentrations of galectin-3 or an N-terminally truncated galectin-3 are placed in 12×75 mm test tubes. The tubes are shaken at 80 rpm for one hour in a 37° C. water bath after which 15-μl of 1% formaldehyde is added to each tube. After 5 minutes of incubation without shaking, the number of single cells in each cell suspension is determined by removing four separate 10-ml aliquots from each tube after swirling and then counting single cells using a hemocytometer and microscope. Assays are performed in triplicate. Data are presented as the percentage of single cells in the test tubes as compared with the untreated control cells. Percents less than 100 are due to increased aggregation whereas those greater than 100 are the result of decreased aggregation.

### Example 6

Rheumatoid arthritis (RA) is a common autoimmune disease affecting about 1% of the human population that is characterized by chronic inflammation of the synovial joints and by subsequent progressive, erosive destruction of articular tissue. The pathogenesis of rheumatoid arthritis is not fully understood but it seems likely that an autoimmune-mediated attack on the joints has a crucial role. Generation of free radicals and other reactive oxygen species such as singlet oxygen and hypochlorous acid that are highly reactive transient chemical species with the potential to initiate cellular damage in joint tissues could have a role in the pathogenesis. The peroxidation of lipids is thought to be a critical mechanism of the injury that occurs. A method of evaluating lipid peroxidation is analysis of tissue malonaldehyde. Collagen-induced arthritis in Lewis rats is a widely used experimental animal model of inflammatory polyarthritis with clinical and pathological features similar to those of human RA that are dependent on both humoral and cellular immunity to the immunizing antigen (147). Investigations have indirectly implicated TNF-α as a contributor to cellular damage in collagen-induced arthritis (reviwed in (147)).

As background for the following example, evidence suggests that in rheumatoid arthritis and juvenile idiopathic arthritis defective apoptosis of mononuclear phagocytic cells and activation of synovial fibroblasts is related to increased expression of galectin-3 (62,63). Therefore, a potential therapeutic agent for arthritis could be based on the inhibition of galectin-3. Soluble recombinant N-terminally truncated galectin-3 competes with endogenous galectin-3 for carbohydrate binding sites in the extracellular matrix and cell-cell adhesions important in cellular invasion process and activation of immune cells through cross-linking carbohydrate expressing receptors on cell surfaces by multimerization mediated by the N-terminal domain. In arthritis N-terminally truncated galectin-3 is expected to be therapeutic by reducing the threshold for induction of apoptosis in some cells of the immune system that express and absorb galectin-3 from the extracellular milieu, and to reduce the activation of other cells such as neutrophils and synovial fibroblasts.

Induction of Adjuvant Arthritis

Male Lewis rats 6-7 weeks old, with a mean weight of 175-200 g, are maintained in a 12 hours light: 12 hours dark cycle. The animals are given standard rodent chow and water ad libitum. One experiment is designed to test the efficacy on rats treated from the day of induction of arthritis compared to rats treated after the first sign of symptoms. Rats are divided into the following groups: (1) control (n=10) vehicle twice daily; (2) collagen-induced arthritis plus vehicle twice daily (PBS with 1 mg/ml lactose; n=10); (3) collagen-induced arthritis plus N-terminally truncated galectin-3 (5 mg/kg in PBS with 1 mg/ml lactose; twice daily from day 1, s.q.; n=10); (4) collagen-induced arthritis plus N-terminally truncated galectin-3 (5 mg/kg in PBS with 1 mg/ml lactose; twice daily from day 11, s.q.; n=10).

A second experiment is designed to test the efficacy of prednisolone (148), a steroid that has anti-inflammatory activity in arthritis, with N-terminally truncated galectin-3 to determine additive or synergistic efficacy. Rats are divided into the following groups (1) vehicle (100 μl peanut oil given i.p. and 150 μl PBS with 1 mg/ml lactose) given s.c. daily; control (n=10); (2) collagen-induced arthritis plus vehicle daily (n=10); (3) collagen-induced arthritis plus N-terminally truncated galectin-3 (5 mg/kg in PBS with 1 mg/ml lactose; daily from day 11, s.q.; n=10); (4) collagen-induced arthritis plus N-terminally truncated galectin-3 (5 mg/kg in PBS with 1 mg/ml lactose s.c.) with prednisolone (4 μmol/kg in peanut oil i.p) once daily from day 11, s.q.; n=10); and (5) collagen-induced arthritis with prednisolone (4 μmol/kg in peanut oil i.p) once daily from day 11, s.q.; n=10.

Bovine type II collagen is purchased from Sigma-Aldrich (St. Louis, Mo.); complete Freund's adjuvant is obtained from Difco Laboratories (Detroit, Mich.). All other reagents are purchased from Fluka (division of Sigma-Aldrich). Collagen-induced arthritis is induced in rats as by multiple intradermal injections, at the base of the tail and into three to five other sites on the back, of 250 μg of bovine type II collagen in 125 μl of 0.1 M acetic acid emulsified in an equal volume of complete Freund's adjuvant containing 2 mg/ml Mycobacterium tuberculosis H37 RA as previously described (147). Rats are challenged again with the same antigen preparation 7 days later. Before injection, animals are anesthetized and injections are performed with a 15-gauge needle. Disease develops about 11 days after the second immunization. Animals are randomized to receive treatment timed either from the day of the first injection to induce arthritis or at day 11 to coincide approximately with the onset of arthritis pathology.

Arthritis Assessments

Evaluation of joint inflammation is performed by a blinded observer who quantifies the severity of the arthritis in each paw daily by a clinical score measurement from 0 to 4 as follows: 0, no macroscopic signs of arthritis; 1, swelling of one group of joints (namely, wrist or ankle joints); 2, two groups of swollen joints; 3, three groups of swollen joints; 4; swelling of the entire paw. The maximum score for each rat is 16. Clinical severity is also assessed by the quantification of the paw volume changes. Measurements are performed with a caliper. Changes in body weight are monitored to determine the rate of the increment in each rat.

Histological Analysis

Anesthetized rats are sacrificed at day 21; the hind limbs are removed and fixed in 10% buffered formalin. The limbs are decalcified in 5% formic acid, processed for paraffin embedding, sectioned at 5-μm thickness, and stained with haematoxylin-eosin for light microscopy. Sections are examined for the presence of hyperplasia of the synovium, pannus formation, and destruction of the joint space.

Lipid Peroxidation Analysis

Determination of malonaldehyde in the articular tissue is performed to estimate the extent of lipid peroxidation in the damaged cartilage. At the end of the experiment hind limbs are removed and maintained at 0° C., then the joint cartilage is quickly separated from the bone and muscular tissue and frozen at −80° C. until assay. On the day of analysis, after thawing, cartilage samples are washed in ice-cold 20 mM Tris-HCl, pH 7.4, and blotted on absorbent paper. Each sample is then minced in ice-cold 20 mM Tris-HCl, pH 7.4 containing 1 mg/ml butylated hydroxytoluene and homogenised in a 1:10 (w/v) ratio with an Ultra-Turrax homogeniser. After centrifugation for 10 minutes at 3000 g and 4° C., the clear supernatant is used for biochemical assay. Analysis is performed with a colorimetric commercial kit (Lipid peroxidation assay kit, cat. no. 437634; Calbiochem, La Jolla, Calif., USA). In brief, 0.65 ml of 10.3 mM N-methyl-2-phenylindole in acetonitrile is added to 0.2 ml of homogenate supernatant. After vortex-mixing for 3-4 seconds and the addition of 0.15 ml of 37% HCl, samples are well mixed, closed with a tight stopper and incubated for 60 minutes at 45° C. The samples are then cooled on ice and the absorbance is measured spectrophotometrically at 586 nm. A calibration curve of an accurately prepared standard malonaldehyde solution is also run for quantification. The malonaldehyde concentration is expressed as nmol/mg of protein.

Evaluation of Superoxide Dismutase (SOD)

Samples of joint cartilage are washed with 0.9% NaCl containing 0.16 mg/ml heparin and homogenized in ice-cold 0.25 M sucrose containing 1 mM diethylenetriamine pentaacetic acid (1:1, w/v). Each sample is then centrifuged for 20 minutes at 20,000 g and 4° C. The supernatant is aspirated and the total superoxide dismutase activity is assayed spectrophotometrically at 505 nm with a commercial kit (Ransod assay kit, Cat. No. Sd 125; Randox Laboratories, Crumlin, UK). In brief, 50 μl of each diluted sample (1:10 [w/v] in 0.01 M potassium phosphate buffer, pH 7.0) is mixed with 1.7 ml of solution containing 0.05 mM xanthine and 0.025 mM iodonitrotetra-zolium chloride. After being mixed for 5 seconds, 250 μl of xanthine oxidase (80 U/l) is added. The time between reading the initial and final absorbances is 3 minutes. A standard curve of SOD solution (from 2 to 32 U/ml) is run for quantification. All standard and diluted sample rates are converted into a percentage of the buffer diluent rate and subtracted from 100% to give a percentage inhibition. Sample SOD activities are obtained from a plotted curve of the percentage inhibition for each standard. SOD values are expressed as units/mg of protein.

Plasma Tumour Necrosis Factor-α (TNF-α) Assay

Plasma TNF-α concentration is determined with an ELISA commercial kit (Rat TNF-α ELISA kit, Cat. no. 22079; NBS Biologicals Ltd, Huntingdon, UK). At the end of the experiment, samples of blood (0.5 ml) are drawn from a tail vessel. The blood is collected in polyethylene tubes with the previous addition of 25 μl of heparin solution (4000 IU). The plasma samples obtained after centrifugation for 10 minutes at 3000 g and 4° C. are frozen at −80° C. until assay. In brief, 100 μl of standards, samples and controls are added to each well of the coated microplate. After 3 hours of incubation at 24° C. the microplate is decanted and the liquid discarded. Then, 100 μl of biotinylated anti-TNF-α antibody is added to each well. After 45 minutes of incubation at 24° C. and a further elimination of the liquid from the wells, 100 μl of streptavidin-horseradish peroxidase conjugate is added. After incubation for a further 45 minutes and washing of the wells, 100 μl of chromogen are added. The absorbance of each well was read spectrophotometrically at 450 nm. TNF-α values are expressed as ng/ml.

Articular Neutrophil Accumulation

Myeloperoxidase activity is analysed as an index of neutrophil infiltration in the synovial tissue, because it is closely correlated with the number of neutrophils present in the tissue. Myeloperoxidase in the synovial tissue of joints is measured by a specific assay for this enzyme. Synovial tissue samples are separated from rat joints and are first homogenised in a solution containing 20 mM potassium phosphate buffer, pH 7.0, to 1:10 (w/v) and then centrifuged for 30 minutes at 20,000 g and 4° C. The supernatant of each sample was discarded and the resulting pellet was added to a buffer solution consisting of 0.5% hexadecyltrimethylammonium bromide dissolved in 50 mM potassium phosphate buffer, pH 6, containing 50 μl of protease and phosphatase inhibitor cocktails. Samples are then sonicated for 1 minute and centrifuged for 30 minutes at 20,000 g and 4° C. An aliquot of the supernatant is allowed to react with a solution of o-dianisidine dihydrochloride (0.167 mg/ml) and 0.0005% hydrogen peroxide. The rate of change in absorbance is measured spectrophotometrically at 405 nm. Myeloperoxidase activity has been defined as the concentration of enzyme degrading 1 μmol of peroxide/minutes at 37° C. and is expressed as U/g of protein.

Analyses of Results

Differences between groups were statistically evaluated by Student's paired t test (p<0.05). A characteristic of arthritic joints in rats with collagen-induced arthritis is synovial hyperplasia, pannus formation, exudation of cells into the joint space, and erosion of bone and cartilage. A massive influx of inflammatory cells, synovial hyperplasia, and accumulation of abundant monomorphonuclear and polymorphonuclear cells can be seen. Determination of malonaldehyde in the articular cartilage is performed to estimate free-radical damage to biological membranes. Low levels of malonaldehyde are seen in the control group at the end of the experiment (day 21). In contrast, a significant increase in malonaldehyde production is found in the joints of collagen-induced arthritis rats given vehicle alone.

SOD activity is evaluated to estimate endogenous defenses against superoxide anions. In control animals normal SOD activities range between 10.0 and 15.0 U/mg of protein. In contrast, a significant decrease in this antioxidant is seen in collagen-induced arthritis rats treated only with vehicle. In the control group normal levels of TNF-α are between 20.0 and 40.0 pg/ml. Marked increase in TNF-α concentration is found in the plasma of collagen-induced arthritis rats given vehicle alone. Low myeloperoxidase activity is measured in control rats, and elevated myeloperoxidase levels are measured in the vehicle-administered group. Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

```
LOCUS HUMLAMBP 818 bp mRNA PRI 27-APR-1993
DEFINITION Human non-integrin laminin-binding protein mRNA, complete cds.
ACCESSION M36682
VERSION M36682.1 GI:186921
SOURCE Human breast carcinoma ZR-75-1, cDNA to mRNA, (library of Clontech
        HL1059a), clones 29K34-43.
  ORGANISM Homo sapiens
        Eukaryota; Metazoa; Chordata; Craniata; Vertebrata; Euteleostomi;
        Mammalia; Eutheria; Primates; Catarrhini; Hominidae; Homo.
REFERENCE 1 (bases 1 to 818)
  AUTHORS Oda, Y., Leffler, H., Sakakura, Y., Kasai, K.-i. and Barondes, S. H.
  TITLE Human breast carcinoma cDNA encoding a galactoside-binding lectin
        homologous to mouse Mac-2 antigen, Gene 99, 279-283 (1991)
Author address: Y.Oda,
          University of California San Francisco, LPPI
          Box Bio-0984,
          401 Parnassus Avenue,
          San Francisco, CA 94143.
FEATURES Location/Qualifiers source 1..818
            /organism=“Homo sapiens”
            /db_xref=“taxon:9606”
            /cell_line=“ZR-75-1”
            /tissue_type=“breast carcinoma” CDS 34..786
            /note=“laminin-binding protein”
            /codon_start=1
            /protein_id=“AAA36163.1”
            /db_xref=“GI:186922”
/translation=“MADNFSLHDALSGSGNPNPQGWPGAWGNQPAGAGGYPGASYPGAYP
  GQAPPGAYPGQAPPGAYHGAPGAYPGAPAPGVYPGPPSGPGAYPSSGQPSAPGAYP
  ATGPYGAPAGPLIVPYNLPLPGGVVPRMLITILGTVKPNANRIALDFQRGNDVAFHFNPR
  FNENNRRVIVCNTKLDNNWGREERQSVFPFESGKPFKIQVLVEPDHFKVAVNDAHL
  LQYNHRVKKLNEISKLGISGDIDLTSASYTMI”
  BASE COUNT 215 a 222 c 212 g 169 t
  ORIGIN
      1 agcagccgtc cggagccagc caacgagcgg aaaatggcag acaatttttc gctccatgat
      61 gcgttatctg ggtctggaaa cccaaaccct caaggatggc ctggcgcatg ggggaaccag
    121 cctgctgggg cagggggcta cccaggggct tcctatcctg gggcctaccc cgggcaggca
    181 cccccagggg cttatcctgg acaggcacct ccaggcgcct accatggagc acctggagct
    241 tatcccggag cacctgcacc tggagtctac ccagggccac ccagcggccc tggggcctac
    301 ccatcttctg gacagccaag tgcccccgga gcctaccctg ccactggccc ctatggcgcc
    361 cctgctgggc cactgattgt gccttataac ctgcctttgc ctgggggagt ggtgcctcgc
    421 atgctcataa caattctggg cacggtgaag cccaatgcaa acagaattgc tttagatttc
    481 caaagaggga atgatgttgc cttccacttt aacccacgct tcaatgagaa caacaggaga
    541 gtcattgttt gcaatacaaa gctggataat aactggggaa gggaagaaag acagtcggtt
    601 ttcccatttg aaagtgggaa accattcaaa atacaagtac tggttgaacc tgaccacttc
    661 aaggttgcag tgaatgatgc tcacttgttg cagtacaatc atcgggttaa aaaactcaat
    721 gaaatcagca aactgggaat ttctggtgac atagacctca ccagtgcttc atataccatg
    781 atataatctg aaaggggcag attaaaaaaa aaaacgga
```

### REFERENCES

1. Barondes S H, Cooper D N, Gitt M A, Leffler H. Galectins. Structure and function of a large family of animal lectins. J Biol Chem 1994;269(33):20807-10.
2. Barondes S H, Castronovo V, Cooper D N, Cummings R D, Drickamer K, Feizi T, Gitt M A, Hirabayashi J, Hughes C, Kasai K and others. Galectins: a family of animal beta-galactoside-binding lectins [letter]. Cell 1994;76(4):597-8.
3. Raz A, Lotan R. Lectin-like activities associated with human and murine neoplastic cells. Cancer Res 1981 ;41 (9 Pt 1):3642-7.
4. Raz A, Pazerini G, Carmi P. Identification of the metastasis-associated, galactoside-binding lectin as a chimeric gene product with homology to an IgE-binding protein. Cancer Res 1989;49(13):3489-93.
5. Ochieng J, Platt D, Tait L, Hogan V, Raz T, Carmi P, Raz A. Structure-function relationship of a recombinant human galactoside-binding protein. Biochemistry 1993;32(16):4455-60.
6. Mehul B, Bawumia S, Martin S R, Hughes R C. Structure of baby hamster kidney carbohydrate-binding protein CBP30, an S-type animal lectin. J Biol Chem 1994;269(27): 18250-8.
7. Oda Y, Leffler H, Sakakura Y, Kasai K, Barondes S H. Human breast carcinoma cDNA encoding a galactoside-binding lectin homologous to mouse Mac-2 antigen. Gene 1991 ;99(2):279-83.
8. Seetharaman J, Kanigsberg A, Slaaby R, Leffler H, Barondes S H, Rini J M. X-ray crystal structure of the human galectin-3 carbohydrate recognition domain at 2.1-A resolution. J Biol Chem 1998;273(21):13047-52.
9. Moriki T, Kuwabara I, Liu F T, Maruyama I N. Protein domain mapping by lambda phage display: the minimal lactose-binding domain of galectin-3. Biochem Biophys Res Commun 1999;265(2):291-6.
10. Leffler H, Barondes S H. Specificity of binding of three soluble rat lung lectins to substituted and unsubstituted mammalian beta-galactosides. J Biol Chem 1986;261:10119-10126.
11. Sparrow C P, Leffler H, Barondes S H. Multiple soluble beta-galactoside-binding lectins from human lung. J Biol Chem 1987;262(15):7383-90.
12. Leffler H. Introduction to galectins. Trends Glycosci Glycotechnol 1997;45:9-19.
13. Perillo N L, Pace K E, Seilhamer J J, Baum L G. Apoptosis of T-cells mediated by galectin-1. Nature 1995;378:736-738.
14. Kaltner H, Lips K S, Lippert R G, Sinowatz F, Gabius H J. Quantitation and histochemical localization of galectin-1 and galectin-1-reactive glycoconjugates in fetal development of bovine organs. Histol Histopathol 1997; 12(4):945-960.
15. Rabinovich G A. Galectins: an evolutionarily conserved family of animal lectins with multifunctional properties; a trip from the gene to clinical therapy. Cell Death Differ 1999;6(8):711-21.
16. Rabinovich G A, Riera C M, Landa C A, Sotomayor C E. Galectins: a key intersection between glycobiology and immunology. Braz J Med Biol Res 1999;32(4):383-93.
17. Rabinovich G A, Baum L G, Tinari N, Paganelli R, Natoli C, Liu F T, lacobelli S. Galectins and their ligands: amplifiers, silencers or tuners of the inflammatory response? Trends Immunol 2002;23(6):313-20.
18. Rabinovich G, Rubinstein N, Toscano M. Role of galectins in inflammatory and immunomodulatory processes. Biochim Biophys Acta 2002;1572(2-3):274.
19. Rabinovich G A, Rubinstein N, Fainboim L. Unlocking the secrets of galectins: a challenge at the frontier of glyco-immunology. J Leukoc Biol 2002;71 (5):741-52.
20. Gaudin J C, Mehul B, Hughes R C. Nuclear localisation of wild type and mutant galectin-3 in transfected cells. Biol Cell 2000;92(1):49-58.
21. Openo K P, Kadrofske M M, Patterson R J, Wang J L. Galectin-3 expression and subcellular localization in senescent human fibroblasts. Exp Cell Res 2000;255(2):278-90.
22. Yu F, Finley R L, Jr., Raz A, Kim H R. Galectin-3 Translocates to the Perinuclear Membranes and Inhibits Cytochrome c Release from the Mitochondria. A ROLE FOR SYNEXIN IN GALECTIN-3 TRANSLOCATION. J Biol Chem 2002;277(18):15819-27.
23. Prochiantz A. Messenger proteins: homeoproteins, TAT and others. Curr Opin Cell Biol 2000;12(4):400-6.
24. Massa S M, Cooper D N, Leffler H, Barondes S H. L-29, an endogenous lectin, binds to glycoconjugate ligands with positive cooperativity. Biochemistry 1993;32(1):260-7.
25. Liu F T, Hsu D K, Zuberi R I, Hill P N, Shenhav A, Kuwabara I, Chen S S. Modulation of functional properties of galectin-3 by monoclonal antibodies binding to the non-lectin domains. Biochemistry 1996;35(19):6073-9.
26. Hsu DK, Suberi R I, Liu F T. Biochemical and biophysical characterization of human recombinant IgE-binding protein, an S-type animal lectin. J. Biol. Chem. 1992;267:14167-14174.
27. Gong H C, Honjo Y, Nangia-Makker P, Hogan V, Mazurak N, Bresalier R S, Raz A. The NH2 terminus of galectin-3 governs cellular compartmentalization and functions in cancer cells. Cancer Res 1999;59(24):6239-45.
28. Yamaoka A, Kuwabara I, Frigeri L G, Liu F T. A human lectin, galectin-3 (epsilon bp/Mac-2), stimulates superoxide production by neutrophils. J Immunol 1995;154(7):3479-87.
29. Kuwabara I, Liu F T. Galectin-3 promotes adhesion of human neutrophils to laminin. J Immunol 1996;156(10):3939-44.
30. Itzkowitz S H. Galectins: multipurpose carbohydrate-binding proteins implicated in tumor biology. Gastroenterology 1997;1 13(6):2003-5.
31. Ochieng J, Warfield P. Galectin-3 binding potentials of mouse tumor EHS and human placental laminins. Biochem Biophys Res Commun 1995;217(2):402-6.
32. Ochieng J, Warfield P, Green-Jarvis B, Fentie I. Galectin-3 regulates the adhesive interaction between breast carcinoma cells and elastin. J Cell Biochem 1999;75(3):505-14.
33. Ochieng J, Leite-Browning M L, Warfield P. Regulation of cellular adhesion to extracellular matrix proteins by galectin-3. Biochem Biophys Res Commun 1998;246(3):788-91.
34. Inohara H, Raz A. Functional evidence that cell surface galectin-3 mediates homotypic cell adhesion. Cancer Res 1995;55(15):3267-71.
35. Hirabayashi J, Hashidate T, Arata Y, Nishi N, Nakamura T, Hirashima M, Urashima T, Oka T, Futai M, Muller W and others. Oligosaccharide specificity of galectins: a search by frontal affinity chromatography. Biochim Biophys Acta 2002; 1572(2-3):232.
36. Dennis J W, Granovsky M, Warren C E. Glycoprotein glycosylation and cancer progression. Biochim Biophys Acta 1999; 1473(1):21-34.
37. Granovsky M, Fata J, Pawling J, Muller W J, Khokha R, Dennis J W. Suppression of tumor growth and metastasis in Mgat5-deficient mice. Nat Med 2000;6(3):306-12.
38.. Couldrey C, Green J E. Metastases: the glycan connection. Breast Cancer Res 2000;2(5):321-3.
39. Raz A, Zhu D G, Hogan V, Shah N, Raz T, Karkash R, Pazerini G, Carmi P. Evidence for the role of 34-kDa galactoside-binding lectin in transformation and metastasis. Int J Cancer 1990;46(5):871-7.
40. Nangia-Makker P, Honjo Y, Sarvis R, Akahani S, Hogan V, Pienta K J, Raz A. Galectin-3 induces endothelial cell morphogenesis and angiogenesis. Am J Pathol 2000;1 56(3):899-909.
41. Bresalier R S, Mazurek N, Sternberg L R, Byrd J C, Yunker C K, Nangia-Makker P, Raz A. Metastasis of human colon cancer is altered by modifying expression of the beta-galactoside-binding protein galectin 3. Gastroenterology 1998; 115(2):287-96.
42. Nangia-Makker P, Sarvis R, Visscher D W, Bailey-Penrod J, Raz A, Sarkar F H. Galectin-3 and L1 retrotransposons in human breast carcinomas. Breast Cancer Res Treat 1998;49(2):171-83.
43. Lotz M M, Andrews C W, Jr., Korzelius C A, Lee E C, Steele G D, Jr., Clarke A, Mercurio A M. Decreased expression of Mac-2 (carbohydrate binding protein 35) and loss of its nuclear localization are associated with the neoplastic progression of colon carcinoma. Proc Natl Acad Sci USA 1993;90(8):3466-70.
44. Castronovo V, Van Den Brule F A, Jackers P, Clausse N, Liu F T, Gillet C, Sobel M E. Decreased expression of galectin-3 is associated with progression of human breast cancer. J Pathol 1996;179(1):43-8.
45. Idikio H. Galectin-3 expression in human breast carcinoma: correlation with cancer histologic grade. Int J Oncol 1998;12(6):1287-90.
46. Andre S, Kojima S, Yamazaki N, Fink C, Kaltner H, Kayser K, Gabius H J. Galectins-1 and -3 and their ligands in tumor biology. Non-uniform properties in cell-surface presentation and modulation of adhesion to matrix glycoproteins for various tumor cell lines, in biodistribution of free and liposome-bound galectins and in their expression by breast and colorectal carcinomas with/without metastatic propensity. J Cancer Res Clin Oncol 1999; 125(8-9):461-74.
47. Yang R-Y, Hsu D, Liu F-T. Expression of galectin-3 modulates T-cell growth and apoptosis. Proc Natl Acad Sci USA 1996;93:6737-6742.
48. Akahani S, Nangia-Makker P, Inohara H, Kim H R, Raz A. Galectin-3: a novel antiapoptotic molecule with a functional. BH1 (NWGR) domain of Bcl-2 family. Cancer Res 1997;57(23):5272-6.
49. Kim H R, Lin H M, Biliran H, Raz A. Cell cycle arrest and inhibition of anoikis by galectin-3 in human breast epithelial cells. Cancer Res 1999;59(16):4148-54.
50. Matarrese P, Fusco O, Tinari N, Natoli C, Liu F T, Semeraro M L, Malorni W, lacobelli S. Galectin-3 overexpression protects from apoptosis by improving cell adhesion properties. Int J Cancer 2000;85(4):545-54.
51. Matarrese P, Tinari N, Semeraro M, Natoli C, lacobelli S, Malorni W. Galectin-3 overexpression protects from cell damage and death by influencing mitochrondrial homeostatis. FEBS letters 2000;473:311-315.
52. Pienta K J, Naik H, Akhtar A, Yamazaki K, Replogle T S, Lehr J, Donat T L, Tait L, Hogan V, Raz A. Inhibition of spontaneous metastasis in a rat prostate cancer model by oral administration of modified citrus pectin [see comments]. J Natl Cancer Inst 1995;87(5):348-53.
53. Joo H G, Goedegebuure P S, Sadanaga N, Nagoshi M, von Bernstorff W, Eberlein T J. Expression and function of galectin-3, a beta-galactoside-binding protein in activated T lymphocytes. J Leukoc Biol 2001 ;69(4):555-64.
54. Truong M J, Gruart V, Kusnierz J P, Papin J P, Loiseau S, Capron A, Capron M. Human neutrophils express immunoglobulin E (IgE)-binding proteins (Mac-2/epsilon BP) of the S-type lectin family: role in IgE-dependent activation. J Exp Med 1993;177(1):243-8.
55. Liu F T, Hsu D K, Zuberi R I, Kuwabara I, Chi E Y, Henderson W R, Jr. Expression and function of galectin-3, a beta-galactoside-binding lectin, in human monocytes and macrophages. Am J Pathol 1995;147(4):1016-28.
56. Pesheva P, Urschel S, Frei K, Probstmeier R. Murine microglial cells express functionally active galectin-3 in vitro. J Neurosci Res 1998;51(1):49-57.
57. Sano H, Hsu D K, Yu L, Apgar J R, Kuwabara I, Yamanaka T, Hirashima M, Liu F T. Human galectin-3 is a novel chemoattractant for monocytes and macrophages. J Immunol 2000;165(4):2156-64.
58. Walther M, Kuklinski S, Pesheva P, Guntinas-Lichius O, Angelov DN, Neiss W F, Asou H, Probstmeier R. Galectin-3 is upregulated in microglial cells in response to ischemic brain lesions, but not to facial nerve axotomy. J Neurosci Res 2000;61 (4):430-5.
59. Kimata H. Enhancement of IgE Production in B Cells by Neutrophils via Galectin-3 in IgE-Associated Atopic Eczema/Dermatitis Syndrome. Int Arch Allergy Immunol 2002;128(2):168-70.
60. Villa-Verde D M, Silva-Monteiro E, Jasiulionis M G, Farias-De-Oliveira D A, Brentani R R, Savino W, Chammas R. Galectin-3 modulates carbohydrate-dependent thymocyte interactions with the thymic microenvironment. Eur J Immunol 2002;32(5):1434-1444.
61. Demetriou M, Granovsky M, Quaggin S, Dennis J W. Negative regulation of T-cell activation and autoimmunity by Mgat5 N-glycosylation. Nature 2001 ;409(6821):733-9.
62. Harjacek M, Diaz-Cano S, De Miguel M, Wolfe H, Maldonado C A, Rabinovich G A. Expression of galectins-1 and -3 correlates with defective mononuclear cell apoptosis in patients with juvenile idiopathic arthritis. J Rheumatol 2001 ;28(8): 1914-22.
63. Ohshima S, Kuchen S, Seemayer C A, Kyburz D, Hirt A, Klinzing S, Michel B A, Gay R E, Liu F T, Gay S and others. Galectin 3 and its binding protein in rheumatoid arthritis. Arthritis Rheum 2003;48(10):2788-95.
64. Lu J, Teh C, Kishore U, Reid K B. Collectins and ficolins: sugar pattern recognition molecules of the mammalian innate immune system. Biochim Biophys Acta 2002; 1572(2-3):387-400.
65. Truong M J, Liu F T, Capron M. Human granulocytes express functional IgE-binding molecules, Mac-2/epsilon BP. Ann N Y Acad Sci 1994;725(3):234-46.
66. Feuk-Lagerstedt E, Jordan E T, Leffler H, Dahlgren C, Karisson A. Identification of CD66a and CD66b as the major galectin-3 receptor candidates in human neutrophils. J Immunol 1999;163(10):5592-8.
67. Sato S, Ouellet N, Pelletier I, Simard M, Rancourt A, Bergeron M G. Role of galectin-3 as an adhesion molecule for neutrophil extravasation during streptococcal pneumonia. J Immunol 2002;168(4):1813-22.
68. Karlsson A, Follin P, Leffler H, Dahlgren C. Galectin-3 activates the NADPH-oxidase in exudated but not peripheral blood neutrophils. Blood 1998;91 (9):3430-8.
69. Almkvist J, Faldt J, Dahigren C, Leffler H, Karlsson A. Lipopolysaccharide-induced gelatinase granule mobilization primes neutrophils for activation by galectin-3 and formylmethionyl-Leu-Phe. Infect Immun 2001;69(2):832-7.
70. Jeng K C, Frigeri L G, Liu F T. An endogenous lectin, galectin-3 (epsilon BP/Mac-2), potentiates IL-1 production by human monocytes. Immunol Lett 1994;42(3):113-6.
71. Sano H, Hsu D K, Apgar J R, Yu L, Sharma B B, Kuwabara I, lzui S, Liu F T. Critical role of galectin-3 in phagocytosis by macrophages. J Clin Invest 2003;1 12(3):389-97.
72. Colnot C, Ripoche M A, Milon G, Montagutelli X, Crocker P R, Poirier F. Maintenance of granulocyte numbers during acute peritonitis is defective in galectin-3-null mutant mice. Immunology 1998;94(3):290-6.
73. Hsu D K, Yang R Y, Pan Z, Yu L, Salomon D R, Fung-Leung W P, Liu F T. Targeted disruption of the galectin-3 gene results in attenuated peritoneal inflammatory responses. Am J Pathol 2000;156(3):1073-83.
74. Vlassara H, Li Y M, lmani F, Wojciechowicz D, Yang Z, Liu F T, Cerami A. Identification of galectin-3 as a high-affinity binding protein for advanced glycation end products (AGE): a new member of the AGE-receptor complex. Mol Med 1995; 1 (6):634-46.
75. Pricci F, Leto G, Amadio L, lacobini C, Romeo G, Cordone S, Gradini R, Barsotti P, Liu F T, Di Mario U and others. Role of galectin-3 as a receptor for advanced glycosylation end products. Kidney Int Suppi 2000;77:S31-9.
76. Pugliese G, Pricci F, Leto G, Amadio L, lacobini C, Romeo G, Lenti L, Sale P, Gradini R, Liu F T and others. The diabetic milieu modulates the advanced glycation end product-receptor complex in the mesangium by inducing or upregulating galectin-3 expression. Diabetes 2000;49(7):1249-57.
77. Zhu W, Sano H, Nagai R, Fukuhara K, Miyazaki A, Horiuchi S. The role of galectin-3 in endocytosis of advanced glycation end products and modified low density lipoproteins. Biochem Biophys Res Commun 2001;280(4):1183-8.
78. Pugliese G, Pricci F, lacobini C, Leto G, Amadio L, Barsotti P, Frigeri L, Hsu D K, Vlassara H, Liu F T and others. Accelerated diabetic glomerulopathy in galectin-3/AGE receptor 3 knockout mice. Faseb J 2001;15(13):2471-9.
79. John C M, Jarvis G A, Swanson K V, Leffler H, Cooper M D, Huflejt M E, Griffiss J M. Galectin-3 binds lactosaminylated lipooligosaccharides from Neisseria gonorrhoeae and is selectively expressed by mucosal epithelial cells that are infected. Cell Microbiol 2002;4(10):649-62.
80. Sato S, Hughes R C. Binding specificity of a baby hamster kidney lectin for H type I and II chains, polylactoamine glycans and appropriately glycosylated forms of laminin and fibronectin. J Biol Chem 1992;267: 983-6990.
81. Kuklinski S, Probstmeier R. Homophilic binding properties of galectin-3: involvement of the carbohydrate recognition domain. J Neurochem 1998;70(2):814-23.
82. Andre S, Frisch B, Kaltner H, Desouza D L, Schuber F, Gabius H J. Lectin-mediated drug targeting: selection of valency, sugar type (Gal/Lac), and spacer length for cluster glycosides as parameters to distinguish ligand binding to C-type asialoglycoprotein receptors and galectins. Pharm Res 2000; 17(8):985-90.
83. Lee R T, Lee Y C. Affinity enhancement by multivalent lectin-carbohydrate interaction. Glycoconj J 2000;17(7-9):543-51.
84. Barboni E A, Bawumia S, Henrick K, Hughes R C. Molecular modeling and mutagenesis studies of the N-terminal domains of galectin-3: evidence for participation with the C-terminal carbohydrate recognition domain in oligosaccharide binding [In Process Citation]. Glycobiology 2000; 10(11): 1201-8.
85. Ochieng J, Green B, Evans S, James O, Warfield P. Modulation of the biological functions of galectin-3 by matrix metalloproteinases. Biochim Biophys Acta 1998; 1379(1):97-106.
86. Ochieng J, Fridman R, Nangia-Makker P, Kleiner D E, Liotta L A, Stetler-Stevenson W G, Raz A. Galectin-3 is a novel substrate for human matrix metalloproteinases-2 and -9. Biochemistry 1994;33(47): 14109-14.
87. Yang R Y, Hill P N, Hsu D K, Liu F T. Role of the carboxyl-terminal lectin domain in self-association of galectin-3. Biochemistry 1998;37(12):4086-92.
88. Sorme P, Kahl-Knutson B, Wellmar U, Nilsson U J, Leffler H. Fluorescence polarization to study galectin-ligand interactions. Methods Enzymol 2003;362:504-12.
89. Inohara H, Akahani S, Koths K, Raz A. Interactions between galectin-3 and Mac-2-binding protein mediate cell-cell adhesion. Cancer Res 1996;56(19):4530-4.
90. Gaberc-Porekar V, Menart V. Perspectives of immobilized-metal affinity chromatography. J. Biochem. Biophys. Methods 2001 ;49:335-360.
91. Cunningham B C, Wells J A. High-resolution epitope mapping of hGH-receptor interactions by alanine-scanning mutagenesis. Science 1989;244(4908): 1081-5.
92. Smith G P. Surface presentation of protein epitopes using bacteriophage expression systems. Curr Opin Biotechnol 1991;2(5):668-73.
93. Clackson T, Wells J A. In vitro selection from protein and peptide libraries. Trends Biotechnol 1994; 12(5): 173-84.
94. Manning M C, Patel K, Borchardt R T. Stability of protein pharmaceuticals. Pharm Res 1989;6(11):903-18.
95. Costantino H R, Firouzabadian L, Hogeland K, Wu C, Beganski C, Carrasquillo K G, Cordova M, Griebenow K, Zale S E, Tracy M A. Protein spray-freeze drying. Effect of atomization conditions on particle size and stability. Pharm Res 2000; 17(11):1374-83.
96. Putney S, Burke P. Improving protein therapeutics with sustained-release formulations. Nat Biotechnol 1998;16:153-157.
97. Morita T, Horikiri Y, Yamahara H, Suzuki T, Yoshino H. Formation and isolation of spherical fine protein microparticles through lyophilization of protein-poly(ethyiene glycol) aqueous mixture. Pharm Res 2000;17(11):1367-73.
98. Cleland J L, Daugherty A, Mrsny R. Emerging protein delivery methods. Curr Opin Biotechnol 2001;12(2):212-9.
99. Abuchowski A, McCoy J R, Palczuk N C, van Es T, Davis F F. Effect of covalent attachment of polyethylene glycol on immunogenicity and circulating life of bovine liver catalase. J Biol Chem 1977;252(11):3582-6.
100. Chinol M, Casalini P, Maggiolo M, Canevari S, Omodeo E S, Caliceti P, Veronese F M, Cremonesi M, Chiolerio F, Nardone E and others. Biochemical modifications of avidin improve pharmacokinetics and biodistribution, and reduce immunogenicity. Br J Cancer 1998;78(2):189-97.
101. Veronese F M. Peptide and protein PEGylation: a review of problems and solutions. Biomaterials 2001 ;22(5):405-17.
102. Veronese F M, Sacca B, Polverino de Laureto P, Sergi M, Caliceti P, Schiavon O, Orsolini P. New PEGs for peptide and protein modification, suitable for identification of the PEGylation site. Bioconjug Chem 2001;12(1):62-70.
103. Hurwitz E, Klapper L N, Wilchek M, Yarden Y, Sela M. Inhibition of tumor growth by poly(ethylene glycol) derivatives of anti-ErbB2 antibodies. Cancer Immunol Immunother 2000;49(4-5):226-34.
104. Lee L S, Conover C, Shi C, Whitlow M, Filpula D. Prolonged circulating lives of single-chain Fv proteins conjugated with polyethylene glycol: a comparison of conjugation chemistries and compounds. Bioconjug Chem 1999;10(6):973-81.
105. Kuan C T, Wang Q C, Pastan I. Pseudomonas exotoxin A mutants. Replacement of surface exposed residues in domain II with cysteine residues that can be modified with polyethylene glycol in a site-specific manner. J Biol Chem 1994;269(10):7610-6.
106. Barboni E A, Bawumia S, Hughes R C. Kinetic measurements of binding of galectin 3 to a laminin substratum. Glycoconj J 1999;16(7):365-73.
107. Feizi T, Solomon J C, Yuen C T, Jeng K C, Frigeri L G, Hsu D K, Liu F T. The adhesive specificity of the soluble human lectin, IgE-binding protein, toward lipid-linked oligosaccharides. Presence of the blood group A, B, B-like, and H monosaccharides confers a binding activity to tetrasaccharide (lacto-N-tetraose and lacto-N-neotetraose) backbones. Biochemistry 1994;33(20):6342-9.
108. Safa M M, Foon K A. Adjuvant immunotherapy for melanoma and colorectal cancers. Semin Oncol 2001 ;28(1):68-92.
109. Slovin S F, Scher H I. Peptide and carbohydrate vaccines in relapsed prostate cancer: immunogenicity of synthetic vaccines in man--clinical trials at Memorial Sloan-Kettering Cancer Center. Semin Oncol 1999;26(4):448-54.
110. Rohrbach F, Gerstmayer B, Biburger M, Wels W. Construction and characterization of bispecific costimulatory molecules containing a minimized CD86 (B7-2) domain and single-chain antibody fragments for tumor targeting. Clin Cancer Res 2000;6(11):4314-22.
111. Kriangkum J, Xu B, Gervais C, Paquette D, Jacobs F A, Martin L, Suresh M R. Development and characterization of a bispecific single-chain antibody directed against T-cells and ovarian carcinoma. Hybridoma 2000;19(1):33-41.
112. Thirion S, Motmans K, Heyligen H, Janssens J, Raus J, Vandevyver C. Mono- and bispecific single-chain antibody fragments for cancer therapy. Eur J Cancer Prev 1996;5(6):507-11.
113. Testoni N, Martinelli G, Farabegoli P, Zaccaria A, Amabile M, Raspadori D, Pelliconi S, Zuffa E, Carboni C, Tura S. A new method of “in-cell reverse transcriptase-polymerase chain reaction” for the detection of BCR/ABL transcript in chronic myeloid leukemia patients. Blood 1996;87(9):3822-7.
114. Huston J S, Mudgett-Hunter M, Tai M S, McCartney J, Warren F, Haber E, Oppermann H. Protein engineering of single-chain Fv analogs and fusion proteins. Methods Enzymol 1991 ;203:46-88.
115. Johnson S, Bird R E. Construction of single-chain Fv derivatives monoclonal antibodies and their production in Escherichia coli. Methods Enzymol 1991;203:88-98.
116. Martin A N, Swarbrick J, Cammarata A. Physical Pharmacy. Philadelphia: Lea & Febiger; 1996.
117. Rowland M, Tozer T N. Clinical Pharmacokinetics. Baltimore: Williams & Wilkins; 1995.
118. Glinsky V V, Huflejt M E, Glinsky G V, Deutscher S L, Quinn T P. Effects of Thomsen-Friedenreich antigen-specific peptide P-30 on beta-galactoside-mediated homotypic aggregation and adhesion to the endothelium of MDA-MB-435 human breast carcinoma cells. Cancer Res 2000;60(10):2584-8.
119. Yang M, Baranov E, Jiang P, Sun F-X, Li X-M, Hasegawa S, Bouvet M, Al-Tuwaijri M, Chishima T, Shimada H and others. Whole-body optical imaging of green fluorescent protein-expressing tumors and metastases. Proc Natl Acad Sci USA 2000;97:1206-1211.
120. Zar J H. Biostatistical Analysis. Englewood Cliffs, N.J.: Prentice-Hall; 1996.
121. Hoffman R M. Orthotopic metastatic mouse models for anticancer drug discovery and evaluation: a bridge to the clinic. Invest New Drugs 1999; 17(4):343-59.
122. Abrams J S. Adjuvant therapy for breast cancer--results from the USA consensus conference. Breast Cancer 2001 ;8(4):298-304.
123. Going J J, Mallon E A, Leake R E, Bartlett J M, Gusterson B A. What the clinician needs from the pathologist: evidence-based reporting in breast cancer. Eur J Cancer 2001 ;37(Suppl 7):S5-17.
124. Chew H K. Medical management of breast cancer: today and tomorrow. Cancer Biother Radiopharm 2002;17(2):137-49.
125. Reddy K R. Controlled-release, pegylation, liposomal formulations: new mechanisms in the delivery of injectable drugs. Ann Pharmacother 2000;34(7-8):915-23.
126. Delgado C, Francis G E, Fisher D. The uses and properties of PEG-linked proteins. Crit Rev Ther Drug Carrier Syst 1992;9(3-4):249-304.
127. Harris J M, Martin N E, Modi M. Pegylation: a novel process for modifying pharmacokinetics. Clin Pharmacokinet 2001 ;40(7):539-51.
128. Wylie D C, Voloch M, Lee S, Liu Y H, Cannon-Carlson S, Cutler C, Pramanik B. Carboxyalkylated histidine is a pH-dependent product of pegylation with SC-PEG. Pharm Res 2001; 18(9): 1354-60.
129. Kinstler O B, Brems D N, Lauren S L, Paige A G, Hamburger J B, Treuheit M J. Characterization and stability of N-terminally PEGylated rhG-CSF. Pharm Res 1996;1 3(7):996-1002.
130. Morita T, Horikiri Y, Suzuki T, Yoshino H. Preparation of gelatin microparticles by co-lyophilization with poly(ethylene glycol): characterization and application to entrapment into biodegradable microspheres. Int J Pharm 2001 ;219(1-2):127-37.
131. Lam X M, Duenas E T, Cleland J L. Encapsulation and stabilization of nerve growth factor into poly(lactic-co-glycolic) acid microspheres. J Pharm Sci 2001 ;90(9):1356-65.
132. Kim H K, Park T G. Microencapsulation of dissociable human growth hormone aggregates within poly(D,L-lactic-co-glycolic acid) microparticles for sustained release. Int J Pharm 2001;229(1-2):107-16.
133. Sandor M, Enscore D, Weston P, Mathiowitz E. Effect of protein molecular weight on release from micron-sized PLGA microspheres. J Control Release 2001 ;76(3):297-311.
134. Elvassore N, Bertucco A, Caliceti P. Production of insulin-loaded poly(ethylene glycol)/poly(l-lactide) (PEG/PLA) nanoparticles by gas antisolvent techniques. J Pharm Sci 2001;90(10):1628-36.
135. Scott J K, Loganathan D, Easley R B, Gong X, Goldstein I J. A family of concanavalin A-binding peptides from a hexapeptide epitope library. Proc Natl Acad Sci USA 1992;89(12):5398-402.
136. Valadon P, Nussbaum G, Oh J, Scharff M D. Aspects of antigen mimicry revealed by immunization with a peptide mimetic of Cryptococcus neoformans polysaccharide. J Immunol 1998;161(4):1829-36.
137. Ishikawa D, Taki T. Biocombinatorial Chemistry, a Novel Approach Using Phage-Displayed Libraries in Glycobiology. Trends in Glycoscience and Blycotechnology 1999; 11:277-285.
138. Grothaus M C, Srivastava N, Smithson S L, Kieber-Emmons T, Williams D B, Carlone G M, Westerink M A. Selection of an immunogenic peptide mimic of the capsular polysaccharide of Neisseria meningitidis serogroup A using a peptide display library. Vaccine 2000;18(13):1253-63.
139. Ho M, Springer T. Mac-2, a novel 32,000 Mr mouse macrophage subpopulation-specific antigen defined by monoclonal antibodies. J Immunol 1982;128:1221-1228.
140. Klingbeil C, Hsu D H. Pharmacology and safety assessment of humanized monoclonal antibodies for therapeutic use. Toxicol Pathol 1999;27(1):1-3.
141: Lewis A P, Crowe J S. Generation of humanized monoclonal antibodies by ‘best fit’ framework selection and recombinant polymerase chain reaction. Year Immunol 1993;7:110-8.
142. Merluzzi S, Figini M, Colombatti A, Canevari S, Pucillo C. Humanized antibodies as potential drugs for therapeutic use. Adv Clin Path 2000;4(2):77-85.
143. Leong S R, DeForge L, Presta L, Gonzalez T, Fan A, Reichert M, Chuntharapai A, Kim K J, Tumas D B, Lee W P and others. Adapting pharmacokinetic properties of a humanized anti-interleukin-8 antibody for therapeutic applications using site-specific pegylation. Cytokine 2001;16(3):106-19.
144. Winter G, Harris W J. Humanized antibodies. Trends Pharmacol Sci 1993;14(5):139-43.
145. Hoogenboom H R, Henderikx P, de Haard H. Creating and engineering human antibodies for immunotherapy. Adv Drug Deliv Rev 1998;31(1-2):5-31.
146. Hudson P. Recombinant antibody fragments. Current Opinion in Biotechnol 1998;9:395-402.
147. Campo G M, Avenoso A, Campo S, Ferlazzo A M, Altavilla D, Calatroni A. Efficacy of treatment with glycosaminoglycans on experimental collagen-induced arthritis in rats. Arthritis Res Ther 2003;5(3):R122-31.
148. Paul-Clark M J, Mancini L, Del Soldato P, Flower R J, Perretti M. Potent antiarthritic properties of a glucocorticoid derivative, NCX-1015, in an experimental model of arthritis. Proc Natl Acad Sci USA 2002;99(3):1677-82.

1. A composition comprising an effective amount of N-terminally truncated galectin-3 and a pharmaceutically acceptable carrier.

2. The composition according to claim 1, wherein said N-terminally truncated galectin-3 has a sequence according to SEQ ID NO: 1 and analogues thereof.

3. The composition according to claim 1, wherein said N-terminally truncated galectin-3 is present in an amount sufficient to reduce tumor size.

4. The composition according to claim 1 for use in for treating cancer.

5. The composition according to claim 1, wherein said N-terminally truncated galectin-3 has a sequence as set forth in SEQ ID No:2 and analogues and homologues thereof.

6. The composition according to claim 5, wherein said analogues and homologues include at least one set of polypeptides that include amino acid sequences of SEQ ID No:2 beginning with an amino acid residue from Try-63 through Arg-129 and extends at least to an amino acide residue from Asp-241 through Ile-250.

7. The composition according to claim 1, wherein said composition in formulated for slow release.

8. A method of treating a tumor in a patient by administering to a patient in need of treatment an effective amount of N-terminally truncated galectin-3 according to claim 1, in a pharmaceutically acceptable carrier.

9. The method according to claim 8, wherein said administering step includes administering N-terminally truncated galectin-3 having a sequence as set forth in SEQ ID No:2 and analogues and homologues thereof.

10. The method according to claim 9, wherein said administering step includes administering analogues and homologues that include at least one set of polypeptides that include amino acid sequences of SEQ ID No:2 beginning with an amino acid residue from Try-63 through Arg-129 and extends at least to an amino acide residue from Asp-241 through Ile-250.

11. A treatment treatment for cancer and inflammation comprising an effective amount of N-terminally truncated galectin-3 according to claim 1, and a pharmaceutically acceptable carrier.

12. The treatment according to claim 11, wherein said N-terminally truncated galectin-3 wherein said N-terminally truncated galectin-3 has a sequence as set forth in SEQ ID No:2 and analogues and homologues thereof.

13. The treatment according to claim 12, wherein said analogues and homologues include at least one set of polypeptides that include amino acid sequences of SEQ ID No:2 beginning with an amino acid residue from Try-63 through Arg-129 and extends at least to an amino acide residue from Asp-241 through Ile-250.

14. The treatment according to claim 11, wherein said N-terminally truncated galectin-3 is present in an amount sufficient to prevent metastasis.

15. An anti-cancer treatment comprising an effective amount of a nucleic acid sequence encoding an N-terminally truncated galectin-3 according to claim 1, and a pharmaceutically acceptable carrier.

16. The treatment according to claim 13, wherein said nucleic acid sequence encoding the N-terminally truncated galectin-3 is present in an amount sufficient to prevent metastasis.

17. The treatment according to claim 11, wherein said treatment is formulated for slow release.

18. A nucleic acid sequence encoding for an N-terminally truncated galectin-3.

19. The nucleic acid sequence according to claim 18, wherein said N-terminally truncated galectin-3 wherein said N-terminally truncated galectin-3 has a sequence as set forth in SEQ ID No:2 and analogues and homologues thereof.

20. The nucleic acid sequences according to claim 19, wherein said analogues and homologues include at least one set of polypeptides that include amino acid sequences of SEQ ID No:2 beginning with an amino acid residue from Try-63 through Arg-129 and extends at least to an amino acide residue from Asp-241 through Ile-250.

21. A method of treating a tumor in a patient by administering to a patient in need of treatment an effective amount of a nucleic acid sequence encoding an N-terminally truncated galectin-3 in a pharmaceutically acceptable carrier.

22. The method according to claim 21, wherein said administering step includes administering the N-terminally truncated galectin-3 in a method selected from the group consisting essentially of intramuscularly, orally, intravenously, and locally.

23. An antibody that specifically binds to carbohydrate ligands of galectin-3.

24. The antibody according to claim 23, wherein said antibody is used for treating cancer.
